(19) United States
(12) Patent Application Publication
(10) Pub. No.: US 2024/0167027 A1
(43) Pub. Date: May 23, 2024

(54) SAFE HARBOR LOCI FOR CELL ENGINEERING

(71) Applicant: HANGZHOU QIHAN BIOTECH CO., LTD., Hangzhou, Zhejiang (CN)

(72) Inventors: Luhan YANG, Hangzhou (CN); Yangbin GAO, Hangzhou (CN); Jiabiao HU, Hangzhou (CN); Weihong XU, Hangzhou (CN); Xu FENG, Hangzhou (CN); Meng YANG, Hangzhou (CN)

(73) Assignee: HANGZHOU QIHAN BIOTECH CO., LTD., Hangzhou, Zhejiang (CN)

(21) Appl. No.: 18/555,198

(22) PCT Filed: Apr. 15, 2022
(86) PCT No.: PCT/CN2022/087094

§ 371 Date: Oct. 12, 2023

(30) Foreign Application Priority Data
Apr. 16, 2021 (WO) PCT/CN2021/087819

(51) Int. Cl. C12N 15/11; A61K 35/17; A61K 35/28; C12N 5/0783; C12N 5/0789; C12N 15/63; C12N 15/90

(52) U.S. Cl. CPC C12N 15/11; A61K 35/17; A61K 35/28; C12N 5/0636; C12N 5/0646; C12N 5/0647; C12N 15/63; C12N 15/907; C12N 2510/00; C12N 2800/80

(57) ABSTRACT

Provided herein are safe harbor loci that can be utilized as sites for genetic modification. Safe harbor loci of the disclosure are shown to support sustained transgene expression with minimal silencing, and minimal impact on local or global gene expression. Safe harbor loci disclosed herein can be used in various genetic and cell engineering applications.

### CROSS REFERENCE

This application claims priority to and the benefit of International Patent Application No. PCT/CN2021/087819, which is incorporated herein by reference in its entirety.

### BACKGROUND

Genetic editing technologies have the potential to revolutionize modem medicine, with applications for, e.g., treating cancers, genetic diseases, and a wide spectrum of other diseases. Many genetic editing technologies involve introduction of a desired expression cassette into cells. For example, engineered immune cells can be generated with desirable properties, such as an ability to recognize a specific target via an antigen recognition receptor, and evoke a desired response to the target cell, such as a cytotoxic response to a cancer cell. Diseases that result from genetic mutations can potentially be treated or cured, for example, by introducing and expressing a functional copy of a defective gene. These examples barely scratch the surface of the vast scope of therapeutic possibilities promised by genetic editing technologies. In some cases, a nucleic acid sequence (e.g., transgene) can be introduced into a cell's genome to, e.g., achieve many of the desirable outcomes encompassed by genetic editing.

### SUMMARY

Disclosed herein, in some aspects, is a population of engineered cells, each engineered cell of the population comprising a transgene inserted in a genomic site, wherein upon insertion of the transgene into the genomic site, (i) more than 98.8% of the population maintains expression of the transgene for at least about 15 days, or (ii) more than 97.2% of the population maintains expression of the transgene for at least about 21 days.

Disclosed herein, in some aspects, is a population of engineered cells, each engineered cell of the population comprising a transgene inserted in a genomic site that is not AAVS1, wherein upon insertion of the transgene into the genomic site, (i) more than 68% of the population maintains expression of the transgene for at least about 15 days, or (ii) more than 65% of the population maintains expression of the transgene for at least about 21 days.

Disclosed herein, in some aspects, is a population of engineered cells, each engineered cell of the population comprising a transgene inserted in a genomic site, wherein the engineered cells are pluripotent stem cells, and wherein, upon subjecting the population to differentiation towards a cell lineage, at least about 92% of the differentiating population maintains expression of the transgene.

In some embodiments, the population is subjected to the differentiation for at least about 14 or 21 days. In some embodiments, the cell lineage is selected from the group consisting of embryoid bodies, mesoderm cells, endoderm cells, and ectoderm cells. In some embodiments, the cell lineage comprises hematopoietic stem cells. In some embodiments, the cell lineage comprises NK cells. In some embodiments, the cell lineage comprises T cells.

Disclosed herein, in some aspects is a population of engineered cells, each engineered cell of the population comprising an artificially-induced modification in a genomic site, wherein the artificially-induced modification effects no more than about 10-fold change in expression level of no more than about 100 endogenous genes.

Disclosed herein, in some aspects is a population of engineered cells, each engineered cell of the population comprising an artificially-induced modification in a genomic site, wherein the artificially-induced modification effects no more than about 10-fold change in expression level of no more than about 10 endogenous genes that are within 300 kb of the genomic site.

Disclosed herein, in some aspects is a population of engineered cells, each engineered cell of the population comprising an artificially-induced modification in a genomic site of the engineered cell, wherein the nearest open reading frame to the genomic site in a 5′ or 3′ direction encodes a ribosomal protein, a ubiquitin modulator, an apoptosis regulator, a cell cycle progression regulator, a transcription factor, or a zinc finger-containing protein, wherein the engineered cells are stem cells or NK cells.

Disclosed herein, in some aspects is a population of engineered cells, each engineered cell of the population comprising an artificially-induced modification in a genomic site of the engineered cell, wherein the genomic site is an intergenic region between: (a) FAU and ZNHIT2; (b) RPL3 and SYNGR1; (c) RPLP2 and PIDD1; (d) RPS7 and RNASEH1; (e) THEM4 and S100A10; (f) DDIT4 and ANAPC16; (g) ANXA2 and FOXB1; (h) TOB2 and TEF; (i) NDUFA4 and PHF14; (j) DDX5 and CEP95; (k) PIN4 and RPS4X; (l) PLEKHG2 and RPS16; (m) TRIM41 and RACK1; (n) HINT1 and LYRM7; (o) CFL1 and MUS81; or (p) VPS13B and COX6C.

In some embodiments, the genomic site is adjacent to a promoter operatively coupled to one or more endogenous genes selected from the group consisting of FAU, ZNHIT2, RPL3, RPLP2, RPS7, TMEM4, S100A10, ANAPC16, DDIT4, FOXB1, ANXA2, TEF, TOB2, NDUFA4, DDX5, CEP95, PIN4, RPS4X, PLEKHG2, RPS16, TRIM41, RACK1, HINT1, CFL1, MUS81, VPS13B, and COX6C. In some embodiments, the genomic site has at least 80% sequence identity to one or more sequences from the Genome Reference Consortium Human Build 38 (GRCh38/hg38) human genome selected from the group consisting of: (a) chr11:65,117,969-65,120,057; (b) chr22:39,319,072-39,321,167; (c) chr11:808,403-810,414; (d) chr2:3,574,031-3,576,263; (e) chr1:151,944,637-151,946,598; (f) chr10:72,259,705-72,261,554; (g) chr15:60,126,969-60,128,831; (h) chr22:41,413,106-41,414,808; (i) chr7:10,940,150-10,940,760; (j) chr17:64,506,290-64,506,960; (k) chrX:72,268,950-72,270,750; (l) chr19:39,430,700-39,431,400; (m) chr5:181,235,790-181,236,860; (n) chr5:131,165,330-131,165,510; (o) chr11:65,859,410-65,860,050; and (p) chr8:99,877,580-99,877,850. In some embodiments, after introduction of the engineered cells into a host subject, more than 80% of cells in the population maintain expression of the transgene for at least about two months. In some embodiments, the genomic site is at least 0.5 kb, 1 kb, 2 kb, 3 kb, 4 kb, 5 kb, 6 kb, 7 kb, 8 kb, 9 kb, 10 kb, 11 kb, 12 kb, 13 kb, 14 kb, or 15 kb from the nearest open reading frame in the genome. In some embodiments, the genomic site is at least 1 kb, 2 kb, 3 kb, 4 kb, 5 kb, 6 kb, 7 kb, 8 kb, 9 kb, 10 kb, 15 kb, 20 kb, 25 kb, 30 kb, 35 kb, 40 kb, 50 kb, 60 kb, 70 kb, 75 kb, 80 kb, 90 kb, or 100 kb from the nearest cancer-associated gene in the genome. In some embodiments, the genomic site is at least 1 kb, 2 kb, 3 kb, 4 kb, 5 kb, 6 kb, 7 kb, 8 kb, 9 kb, 10 kb, 15 kb, 20 kb, 25 kb, 30 kb, 35 kb, 40 kb, 50 kb, 60 kb, 70 kb, 75 kb, 80 kb, 90 kb, or 100 kb from the nearest snoRNA-encoding, miRNA-encoding, or lincRNA-encoding gene in the genome. In some embodiments, the artificially-induced modification comprises insertion of a transgene into the genomic site. In some embodiments, the transgene encodes an immune receptor. In some embodiments, the transgene encodes an antigen-recognition receptor. In some embodiments, the transgene encodes an NK receptor. In some embodiments, the transgene encodes a chimeric antigen receptor (CAR). In some embodiments, the chimeric antigen receptor further comprises a costimulatory domain. In some embodiments, the costimulatory domain comprises an amino acid sequence derived from CD27, CD28, 4-1BB, OX40, ICOS, PD-1, LAG-3, 2B4, BTLA, DAP10, DAP12, CTLA-4, or NKG2D, or any combination thereof. In some embodiments, the transgene encodes a cytokine. In some embodiments, the transgene encodes a cytokine receptor. In some embodiments, the engineered cells are stem cells. In some embodiments, the engineered cells are embryonic stem cells. In some embodiments, the engineered cells are induced pluripotent stem cells. In some embodiments, the engineered cells are immune cells. In some embodiments, the engineered cells are NK cells. In some embodiments, the engineered cells are T cells. In some embodiments, the nearest 5′ open reading frame to the genomic site or the nearest 3′ open reading frame to the genomic site encodes a ribosomal protein. In some embodiments, the nearest 5′ open reading frame to the genomic site or the nearest 3′ open reading frame to the genomic site encodes a ubiquitin family member. In some embodiments, the nearest 5′ open reading frame to the genomic site or the nearest 3′ open reading frame to the genomic site encodes a zinc finger-containing protein. In some embodiments, the nearest 5′ open reading frame to the genomic site or the nearest 3′ open reading frame to the genomic site encodes a ubiquitin modulator. In some embodiments, the nearest 5′ open reading frame to the genomic site or the nearest 3′ open reading frame to the genomic site encodes a factor that positively regulates apoptosis. In some embodiments, the nearest 5′ open reading frame to the genomic site or the nearest 3′ open reading frame to the genomic site encodes a factor that negatively regulates apoptosis. In some embodiments, the nearest 5′ open reading frame to the genomic site or the nearest 3′ open reading frame to the genomic site encodes a cell cycle progression regulator. In some embodiments, the nearest 5′ open reading frame to the genomic site or the nearest 3′ open reading frame to the genomic site encodes a transcription factor. In some embodiments, the nearest 5′ open reading frame to the genomic site or the nearest 3′ open reading frame to the genomic site encodes a basic region/leucine zipper (bZIP) transcription factor. In some embodiments, the nearest 5′ open reading frame to the genomic site or the nearest 3′ open reading frame to the genomic site encodes a DNA damage response regulator. In some embodiments, the nearest 5′ open reading frame to the genomic site or the nearest 3′ open reading frame to the genomic site encodes a ubiquitin ligase. In some embodiments, the genomic site is not AAVS1 or H11. In some embodiments, the genomic site is not Rosa26, colA1, TIGRE, or CCR5. In some embodiments, the transgene is operably coupled to a constitutive promoter. In some embodiments, the transgene is operably coupled to an inducible promoter. In some embodiments, the transgene is not operably coupled to an inducible promoter. In some embodiments, the transgene is operably coupled to a tissue-specific promoter. In some embodiments, (i) more than 98.8% of the population maintains constitutive expression of the transgene for at least about 15 days, or (ii) more than 97.2% of the population maintains constitutive expression of the transgene for at least about 21 days. In some embodiments, the genomic site is an intergenic region between TEF and TOB2. In some embodiments, the genomic site is an intergenic region between FAU and ZNHIT2. In some embodiments, the genomic site is an intergenic region between PIDD1 and RPLP2. In some embodiments, the genomic site is an intergenic region between ANAPC16 and DDIT4. In some embodiments, the genomic site is within coordinates chr22:41,413,106-41,414,808 from the Genome Reference Consortium Human Build 38 (GRCh38/hg38) human genome. In some embodiments, the genomic site is within coordinates chr11:65,117,969-65,120,057 from the Genome Reference Consortium Human Build 38 (GRCh38/hg38) human genome. In some embodiments, the genomic site is within coordinates chr11:808,403-810,414 from the Genome Reference Consortium Human Build 38 (GRCh38/hg38) human genome. In some embodiments, the genomic site is within coordinates chr10:72,259,705-72,261,554 from the Genome Reference Consortium Human Build 38 (GRCh38/hg38) human genome.

Disclosed herein, in some aspects, is a vector configured for generation of the engineered cell of any one of the preceding embodiments, the vector comprising a transgene and at least one homology arm, wherein the homology arm is at least 20 nucleotides in length and comprises a nucleotide sequence with at least 90% sequence identity to a corresponding sequence in an intergenic region between: (a) FAU and ZNHIT2; (b) RPL3 and SYNGR1; (c) RPLP2 and PIDD1; (d) RPS7 and RNASEH1; (e) THEM4 and S100A10; (f) DDIT4 and ANAPC16; (g) ANXA2 and FOXB1; (h) TOB2 and TEF; (i) NDUFA4 and PHF14; (j) DDX5 and CEP95; (k) PIN4 and RPS4X; (l) PLEKHG2 and RPS16; (m) TRIM41 and RACK1; (n) HINT1 and LYRM7; (o) CFL1 and MUS81; or (p) VPS13B and COX6C.

In some embodiments, the homology arm is at least 30, at least 40, at least 50, at least 75, at least 100, at least 200, at least 300, at least 400, at least 500, at least 600, at least 700, at least 800, at least 900, or at least 1000 nucleotides in length.

Disclosed herein, in some aspects, is a method of making the engineered cell of any one of the preceding embodiments, the method comprising introducing the transgene or the artificially-induced modification into the genomic site of a cell.

In some embodiments, the introducing the transgene or the artificially-induced modification comprises introducing a double-stranded break in the genomic site. In some embodiments, the double-stranded break is introduced by a nuclease. In some embodiments, the nuclease is a CRISPR-associated (Cas) nuclease, a transcription activator-like effector nuclease (TALEN), or a zinc finger nuclease. In some embodiments, the introducing the transgene or the artificially-induced modification comprises providing a polynucleotide to be integrated into the genomic site by homology-directed repair. In some embodiments, 20 days subsequent to the introducing, (i) a percentage of cells expressing the transgene from a plurality of clones comprising the transgene inserted at the genomic site is higher than (ii) a percentage of cells expressing the transgene from a plurality of clones comprising the transgene inserted at an AAVS1 locus. In some embodiments, (i) an average duration of expression of the transgene from a plurality of clones comprising the transgene inserted at the genomic site is higher than (ii) an average duration of expression of the transgene from a plurality of clones comprising the transgene inserted at an AAVS1 locus. In some embodiments, (i) an average expression level of the transgene from a plurality of clones comprising the transgene inserted at the genomic site is higher than (ii) an average expression level of the transgene from a plurality of clones comprising the transgene inserted at an AAVS1 locus. In some embodiments, expression of the transgene inserted at the genomic site and expression of the transgene inserted at the AAVS1 locus are driven by the same or substantially the same promoter

Disclosed herein, in some aspects, is a pharmaceutical composition comprising the engineered cell or the vector of any one of the preceding embodiments and a pharmaceutically-acceptable excipient, carrier, vehicle, or diluent.

Disclosed herein, in some aspects, is a method of treating a condition in a subject in need thereof, comprising administering to the subject the engineered cell or the pharmaceutical composition of any one of the preceding embodiments.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates stable reporter expression after integration of an expression cassette into safe harbor loci of the disclosure in hESC clones.

FIG. 2 illustrates reporter expression after integration of an expression cassette into control safe harbor loci in hESC clones.

FIG. 3 illustrates stable reporter expression after integration of an expression cassette into a safe harbor locus of the disclosure (hSH8) in hESC clones (each row is for a different clone).

FIG. 4A illustrates reporter expression after integration of an expression cassette into safe harbor loci of the disclosure or AAVS1 in iPSC clones.

FIG. 4B illustrates reporter expression after integration of an expression cassette into AAVS1 in iPSC clones after 9-15 passages.

FIG. 4C illustrates reporter expression after integration of an expression cassette into hSH1 in iPSC clones after 11-21 passages.

FIG. 4D illustrates reporter expression after integration of an expression cassette into hSH8 in iPSC clones after 12-22 passages.

FIG. 5A illustrates stable reporter expression after integration of an expression cassette into a safe harbor locus of the disclosure (hSH8) in iPSC clones that were differentiated into embryoid bodies.

FIG. 5B illustrates loss of reporter expression after integration of an expression cassette into AAVS1 in iPSC clones that were differentiated into embryoid bodies.

FIG. 6 illustrates stable reporter expression after integration of an expression cassette into safe harbor loci of the disclosure (hSH1 and hSH3) in iPSC clones that were differentiated into embryoid bodies.

FIG. 7 illustrates stable reporter expression after integration of an expression cassette into a safe harbor locus of the disclosure (hSH8) in iPSC clones that were differentiated into NK cells, as determined on day 14 of the differentiation protocol.

FIG. 8 illustrates stable reporter expression after integration of an expression cassette into a safe harbor locus of the disclosure (hSH8) in iPSC clones that were differentiated into NK cells, as determined on day 21 of the differentiation protocol.

FIG. 9 illustrates stable reporter expression after integration of an expression cassette into a safe harbor locus of the disclosure (hSH1) in iPSC clones that were differentiated into NK cells, as determined on day 14 of the differentiation protocol.

FIG. 10 illustrates stable reporter expression after integration of an expression cassette into a safe harbor locus of the disclosure (hSH1) in iPSC clones that were differentiated into NK cells, as determined on day 21 of the differentiation protocol.

FIG. 11 illustrates stable reporter expression after integration of an expression cassette into a safe harbor locus of the disclosure (hSH3) in iPSC clones that were differentiated into NK cells, as determined on day 14 of the differentiation protocol.

FIG. 12 illustrates stable reporter expression after integration of an expression cassette into a safe harbor locus of the disclosure (hSH3) in iPSC clones that were differentiated into NK cells, as determined on day 21 of the differentiation protocol.

FIG. 13 illustrates stable in vivo reporter expression after integration of an expression cassette into a safe harbor locus of the disclosure (hSH8) in hESC, implant of the hESC into nude mice, and two months of differentiation into teratomas.

FIG. 14 illustrates stable in vivo reporter expression after integration of an expression cassette into hSH6 and hSH8 safe harbor loci of the disclosure in hESC, implant of the hESC into nude mice, and two months of differentiation into teratomas.

FIG. 15 provides microscopy images of teratoma tissues from mice two months after injection with hESC with expression cassettes at safe harbor loci of the disclosure, demonstrating that the hESC had differentiated into ectoderm, mesoderm, and endoderm lineages.

FIG. 16A provides volcano plots showing differential gene expression in hESC following introduction of transgenes into hSH1, hSH3, hSH6, and hSH8 safe harbor loci of the disclosure.

FIG. 16B provides volcano plots showing differential gene expression in hESC following introduction of transgenes into AAVS1 or H11 loci.

### DETAILED DESCRIPTION

As used in the specification and claims, the singular forms “a,” “an,” and “the” include plural references unless the context clearly dictates otherwise. For example, the term “a chimeric transmembrane receptor” includes a plurality of chimeric transmembrane receptors.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

The term “differentiation” generally refers to a process by which an unspecialized (“uncommitted”) or less specialized cell acquires the features of a specialized cell such as, e.g., an immune cell. A differentiated or differentiation-induced cell is one that has taken on a more specialized (“committed”) position within the lineage of a cell. The term “committed” generally refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

The term “pluripotent” generally refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency can be a continuum of developmental potencies ranging from the incompletely or partially pluripotent cell (e.g., an epiblast stem cell), which is unable to give rise to a complete organism to the more primitive, more pluripotent cell, which is able to give rise to a complete organism (e.g., an embryonic stem cell).

The term “induced pluripotent stem cells” (iPSCs) generally refers to stem cells that are derived from differentiated cells (e.g., differentiated adult, neonatal, or fetal cells) that have been induced or changed (i.e., reprogrammed) into cells capable of differentiating into tissues of all three germ or dermal layers: mesoderm, endoderm, and ectoderm. The iPSCs produced do not refer to cells as they are found in nature. In some cases, iPSCs can be engineered to different directly into committed cells (e.g., natural killer (NK) cells. In some cases, iPSCs can be engineered to differentiate first into tissue-specific stem cells (e.g., hematopoietic stem cells (HSCs)), which can be further induced to differentiate into committed cells (e.g., NK cells).

The term “embryonic stem cell” (ESCs) generally refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. In some cases, ESCs can be engineered to differentiation directly into committed cells (e.g., NK cells). In some cases, ESCs can be engineered to differentiate first into tissue-specific stem cells (e.g., HSCs), which can be further induced to differentiate into committed cells (e.g., NK cells).

The term “isolated stem cells” generally refers to any type of stem cells disclosed herein (e.g., ESCs, HSCs, mesenchymal stem cells (MSCs), etc.) that are isolated, e.g, from a multicellular organism. For example, HSCs can be isolated from a mammal's body, such as a human body. In another example, an embryonic stem cells can be isolated from an embryo.

The term “isolated” generally refers to a cell or a population of cells, which has been separated from its original environment. For example, a new environment of the isolated cells is substantially free of at least one component as found in the environment in which the “un-isolated” reference cells exist. An isolated cell can be a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an isolated cell is partly or completely separated from at least one component, including other substances, cells or cell populations, as it is found in nature or as it is grown, stored or subsisted in non-naturally occurring environments.

The term “hematopoietic stem and progenitor cells,” “hematopoietic stem cells,”, “hematopoietic progenitor cells,” or “hematopoietic precursor cells,” as used interchangeably herein, generally refers to cells which are committed to a hematopoietic lineage but are capable of further hematopoietic differentiation (e.g., into NK cells) and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). In some cases, HSCs can be CD34+ hematopoietic cells capable of giving rise to both mature myeloid and lymphoid cell types including T cells, NK cells and B cells.

The term “immune cell” generally refers to a differentiated hematopoietic cell. Non-limiting examples of an immune cell can include an NK cell, a T cell, a monocyte, an innate lymphocyte, a tumor-infiltrating lymphocyte, a macrophage, a granulocyte, etc.

The term “NK cell” or “Natural Killer cell” generally refers to a subset of peripheral blood lymphocytes defined by the expression of CD56 and/or CD16 and the absence of the T cell receptor (CD3). In some cases, NK cells that are phenotypically CD3− and CD56+, expressing at least one of NKG2C and CD57 (e.g., NKG2C, CD57, or both in same or different degrees), and optionally, CD16, but lack expression of one or more of the following: PLZF, SYK, FceRγ, and EAT-2. In some cases, isolated subpopulations of CD56+NK cells can exhibit expression of CD16, NKG2C, CD57, NKG2D, NCR ligands, NKp30, NKp40, NKp46, activating and inhibitory KIRs, NKG2A and/or DNAM-1.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G] dCTP, [TAMRA] dCTP, [JOE] ddATP, [R6G] ddATP, [FAM] ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or flourescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene, or transgene. A non-native gene or transgene can refer to a gene not normally found in the host organism but which is introduced into the host organism by gene transfer. A non-native gene or transgene can also refer to a gene not in its natural location in the genome of an organism. A non-native gene or transgene can also refer to a naturally occurring nucleic acid or polypeptide sequence that comprises mutations, insertions and/or deletions (e.g., non-native sequence).

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “gene editing moiety” generally refers to a moiety which can edit a nucleic acid sequence, whether exogenous or endogenous to a cell comprising the nucleic acid sequence. In some embodiments, a gene editing moiety regulates expression of a gene by editing a nucleic acid sequence. In some cases, a gene editing moiety can regulate expression of a gene by editing genomic DNA sequence. In some cases, a gene editing moiety can regulate expression of a gene by editing an mRNA template. Editing a nucleic acid sequence can, in some cases, alter the underlying template for gene expression. Alternatively or in addition, a gene editing moiety can be capable of regulating expression or activity of a gene by specifically binding to a target sequence operatively coupled to the gene (or a target sequence within the gene), and regulating the production of mRNA from DNA, such as chromosomal DNA or cDNA. In some cases, a gene editing moiety can recruit or comprise at least one transcription factor that binds to a specific DNA sequence, thereby controlling the rate of transcription of genetic information from DNA to mRNA. A gene editing moiety can itself bind to DNA and regulate transcription by physical obstruction, for example preventing proteins such as RNA polymerase and other associated proteins from assembling on a DNA template. A gene editing moiety can regulate expression of a gene at the translation level, for example, by regulating the production of protein from mRNA template. In some cases, a gene editing moiety can regulate gene expression by affecting the stability of an mRNA transcript.

The term “chimeric polypeptide receptor” generally refers to a non-natural polypeptide receptor comprising one or more antigen binding moieties, each antigen binding moiety capable of binding to a specific antigen. A chimeric polypeptide receptor can be monospecific (i.e., capable of binding to one type of specific antigen). Alternatively, a chimeric polypeptide receptor can be multi-specific (i.e., capable of binding to two or more different types of specific antigens). A chimeric polypeptide receptor can be monovalent (i.e., comprising a single antigen binding moiety). Alternatively, a chimeric polypeptide receptor can be multivalent (i.e., comprising a plurality of antigen binding moieties). In some cases, a chimeric polypeptide receptor can comprise a T-cell receptor (TCR) fusion protein (TFP) or a chimeric antigen receptor (CAR).

The term “antibody” generally refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e. IgA, IgG, IgM, IgD and IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)2, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The term “antigen binding domain” generally refers to a construct exhibiting preferential binding to a specific target antigen. An antigen binding domain can be a polypeptide construct, such as an antibody, modification thereof, fragment thereof, or a combination thereof. The antigen binding domain can be any antibody as disclosed herein, or a functional variant thereof. Non-limiting examples of an antigen binding domain can include a murine antibody, a human antibody, a humanized antibody, a camel Ig, a shark heavy-chain-only antibody (VNAR), Ig NAR, a chimeric antibody, a recombinant antibody, or antibody fragment thereof. Non-limiting examples of antibody fragment include Fab, Fab′, F(ab)′2, F(ab)′3, Fv, single chain antigen binding fragment (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody. “antigen binding domain” can also refer to non-antibody molecules that specifically bind to a target, for example DARPins, ligands that bind to receptors, receptors that bind to ligands, etc.

The term “enhanced expression,” “increased expression,” or “upregulated expression” generally refers to production of a moiety of interest (e.g., a polynucleotide or a polypeptide) to a level that is above a normal level of expression of the moiety of interest in a host strain (e.g., a host cell). The normal level of expression can be substantially zero (or null) or higher than zero. The moiety of interest can comprise an endogenous gene or polypeptide construct of the host strain. The moiety of interest can comprise a heterologous gene or polypeptide construct that is introduced to or into the host strain. For example, a heterologous gene encoding a polypeptide of interest can be knocked-in (KI) to a genome of the host strain for enhanced expression of the polypeptide of interest in the host strain.

The term “enhanced activity,” “increased activity,” or “upregulated activity” generally refers to activity of a moiety of interest (e.g., a polynucleotide or a polypeptide) that is modified to a level that is above a normal level of activity of the moiety of interest in a host strain (e.g., a host cell). The normal level of activity can be substantially zero (or null) or higher than zero. The moiety of interest can comprise a polypeptide construct of the host strain. The moiety of interest can comprise a heterologous polypeptide construct that is introduced to or into the host strain. For example, a heterologous gene encoding a polypeptide of interest can be knocked-in (KI) to a genome of the host strain for enhanced activity of the polypeptide of interest in the host strain.

The term “reduced expression,” “decreased expression,” or “downregulated expression” generally refers to a production of a moiety of interest (e.g., a polynucleotide or a polypeptide) to a level that is below a normal level of expression of the moiety of interest in a host strain (e.g., a host cell). The normal level of expression is higher than zero. The moiety of interest can comprise an endogenous gene or polypeptide construct of the host strain. In some cases, the moiety of interest can be knocked-out or knocked-down in the host strain. In some examples, reduced expression of the moiety of interest can include a complete inhibition of such expression in the host strain.

The term “reduced activity,” “decreased activity,” or “downregulated activity” generally refers to activity of a moiety of interest (e.g., a polynucleotide or a polypeptide) that is modified to a level that is below a normal level of activity of the moiety of interest in a host strain (e.g., a host cell). The normal level of activity is higher than zero. The moiety of interest can comprise an endogenous gene or polypeptide construct of the host strain. In some cases, the moiety of interest can be knocked-out or knocked-down in the host strain. In some examples, reduced activity of the moiety of interest can include a complete inhibition of such activity in the host strain.

The term “subject,” “individual,” or “patient,” as used interchangeably herein, generally refers to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The term “treatment” or “treating” generally refers to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” generally refers to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” generally refers to that quantity of a composition that is sufficient to delay the manifestation, arrest the progression, relieve or alleviate at least one symptom of a disorder treated by the methods of the present disclosure.

### I. OVERVIEW

Genetic editing technologies have the potential to revolutionize modem medicine, with applications for, e.g., treating cancers, genetic diseases, and a wide spectrum of other diseases.

In some cases, a nucleic acid sequence (e.g., an expression cassette comprising a transgene) can be introduced into a cell's genome. In doing so, it can be important to select a site in the genome that may not significantly disrupt expression of other genes that may be important to, for example, suppressing neoplastic transformation, or other important cellular functions. In some cases, it can be important to select a site that may allow for sustained expression of the transgene; its presence in the genome is of little value if silencing suppresses transgene expression. However, there is a lack of known sites that fit these criteria.

### II. SAFE HARBOR LOCI AND ENGINEERED CELLS

Provided herein are safe harbor loci that can be utilized as sites for genetic modification. Safe harbor loci of the disclosure can support sustained transgene expression with minimal silencing, and/or minimal impact on local or global gene expression. Safe harbor loci disclosed herein can be used in various genetic and cell engineering applications. Insert sequences, such as expression cassettes comprising transgenes, can be introduced into safe harbor loci disclosed herein in any desirable cell type. Transgenes can be introduced into stem cells, which can then be differentiated into a lineage or specific cell type of interest, for example, to generate engineered immune cells, such as engineered NK cells. Transgenes can be introduced into immune cells, for example, T cells or NK cells. Any desirable expression cassette(s) and transgene(s) can be introduced into the safe harbor loci, including for example, immune receptors, cytokines, cytokine receptors, chimeric fusion proteins, transcription factors, or any other transgene with useful applications. The transgenes can be operatively coupled to a range of regulatory elements, for example, promoters, such as inducible promoters, constitutive promoters, or tissue-specific promoters. Certain characteristics of the genetic context of the safe harbor loci are also disclosed herein, such as adjacent genes and classes thereof in the 5′ and/or 3′ direction, and distances from open reading frames, cancer-associated genes, snoRNA-encoding, miRNA-encoding, and lincRNA-encoding genes.

Vectors for introducing modifications into the safe harbor loci, populations of engineered cells comprising the modifications, methods of making the cells, compositions comprising the cells and/or vectors, and methods of using the cells for therapeutic applications are also disclosed.

In some aspects, the present disclosure provides a population of engineered cells, each engineered cell of the population comprising a transgene inserted in a genomic site. Upon insertion of the transgene into the genomic site, more than 90% (e.g., 98.8%) of the population can maintain expression of the transgene for at least about 15 days. Alternatively or additionally, upon insertion of the transgene into the genomic site, more than 90% (e.g., 97.2%) of the population can maintain expression of the transgene for at least about 21 days.

In some embodiments, more than 95%, more than 95.1%, more than 95.2%, more than 95.3%, more than 95.4%, more than 95.5%, more than 95.6%, more than 95.7%, more than 95.8%, more than 95.9%, more than 96%, more than 96.1%, more than 96.2%, more than 96.3%, more than 96.4%, more than 96.5%, more than 96.6%, more than 96.7%, more than 96.8%, more than 96.9%, more than 97%, more than more than 97.1%, more than 97.2%, more than 97.3%, more than 97.4%, more than 97.5%, more than 97.6%, more than 97.7%, more than 97.8%, more than 97.9%, more than 98%, more than 98.1%, more than 98.2%, more than 98.3%, more than 98.4%, more than 98.5%, more than 98.6%, more than 98.7%, more than 98.8%, more than 98.9%, more than 99%, more than 99.1%, more than 99.2%, more than 99.3%, more than 99.4%, more than 99.5%, more than 99.6%, more than 99.7%, more than 99.8%, more than 99.85%, more than 99.9%, more than 99.95%, or more than 99.99% of the population can maintain expression of the transgene for at least about 15 days (e.g., at least about 15, 16, 17, 18, 19, 20, or more days). In some embodiments, more than 95%, more than 95.1%, more than 95.2%, more than 95.3%, more than 95.4%, more than 95.5%, more than 95.6%, more than 95.7%, more than 95.8%, more than 95.9%, more than 96%, more than 96.1%, more than 96.2%, more than 96.3%, more than 96.4%, more than 96.5%, more than 96.6%, more than 96.7%, more than 96.8%, more than 96.9%, more than 97%, more than more than 97.1%, more than 97.2%, more than 97.3%, more than 97.4%, more than 97.5%, more than 97.6%, more than 97.7%, more than 97.8%, more than 97.9%, more than 98%, more than 98.1%, more than 98.2%, more than 98.3%, more than 98.4%, more than 98.5%, more than 98.6%, more than 98.7%, more than 98.8%, more than 98.9%, more than 99%, more than 99.1%, more than 99.2%, more than 99.3%, more than 99.4%, more than 99.5%, more than 99.6%, more than 99.7%, more than 99.8%, more than 99.85%, more than 99.9%, more than 99.95%, or more than 99.99% of the population can maintain expression of the transgene for at least about 21 days (e.g., at least about 21, 22, 23, 24, 25, 26, 27, 28, 29, 30, or more days).

In some aspects, the present disclosure provides a population of engineered cells each engineered cell of the population comprising a transgene inserted in a genomic site that is not an adeno-associated virus integration site (AAVS), for example, that is not AAVS1. More than 50% (e.g., 68%) of the population can maintain expression of the transgene for at least about 15 days. Alternatively or additionally, more than 50% (e.g., 65%) of the population can maintain expression of the transgene for at least about 21 days.

In some embodiments, more than 50%, more than 55%, more than 60%, more than 61%, more than 62%, more than 63%, more than 64%, more than 65%, more than 66%, more than 67%, more than 68%, more than 69%, more than 70%, more than 71%, more than 72%, more than 73%, more than 74%, more than 75%, more than 76%, more than 77%, more than 78%, more than 79%, more than 80%, more than 81%, more than 82%, more than 83%, more than 84%, more than 85%, more than 86%, more than 87%, more than 88%, more than 89%, more than 90%, more than 91%, more than 92%, more than 93%, more than 94%, more than 95%, more than 95.5%, more than 96%, more than 96.5%, more than 97%, more than 97.5%, more than 98%, more than 98.5%, more than 99%, or more than 99.5% of the population maintain expression of the transgene from the genomic site (e.g., non-AAVS1 genomic site) for at least about 15 days (e.g., at least about 15, 16, 17, 18, 19, 20, or more days). In some embodiments, more than 20%, more than 25%, more than 30%, more than 35%, more than 40%, more than 45%, more than 50%, more than 55%, more than 60%, more than 61%, more than 62%, more than 63%, more than 64%, more than 65%, more than 66%, more than 67%, more than 68%, more than 69%, more than 70%, more than 71%, more than 72%, more than 73%, more than 74%, more than 75%, more than 76%, more than 77%, more than 78%, more than 79%, more than 80%, more than 81%, more than 82%, more than 83%, more than 84%, more than 85%, more than 86%, more than 87%, more than 88%, more than 89%, more than 90%, more than 91%, more than 92%, more than 93%, more than 94%, more than 95%, more than 95.5%, more than 96%, more than 96.5%, more than 97%, more than 97.5%, more than 98%, more than 98.5%, more than 99%, or more than 99.5% of the population maintain expression of the transgene from the genomic site (e.g., non-AAVS1 genomic site) for at least about 21 days (e.g., at least about 21, 22, 23, 24, 25, 26, 27, 28, 29, 30, or more days).

In some aspects, the present disclosure provides a population of engineered cells each engineered cell of the population comprising a transgene inserted in a genomic site. The engineered cells can be stem cells (e.g., pluripotent stem cells). Upon subjecting the population to differentiation towards a cell lineage, or after differentiation into a particular cell lineage or specific cell type, at least about 10% (e.g., 80%) of the cells in the population can maintain expression of the transgene.

In some embodiments, upon subjecting the population to differentiation towards a cell lineage, or after differentiation into a particular cell lineage or specific cell type, at least about 10%, at least about 15%, at least about 20%, at least about 25%, at least about 30%, at least about 35%, at least about 40%, at least about 45%, at least about 50%, at least about 55%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or at least about 99.9% of the cells in the population can maintain expression of the transgene. In some embodiments, the cell lineage comprises embryoid bodies, mesoderm cells, endoderm cells, and ectoderm cells, hematopoietic stem cells, hematopoietic cells, immune cells, myeloid cells, lymphoid cells, lymphocytes, T cells, CD4+ T cells, CD8+ T cells, alpha-beta T cells, gamma-delta T cells, T regulatory cells (Tregs), cytotoxic T lymphocytes, Th1 cells, Th2 cells, Th17 cells, Th9 cells, naïve T cells, memory T cells, effector T cells, effector-memory T cells (TEM), central memory T cells (TCM), resident memory T cells (TRM), follicular helper T cells (TFH), naïve T cells, Natural killer T cells (NKTs), tumor-infiltrating lymphocytes (TILs), Natural killer cells (NKs), Innate Lymphoid Cells (ILCs), ILC1 cells, ILC2 cells, ILC3 cells, lymphoid tissue inducer (LTi) cells, B cells, B1 cells, B1a cells, B1b cells, B2 cells, plasma cells, B regulatory cells, memory B cells, marginal zone B cells, follicular B cells, germinal center B cells, antigen presenting cells (APCs), monocytes, macrophages, M1 macrophages, M2 macrophages, tissue-associated macrophages, dendritic cells, plasmacytoid dendritic cells, neutrophils, mast cells, basophils, eosinophils, or any combination thereof.

In some embodiments, the population is subjected to the differentiation for at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 25, at least about 30, at least about 35, at least about 40, at least about 45, at least about 50, at least about 60, or at least about 70 days. In some embodiments, the population is subjected to the differentiation for at least about 14 days. In some embodiments, the population is subjected to the differentiation for at least about 21 days.

In some aspects, the present disclosure provides a population of engineered cells each engineered cell of the population comprising an artificially-induced modification in a genomic site. In some cases the artificially-induced modification effects no more than about a 500-fold change (e.g., no more than about a 10-fold change) in expression level of no more than about 1000 (e.g., no more than about 100) endogenous genes.

In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, or no more than about 500 fold change in expression of no more than about 5, 6, 7, 8, 9, 10, 15, 20, 25, 30, 40, 50, 55, 60, 70, 80, 90, 100, 150, 200, 250, 300, 350, 400, 450, 500, 550, 600, 650, 700, 750, 800, 850, 900, 950, or 1000 endogenous genes. In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, or no more than about 500 fold change in expression of no more than about 100 endogenous genes. In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, or no more than about 500 fold change in expression of no more than about 55 endogenous genes.

In some embodiments, the artificially-induced modification effects no more than about 2-fold change in expression of no more than about 5, no more than about 6, no more than about 7, no more than about 8, no more than about 9, no more than about 10, no more than about 15, no more than about 20, no more than about 25, no more than about 30, no more than about 40, no more than about 50, no more than about 55, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, no more than about 550, no more than about 600, no more than about 650, no more than about 700, no more than about 750, no more than about 800, no more than about 850, no more than about 900, no more than about 950, or no more than about 1000 endogenous genes. In some embodiments, the artificially-induced modification effects no more than about 2-fold change in expression of no more than 50 endogenous genes. In some embodiments, the artificially-induced modification effects no more than about 2-fold change in expression of no more than 55 endogenous genes. In some embodiments, the artificially-induced modification effects no more than about 2-fold change in expression of no more than 60 endogenous genes. In some embodiments, the artificially-induced modification effects no more than about 2-fold change in expression of no more than 70 endogenous genes. In some embodiments, the artificially-induced modification effects no more than about 2-fold change in expression of no more than 80 endogenous genes. In some embodiments, the artificially-induced modification effects no more than about 2-fold change in expression of no more than 100 endogenous genes.

In some aspects, the present disclosure provides a population of engineered cells each engineered cell of the population comprising an artificially-induced modification in a genomic site. In some cases the artificially-induced modification effects no more than about a 500-fold change (e.g., no more than about a 10-fold change) in expression level of no more than about 1000 endogenous genes (e.g., no more than about 100 endogenous genes) that are within at most about 1000 kb (e.g., at most about 300 kb) of the genomic site.

In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, or no more than about 500 fold change in expression of no more than about 1, 2, 3, 4, 5, 6, 7, 8, 9, 10, 15, 20, 25, 30, 40, 50, 60, 70, 80, 90, 100, 150, 200, 250, 300, 350, 400, 450, 500, 550, 600, 650, 700, 750, 800, 850, 900, 950, or 1000 endogenous genes that are at most about 500 kb, 400 kb, 300 kb, 200 kb, 100 kb, 50 kb, 40 kb, 30 kb, 25 kb, 20 kb, 15 kb, 10 kb, or 5 kb from the genomic site. In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, or no more than about 500 fold change in expression of no more than about 5 endogenous genes that are at most about 300 kb from the genomic site. In some embodiments, the artificially-induced modification effects no more than about 2-fold change in expression of no more than about 5 endogenous genes that are at most about 300 kb from the genomic site. In some embodiments, the artificially-induced modification effects no more than about 2-fold change in expression of no more than about 3 endogenous genes that are at most about 300 kb from the genomic site. In some embodiments, the artificially-induced modification effects no more than about 2-fold change in expression of no more than about 2 endogenous genes that are at most about 300 kb from the genomic site. In some embodiments, the artificially-induced modification effects no more than about 2-fold change in expression of no more than about 1 endogenous genes that are at most about 300 kb from the genomic site. In some embodiments, the artificially-induced modification effects no more than about 2-fold change in expression of any endogenous genes that are at most about 300 kb from the genomic site.

In some aspects, the present disclosure provides a population of engineered cells, each engineered cell of the population comprising an artificially-induced modification in a genomic site. The nearest open reading frame to the genomic site in a 5′ or 3′ direction can encode a ribosomal protein, a ubiquitin modulator, an apoptosis regulator, a cell cycle progression regulator, a transcription factor, or a zinc finger-containing protein. The engineered cell can be a stem cell or an NK cell.

In some aspects, the present disclosure provides a population of engineered cells, each engineered cell of the population comprising an artificially-induced modification in a genomic site. The genomic site can be an intergenic region between: (a) FAU and ZNHIT2; (b) RPL3 and SYNGR1; (c) RPLP2 and PIDD1; (d) RPS7 and RNASEH1; (e) THEM4 and S100A10; (f) DDIT4 and ANAPC16; (g) ANXA2 and FOXB1; (h) TOB2 and TEF; (i) NDUFA4 and PHF14; (j) DDX5 and CEP95; (k) PIN4 and RPS4X; (l) PLEKHG2 and RPS16; (m) TRIM41 and RACK1; (n) HINT1 and LYRM7; (o) CFL1 and MUS81; or (p) VPS13B and COX6C. The genomic site can be an intergenic region selected from the group consisting of: (a) FAU and ZNHIT2; (b) RPL3 and SYNGR1; (c) RPLP2 and PIDD1; (d) RPS7 and RNASEH1; (e) THEM4 and S100A10; (f) DDIT4 and ANAPC16; (g) ANXA2 and FOXB1; (h) TOB2 and TEF; (i) NDUFA4 and PHF14; (j) DDX5 and CEP95; (k) PIN4 and RPS4X; (l) PLEKHG2 and RPS16; (m) TRIM41 and RACK1; (n) HINT1 and LYRM7; (o) CFL1 and MUS81; and (p) VPS13B and COX6C.

In some embodiments of any one of the populations of engineered cells disclosed herein, the genomic site is adjacent to a promoter that is operatively coupled to one or more endogenous genes comprising FAU, ZNHIT2, RPL3, RPLP2, RPS7, TMEM4, S100A10, ANAPC16, DDIT4, FOXB1, ANXA2, TEF, TOB2, NDUFA4, DDX5, CEP95, PIN4, RPS4X, PLEKHG2, RPS16, TRIM41, RACK1, HINT1, CFL1, MUS81, VPS13B, or COX6C. The genomic site can be adjacent to a promoter that is operatively coupled to one or more endogenous genes selected from the group consisting of FAU, ZNHIT2, RPL3, RPLP2, RPS7, TMEM4, S100A10, ANAPC16, DDIT4, FOXB1, ANXA2, TEF, TOB2, NDUFA4, DDX5, CEP95, PIN4, RPS4X, PLEKHG2, RPS16, TRIM41, RACK1, HINT1, CFL1, MUS81, VPS13B, and COX6C.

In some embodiments of any one of the populations of engineered cells disclosed herein, the genomic site has at least 80% sequence identity to one or more sequences from the human genome comprising (a) chr11:65,117,969-65,120,057; (b) chr22:39,319,072-39,321,167; (c) chr11:808,403-810,414; (d) chr2:3,574,031-3,576,263; (e) chr1:151,944,637-151,946,598; (f) chr10:72,259,705-72,261,554; (g) chr15:60,126,969-60,128,831; (h) chr22:41,413,106-41,414,808; (i) chr7:10,940,150-10,940,760; (j) chr17:64,506,290-64,506,960; (k) chrX:72,268,950-72,270,750; (l) chr19:39,430,700-39,431,400; (m) chr5:181,235,790-181,236,860; (n) chr5:131,165,330-131,165,510; (o) chr11:65,859,410-65,860,050; or (p) chr8:99,877,580-99,877,850. The genomic site can have at least 80% sequence identity to one or more sequences from the human genome selected from the group consisting of: (a) chr11:65,117,969-65,120,057; (b) chr22:39,319,072-39,321,167; (c) chr11:808,403-810,414; (d) chr2:3,574,031-3,576,263; (e) chr1:151,944,637-151,946,598; (f) chr10:72,259,705-72,261,554; (g) chr15:60,126,969-60,128,831; (h) chr22:41,413,106-41,414,808; (i) chr7:10,940,150-10,940,760; (j) chr17:64,506,290-64,506,960; (k) chrX:72,268,950-72,270,750; (l) chr19:39,430,700-39,431,400; (m) chr5:181,235,790-181,236,860; (n) chr5:131,165,330-131,165,510; (o) chr11:65,859,410-65,860,050; and (p) chr8:99,877,580-99,877,850 of, for example, Genome Reference Consortium Human Build 38 (GRCh38/hg38).

In some embodiments of any one of the populations of engineered cells disclosed herein, after introduction of the engineered cells into a host subject, more than 1%, more than 2%, more than 3%, more than 4%, more than 5%, more than 6%, more than 7%, more than 8%, more than 9%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%, more than 95.5%, more than 96%, more than 96.5%, more than 97%, more than 97.5%, more than 98%, more than 98.5%, more than 99%, more than 99.1%, more than 99.2%, more than 99.3%, more than 99.4%, more than 99.5%, more than 99.6%, more than 99.7%, more than 99.8%, more than 99.9%, or more than 99.95% of cells in the population maintain expression of the transgene for at least 1, at least about 2, at least about 3, at least about 4, at least about 5, at least about 6, at least about 7, at least about 8, at least about 9, at least about 10, at least about 11, or at least about 12 months.

In some embodiments of any one of the populations of engineered cells disclosed herein, after introduction of the engineered cells into a host subject, more than 1%, more than 2%, more than 3%, more than 4%, more than 5%, more than 6%, more than 7%, more than 8%, more than 9%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%, more than 95.5%, more than 96%, more than 96.5%, more than 97%, more than 97.5%, more than 98%, more than 98.5%, more than 99%, more than 99.1%, more than 99.2%, more than 99.3%, more than 99.4%, more than 99.5%, more than 99.6%, more than 99.7%, more than 99.8%, more than 99.9%, or more than 99.95% of cells in the population maintain expression of the transgene for at least about two months.

In some embodiments of any one of the populations of engineered cells disclosed herein, after introduction of the engineered cells into a host subject, more than 80% of cells in the population maintain expression of the transgene for at least about two months.

In some embodiments of any one of the populations of engineered cells disclosed herein, the artificially-induced modification comprises insertion of a transgene and/or expression cassette into the genomic site.

In some embodiments of any one of the populations of engineered cells disclosed herein, the transgene encodes an immune receptor. In some embodiments of any one of the populations of engineered cells disclosed herein, the transgene encodes antigen-recognition receptor. In some embodiments of any one of the populations of engineered cells disclosed herein, the transgene encodes an NK receptor. In some embodiments of any one of the populations of engineered cells disclosed herein, the transgene encodes a chimeric antigen receptor (CAR). In some embodiments of any one of the populations of engineered cells disclosed herein, the transgene encodes a cytokine receptor. In some embodiments of any one of the populations of engineered cells disclosed herein, the transgene encodes a cytokine.

In some embodiments of any one of the populations of engineered cells disclosed herein, the transgene is operably coupled to a constitutive promoter. In some embodiments of any one of the populations of engineered cells disclosed herein, the transgene is operably coupled to an inducible promoter. In some embodiments of any one of the populations of engineered cells disclosed herein, the transgene is operably coupled to a tissue-specific promoter.

In some embodiments of any one of the populations of engineered cells disclosed herein, the transgene is not operably coupled to a constitutive promoter. In some embodiments of any one of the populations of engineered cells disclosed herein, the transgene is not operably coupled to an inducible promoter. In some embodiments of any one of the populations of engineered cells disclosed herein, the transgene is not operably coupled to a tissue-specific promoter.

In some embodiments of any one of the populations of engineered cells disclosed herein, the artificially-induced modification is at least 0.5 kb, at least 1 kb, at least 2 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, or at least 15 kb from the nearest open reading frame in the genome.

In some embodiments of any one of the populations of engineered cells disclosed herein, the artificially-induced modification is at least 0.5 kb, at least 1 kb, at least 2 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, at least 15 kb, at least 20 kb, at least 25 kb, at least 30 kb, at least 35 kb, at least 40 kb, at least 50 kb, at least 60 kb, at least 70 kb, at least 75 kb, at least 80 kb, at least 90 kb, at least 100 kb, at least 110 kb, at least 120 kb, at least 130 kb, at least 140 kb, at least 150 kb, at least 160 kb, at least 170 kb, at least 180 kb, at least 190 kb, at least 200 kb, at least 210 kb, at least 220 kb, at least 230 kb, at least 240 kb, at least 250 kb, at least 260 kb, at least 270 kb, at least 280 kb, at least 290 kb, or at least 300 kb from the nearest cancer-associated gene in the genome. A cancer-associated gene can be, for example, a gene listed in Sondka et al. The COSMIC Cancer Gene Census: describing genetic dysfunction across all human cancers. Nature Reviews Cancer, 2018, 18(11): 696-705; or Martínez-Jiménez et al. A compendium of mutational cancer driver genes. Nature Reviews Cancer, 2020: 1-18, each of which is incorporated herein by reference in its entirety.

In some embodiments, a cancer associated gene is or comprises A1CF, ABI1, ABL1, ABL2, ACKR3, ACSL3, ACSL6, ACVR1, ACVR2A, AFDN, AFF1, AFF3, AFF4, AKAP9, AKT1, AKT2, AKT3, ALDH2, ALK, AMER1, ANK1, APC, APOBEC3B, AR, ARAF, ARHGAP26, ARHGAP5, ARHGEF10, ARHGEF10L, ARHGEF12, ARID1A, ARID1B, ARID2, ARNT, ASPSCR1, ASXL1, ASXL2, ATF1, ATIC, ATM, ATP1A1, ATP2B3, ATR, ATRX, AXIN1, AXIN2, B2M, BAP1, BARD1, BAX, BAZ1A, BCL10, BCL11A, BCL11B, BCL2, BCL2L12, BCL3, BCL6, BCL7A, BCL9, BCL9L, BCLAF1, BCOR, BCORL1, BCR, BIRC3, BIRC6, BLM, BMP5, BMPR1A, BRAF, BRCA1, BRCA2, BRD3, BRD4, BRIP1, BTG1, BTK, BUB1B, C15orf65, CACNAlD, CALR, CAMTA1, CANT1, CARD11, CARS, CASP3, CASP8, CASP9, CBFA2T3, CBFB, CBL, CBLB, CBLC, CCDC6, CCNB1IP1, CCNC, CCND1, CCND2, CCND3, CCNE1, CCR4, CCR7, CD209, CD274, CD28, CD74, CD79A, CD79B, CDC73, CDH1, CDH10, CDH11, CDH17, CDK12, CDK4, CDK6, CDKN1A, CDKN1B, CDKN2A, CDKN2C, CDX2, CEBPA, CEP89, CHCHD7, CHD2, CHD4, CHEK2, CHIC2, CHST11, CIC, CIITA, CLIP1, CLP1, CLTC, CLTCL1, CNBD1, CNBP, CNOT3, CNTNAP2, CNTRL, COL1A1, COL2A1, COL3A1, COX6C, CPEB3, CREB1, CREB3L1, CREB3L2, CREBBP, CRLF2, CRNKL1, CRTC1, CRTC3, CSF1R, CSF3R, CSMD3, CTCF, CTNNA2, CTNNB1, CTNND1, CTNND2, CUL3, CUX1, CXCR4, CYLD, CYP2C8, CYSLTR2, DAXX, DCAF12L2, DCC, DCTN1, DDB2, DDIT3, DDR2, DDX10, DDX3X, DDX5, DDX6, DEK, DGCR8, DICERi, DNAJB1, DNM2, DNMT3A, DROSHA, DUX4L1, EBF1, ECT2L, EED, EGFR, EIF1AX, EIF3E, EIF4A2, ELF3, ELF4, ELK4, ELL, ELN, EML4, EP300, EPAS1, EPHA3, EPHA7, EPS15, ERBB2, ERBB3, ERBB4, ERC1, ERCC2, ERCC3, ERCC4, ERCC5, ERG, ESR1, ETNK1, ETV1, ETV4, ETV5, ETV6, EWSR1, EXT1, EXT2, EZH2, EZR, FAM131B, FAM135B, FAM47C, FANCA, FANCC, FANCD2, FANCE, FANCF, FANCG, FAS, FAT1, FAT3, FAT4, FBLN2, FBXO11, FBXW7, FCGR2B, FCRL4, FEN1, FES, FEV, FGFR1, FGFR1OP, FGFR2, FGFR3, FGFR4, FH, FHIT, FIP1L1, FKBP9, FLCN, FLIl, FLNA, FLT3, FLT4, FNBP1, FOXA1, FOXL2, FOXO1, FOXO3, FOXO4, FOXP1, FOXR1, FSTL3, FUBP1, FUS, GAS7, GATA1, GATA2, GATA3, GLI1, GMPS, GNA11, GNAQ, GNAS, GOLGA5, GOPC, GPC3, GPC5, GPHN, GRIN2A, GRM3, H3F3A, H3F3B, HERPUD1, HEY1, HIF1A, HIP1, HIST1H3B, HIST1H4I, HLA-A, HLF, HMGA1, HMGA2, HMGN2P46, HNF1A, HNRNPA2B1, HOOK3, HOXA11, HOXA13, HOXA9, HOXC11, HOXC13, HOXD11, HOXD13, HRAS, HSP90AA1, HSP90AB1, ID3, IDH1, IDH2, IGF2BP2, IGH, IGK, IGL, IKBKB, IKZF1, IL2, IL21R, IL6ST, IL7R, IRF4, IRS4, ISX, ITGAV, ITK, JAK1, JAK2, JAK3, JAZF1, JUN, KAT6A, KAT6B, KAT7, KCNJ5, KDM5A, KDM5C, KDM6A, KDR, KDSR, KEAP1, KIAA1549, KIF5B, KIT, KLF4, KLF6, KLK2, KMT2A, KMT2C, KMT2D, KNL1, KNSTRN, KRAS, KTN1, LARP4B, LASP1, LATS1, LATS2, LCK, LCP1, LEF1, LEPROTL1, LHFPL6, LIFR, LMNA, LMO1, LMO2, LPP, LRIG3, LRP1B, LSM14A, LYL1, LZTR1, MACC1, MAF, MAFB, MALAT1, MALT1, MAML2, MAP2K1, MAP2K2, MAP2K4, MAP3K1, MAP3K13, MAPK1, MAX, MB21D2, MDM2, MDM4, MDS2, MECOM, MED12, MEN1, MET, MGMT, MITF, MLF1, MLH1, MLLT1, MLLT10, MLLT11, MLLT3, MLLT6, MN1, MNX1, MPL, MRTFA, MSH2, MSH6, MSI2, MSN, MTCP1, MTOR, MUC1, MUC16, MUC4, MUTYH, MYB, MYC, MYCL, MYCN, MYD88, MYH11, MYH9, MYO5A, MYOD1, N4BP2, NAB2, NACA, NBEA, NBN, NCKIPSD, NCOA1, NCOA2, NCOA4, NCOR1, NCOR2, NDRG1, NF1, NF2, NFATC2, NFE2L2, NFIB, NFKB2, NFKBIE, NIN, NKX2-1, NONO, NOTCHI, NOTCH2, NPM1, NR4A3, NRAS, NRG1, NSD1, NSD2, NSD3, NT5C2, NTHL1, NTRK1, NTRK3, NUMA1, NUP214, NUP98, NUTM1, NUTM2B, NUTM2D, OLIG2, OMD, P2RY8, PABPC1, PAFAH1B2, PALB2, PATZ1, PAX3, PAX5, PAX7, PAX8, PBRM1, PBX1, PCBP1, PCM1, PDCD1LG2, PDE4DIP, PDGFB, PDGFRA, PDGFRB, PER1, PHF6, PHOX2B, PICALM, PIK3CA, PIK3CB, PIK3R1, PIM1, PLAG1, PLCG1, PML, PMS1, PMS2, POLD1, POLE, POLG, POLQ, POT1, POU2AF1, POU5F1, PPARG, PPFIBP1, PPM1D, PPP2R1A, PPP6C, PRCC, PRDM1, PRDM16, PRDM2, PREX2, PRF1, PRKACA, PRKAR1A, PRKCB, PRPF40B, PRRX1, PSIP1, PTCH1, PTEN, PTK6, PTPN11, PTPN13, PTPN6, PTPRB, PTPRC, PTPRD, PTPRK, PTPRT, PWWP2A, QKI, RABEP1, RAC1, RAD17, RAD21, RAD51B, RAF1, RALGDS, RANBP2, RAP1GDS1, RARA, RB1, RBM10, RBM15, RECQL4, REL, RET, RFWD3, RGPD3, RGS7, RHOA, RHOH, RMI2, RNF213, RNF43, ROBO2, ROS1, RPL10, RPL22, RPL5, RPN1, RSPO2, RSPO3, RUNX1, RUNX1T1, S100A7, SALL4, SBDS, SDC4, SDHA, SDHAF2, SDHB, SDHC, SDHD, 44444, 44445, 44448, SET, SETBP1, SETD1B, SETD2, SETDB1, SF3B1, SFPQ, SFRP4, SGK1, SH2B3, SH3GL1, SHTN1, SIRPA, SIX1, SIX2, SKI, SLC34A2, SLC45A3, SMAD2, SMAD3, SMAD4, SMARCA4, SMARCB1, SMARCD1, SMARCEl, SMC1A, SMO, SND1, SNX29, SOCS1, SOX2, SOX21, SPECC1, SPEN, SPOP, SRC, SRGAP3, SRSF2, SRSF3, SS18, SS18L1, SSX1, SSX2, SSX4, STAG1, STAG2, STAT3, STAT5B, STAT6, STIL, STK11, STRN, SUFU, SUZ12, SYK, TAF15, TAL1, TAL2, TBL1XR1, TBX3, TCEA1, TCF12, TCF3, TCF7L2, TCL1A, TEC, TENT5C, TERT, TET1, TET2, TFE3, TFEB, TFG, TFPT, TFRC, TGFBR2, THRAP3, TLX1, TLX3, TMEM127, TMPRSS2, TNC, TNFAIP3, TNFRSF14, TNFRSF17, TOP1, TP53, TP63, TPM3, TPM4, TPR, TRA, TRAF7, TRB, TRD, TRIM24, TRIM27, TRIM33, TRIP11, TRRAP, TSC1, TSC2, TSHR, U2AF1, UBR5, USP44, USP6, USP8, VAV1, VHL, VTI1A, WAS, WDCP, WIF1, WNK2, WRN, WT1, WWTR1, XPA, XPC, XPO1, YWHAE, ZBTB16, ZCCHC8, ZEB1, ZFHX3, ZMYM2, ZMYM3, ZNF331, ZNF384, ZNF429, ZNF479, ZNF521, ZNRF3, or ZRSR2.

In some embodiments of any one of the populations of engineered cells disclosed herein, the artificially-induced modification is at least 0.5 kb, at least 1 kb, at least 2 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, at least 15 kb, at least 20 kb, at least 25 kb, at least 30 kb, at least 35 kb, at least 40 kb, at least 50 kb, at least 60 kb, at least 70 kb, at least 75 kb, at least 80 kb, at least 90 kb, at least 100 kb, at least 110 kb, at least 120 kb, at least 130 kb, at least 140 kb, at least 150 kb, at least 160 kb, at least 170 kb, at least 180 kb, at least 190 kb, at least 200 kb, at least 210 kb, at least 220 kb, at least 230 kb, at least 240 kb, at least 250 kb, at least 260 kb, at least 270 kb, at least 280 kb, at least 290 kb, or at least 300 kb from the nearest snoRNA-encoding, miRNA-encoding, or lincRNA-encoding gene in the genome.

In some embodiments of any one of the populations of engineered cells disclosed herein, the engineered cell is a stem cell (e.g., an isolated stem cell). In some embodiments of any one of the populations of engineered cells disclosed herein, the engineered cell is an embryonic stem cell. In some embodiments of any one of the populations of engineered cells disclosed herein, the engineered cell is an induced pluripotent stem cell (iPSC). In some embodiments of any one of the populations of engineered cells disclosed herein, the engineered cell is a multipotent stem cell. In some embodiments of any one of the populations of engineered cells disclosed herein, the engineered cell is a totipotent stem cell. In some embodiments of any one of the populations of engineered cells disclosed herein, the engineered cell is an immune cell. In some embodiments of any one of the populations of engineered cells disclosed herein, the engineered cell is an NK cell. In some embodiments of any one of the populations of engineered cells disclosed herein, the engineered cell is a T cell. In some embodiments of any one of the populations of engineered cells disclosed herein, the engineered cell is a mammalian cell. In some embodiments of any one of the populations of engineered cells disclosed herein, the engineered cell is a human cell.

In some embodiments of any one of the populations of engineered cells disclosed herein, the nearest 5′ open reading frame to the genomic site or the nearest 3′ open reading frame to the genomic site encodes a ribosomal protein. In some embodiments of any one of the populations of engineered cells disclosed herein, the nearest 5′ open reading frame to the genomic site or the nearest 3′ open reading frame to the genomic site encodes a ubiquitin family member. In some embodiments of any one of the populations of engineered cells disclosed herein, the nearest 5′ open reading frame to the genomic site or the nearest 3′ open reading frame to the genomic site encodes a ubiquitin modulator. In some embodiments of any one of the populations of engineered cells disclosed herein, the nearest 5′ open reading frame to the genomic site or the nearest 3′ open reading frame to the genomic site encodes a zinc finger-containing protein. In some embodiments of any one of the populations of engineered cells disclosed herein, the nearest 5′ open reading frame to the genomic site or the nearest 3′ open reading frame to the genomic site encodes a factor that positively regulates apoptosis. In some embodiments of any one of the populations of engineered cells disclosed herein, the nearest 5′ open reading frame to the genomic site or the nearest 3′ open reading frame to the genomic site encodes a factor that negatively regulates apoptosis. In some embodiments of any one of the populations of engineered cells disclosed herein, the nearest 5′ open reading frame to the genomic site or the nearest 3′ open reading frame to the genomic site encodes a cell cycle progression regulator. In some embodiments of any one of the populations of engineered cells disclosed herein, the nearest 5′ open reading frame to the genomic site or the nearest 3′ open reading frame to the genomic site encodes a transcription factor. In some embodiments of any one of the populations of engineered cells disclosed herein, the nearest 5′ open reading frame to the genomic site or the nearest 3′ open reading frame to the genomic site encodes a basic region/leucine zipper (bZIP) transcription factor. In some embodiments of any one of the populations of engineered cells disclosed herein, the nearest 5′ open reading frame to the genomic site or the nearest 3′ open reading frame to the genomic site encodes a DNA damage response regulator. In some embodiments of any one of the populations of engineered cells disclosed herein, the nearest 5′ open reading frame to the genomic site or the nearest 3′ open reading frame to the genomic site encodes a ubiquitin ligase.

In some embodiments of any one of the populations of engineered cells disclosed herein, the genomic site is not an adeno-associated virus integration site (AAVS). In some embodiments of any one of the populations of engineered cells disclosed herein, the genomic site is not AAVS1. In some embodiments of any one of the populations of engineered cells disclosed herein, the genomic site is not H11. In some embodiments of any one of the populations of engineered cells disclosed herein, the genomic site is not AAVS1 or H11. In some embodiments of any one of the populations of engineered cells disclosed herein, the genomic site is not Rosa26, colA1, TIGRE, or CCR5.

In some embodiments of any one of the populations of engineered cells disclosed herein, more than 95%, more than 95.1%, more than 95.2%, more than 95.3%, more than 95.4%, more than 95.5%, more than 95.6%, more than 95.7%, more than 95.8%, more than 95.9%, more than 96%, more than 96.1%, more than 96.2%, more than 96.3%, more than 96.4%, more than 96.5%, more than 96.6%, more than 96.7%, more than 96.8%, more than 96.9%, more than 97%, more than more than 97.1%, more than 97.2%, more than 97.3%, more than 97.4%, more than 97.5%, more than 97.6%, more than 97.7%, more than 97.8%, more than 97.9%, more than 98%, more than 98.1%, more than 98.2%, more than 98.3%, more than 98.4%, more than 98.5%, more than 98.6%, more than 98.7%, more than 98.8%, more than 98.9%, more than 99%, more than 99.1%, more than 99.2%, more than 99.3%, more than 99.4%, more than 99.5%, more than 99.6%, more than 99.7%, more than 99.8%, more than 99.85%, more than 99.9%, more than 99.55%, or more than 99.99% of the population maintain constitutive expression of the transgene for at least about 15 days. In some embodiments of any one of the populations of engineered cells disclosed herein, more than 95%, more than 95.1%, more than 95.2%, more than 95.3%, more than 95.4%, more than 95.5%, more than 95.6%, more than 95.7%, more than 95.8%, more than 95.9%, more than 96%, more than 96.1%, more than 96.2%, more than 96.3%, more than 96.4%, more than 96.5%, more than 96.6%, more than 96.7%, more than 96.8%, more than 96.9%, more than 97%, more than more than 97.1%, more than 97.2%, more than 97.3%, more than 97.4%, more than 97.5%, more than 97.6%, more than 97.7%, more than 97.8%, more than 97.9%, more than 98%, more than 98.1%, more than 98.2%, more than 98.3%, more than 98.4%, more than 98.5%, more than 98.6%, more than 98.7%, more than 98.8%, more than 98.9%, more than 99%, more than 99.1%, more than 99.2%, more than 99.3%, more than 99.4%, more than 99.5%, more than 99.6%, more than 99.7%, more than 99.8%, more than 99.85%, more than 99.9%, more than 99.55%, or more than 99.99% of the population can maintain constitutive expression of the transgene for at least about 21 days. In some embodiments of any one of the populations of engineered cells disclosed herein, more than 98.8% of the population maintains constitutive expression of the transgene for at least about 15 days.

In some aspects, the present disclosure provides a vector for generation of any one of the populations of engineered cells disclosed herein. The vector can comprise at least one homology arm. The homology arm can be at least 15, at least 20, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides in length. The homology arm can be at least 20 nucleotides in length. The homology arm can be at least 100 nucleotides in length. The homology arm can be at least 500 nucleotides in length. The homology arm can comprise a nucleotide sequence with at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.1%, at least 95.2%, at least 95.3%, at least 95.4%, at least 95.5%, at least 95.6%, at least 95.7%, at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.85%, at least 99.9%, at least 99.95%, or at least 99.99% sequence identity to a corresponding sequence in an intergenic region between: (a) FAU and ZNHIT2; (b) RPL3 and SYNGR1; (c) RPLP2 and PIDD1; (d) RPS7 and RNASEH1; (e) THEM4 and S100A10; (f) DDIT4 and ANAPC16; (g) ANXA2 and FOXB1; (h) TOB2 and TEF; (i) NDUFA4 and PHF14; (j) DDX5 and CEP95; (k) PIN4 and RPS4X; (l) PLEKHG2 and RPS16; (m) TRIM41 and RACK1; (n) HINT1 and LYRM7; (o) CFL1 and MUS81; or (p) VPS13B and COX6C. The homology arm can be at least 500 nucleotides in length. The homology arm can comprise a nucleotide sequence with at least 90% sequence identity to a corresponding sequence in an intergenic region between: (a) FAU and ZNHIT2; (b) RPL3 and SYNGR1; (c) RPLP2 and PIDD1; (d) RPS7 and RNASEH1; (e) THEM4 and S100A10; (f) DDIT4 and ANAPC16; (g) ANXA2 and FOXB1; (h) TOB2 and TEF; (i) NDUFA4 and PHF14; (j) DDX5 and CEP95; (k) PIN4 and RPS4X; (l) PLEKHG2 and RPS16; (m) TRIM41 and RACK1; (n) HINT1 and LYRM7; (o) CFL1 and MUS81; or (p) VPS13B and COX6C. The homology arm can be at least 500 nucleotides in length. The vector can comprise a second homology arm, for example, of a similar length as the first, and/or comprising a nucleotide sequence with high sequence identity to a second corresponding sequence that is adjacent to the first corresponding sequence in the genome.

In some aspects, the present disclosure provides a method of making any one of the populations of engineered cells disclosed herein. The method can comprise introducing the artificially-induced modification into the genomic site of a cell.

In some embodiments, the artificially-induced modification comprises an expression cassette, for example, for expression of a transgene. In some embodiments, introducing the artificially-induced modification comprises introducing a double-stranded break in the genomic site. In some embodiments, the double-stranded break is introduced by a nuclease. In some embodiments, the nuclease is a CRISPR-associated (Cas) nuclease, a transcription activator-like effector nuclease (TALEN) or a zinc finger nuclease. In some embodiments, introducing the artificially-induced modification comprises providing a polynucleotide to be integrated into the genomic site by homology-directed repair. In some embodiments, the polynucleotide to be integrated into the genomic site by homology-directed repair is present in a vector disclosed herein.

In some embodiments, after the introducing, silencing of expression of the transgene is observed in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90-fold, at least 100-fold, at least 200-fold, at least 300-fold, at least 400-fold, at least 500-fold, at least 600-fold, at least 700-fold, at least 800-fold, at least 900-fold, or at least 1000-fold fewer cells than a corresponding population of engineered cells with the transgene or expression cassette inserted at an AAVS1 locus. Determining the silencing of expression of the transgene can be done about 1, about 2, about 3, about 4, about 5, about 6, about 7, about 8, about 9, about 10, about 11, about 12, about 13, about 14, about 15, about 16, about 17, about 18, about 19, or about 20 weeks after introducing the artificially-induced modification into the genomic site. In some embodiments, 20 days after the introducing, silencing of expression of the transgene is observed in at least 1% fewer cells than a corresponding population of engineered cells with the transgene inserted at an AAVS1 locus. The percentages of cells with silencing can be determined by evaluating at least five, at least ten, at least twenty, at least 30, at least 40, at least 50, at least 60, at least 70, at least 80, at least 90, or at least 100 clones with the transgene inserted at the AAVS1 locus and a similar or same number of clones with the transgene inserted at the genomic site. The percentages of cells with silencing can be determined by evaluating at least ten clones with the transgene inserted at the genomic site and at least ten clones with the transgene inserted at the AAVS1 locus.

In some embodiments, after the introducing, expression of the transgene persists for at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 2-fold, at least 3-fold, at least 4-fold, at least 5-fold, at least 6-fold, at least 7-fold, at least 8-fold, at least 9-fold, or at least 10-fold longer than a corresponding population of engineered cells with the transgene inserted at an AAVS1 locus. The duration of transgene expression can be determined by evaluating at least five, at least ten, at least twenty, at least 30, at least 40, at least 50, at least 60, at least 70, at least 80, at least 90, or at least 100 clones with the transgene inserted at the AAVS1 locus and a similar or same number of clones with the transgene inserted at the genomic site. The duration of transgene expression can be determined by evaluating at least ten clones with the transgene inserted at the genomic site and at least ten clones with the transgene inserted at the AAVS1 locus. The duration of transgene expression can be evaluated by determining the first measured time point when at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the engineered cells no longer express the transgene. The duration of transgene expression can be evaluated by determining the first measured time point when at least 5% of the engineered cells no longer express the transgene. The duration of transgene expression can be evaluated by determining the first measured time point when at least 10% of the engineered cells no longer express the transgene. The duration of transgene expression can be evaluated by determining the first measured time point when at least 20% of the engineered cells no longer express the transgene.

In some aspects, the present disclosure provides a pharmaceutical composition comprising any one of the populations of engineered cells disclosed herein, and a pharmaceutically-acceptable excipient, carrier, vehicle, or diluent.

In some aspects, the present disclosure provides a pharmaceutical composition comprising any one of the vectors disclosed herein, and a pharmaceutically-acceptable excipient, carrier, vehicle, or diluent.

In some aspects, the present disclosure provides a method of treating a condition in a subject in need thereof, the method comprising administering to the subject any one of the populations of engineered cells disclosed herein. The population of engineered cells can be present in a pharmaceutical composition disclosed herein.

In some aspects, the present disclosure provides a method of treating a condition in a subject in need thereof, the method comprising administering to the subject any one of the vectors disclosed herein. The vector can be present in a pharmaceutical composition disclosed herein.

In some embodiments, the condition is acute myeloid leukemia (AML). In some embodiments, the condition is multiple myeloma (MM). In some embodiments, the condition is Myelodysplastic syndrome (MDS). In some embodiments, the condition is B cell leukemia. In some embodiments, the condition is T cell leukemia. In some embodiments, the condition is a solid tumor. In some embodiments, the condition is a blood cancer.

### III. ADDITIONAL ASPECTS OF THE SAFE HARBOR LOCI AND ENGINEERED CELLS

### A. Stability of Expression

Safe harbor loci of the disclosure can support stable and sustained expression of transgenes of the disclosure. As demonstrated herein, other safe harbor loci are prone to silencing, with at least some clones losing transgene expression in a proportion of cells, e.g., after several passages in culture.

In cases where a transgene is operatively coupled to a constitutive promoter, maintaining expression can generally refer to maintaining a detectable level of expression in live cells. In cases where a transgene is operatively coupled to an inducible promoter, maintaining expression can generally refer to maintaining a capability to induce a detectable level of expression in live cells with an appropriate stimulus. In cases where a transgene is operatively coupled to a tissue-specific promoter, maintaining expression can generally refer to maintaining a capability of expressing a detectable level of the transgene in live cells in an appropriate regulatory context, for example, in the presence of transcription factors and/or other regulatory elements that induce expression from the tissue-specific promoter. Expression of the transgene can be measured using any appropriate method in engineered cells of the disclosure, e.g., qPCR, RNAseq, gene arrays, ELISA, flow cytometry, mass cytometry, etc.

In some embodiments, maintenance of expression of a transgene that is present in a safe harbor locus of the disclosure can be determined by evaluating any one of the populations of engineered cells disclosed herein about 5, about 6, about 7, about 8, about 9, about 10, about 11, about 12, about 13, about 14, about 15, about 16, about 17, about 18, about 19, about 20, about 21, about 25, about 30, about 35, about 40, about 45, about 50, about 55, about 60, about 65, about 70, about 75, about 80, about 85, about 90, about 95, about 100, about 110, about 120, about 130, about 140, about 150, about 100, about 200, about 300, or about 365 days after the transgene is introduced into the genomic site.

In some embodiments, maintenance of expression of a transgene that is present in a safe harbor locus of the disclosure can be determined by evaluating any one of the populations of engineered cells disclosed herein at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 100, at least about 200, at least about 300, or at least about 365 days after the transgene is introduced into the genomic site.

In some embodiments, maintenance of expression of a transgene can be determined for a population of cells that are subjected to differentiation towards a cell lineage, or after differentiation into a particular cell lineage or specific cell type. Maintenance of expression of a transgene that is present in a safe harbor locus of the disclosure can be determined by evaluating the population of cells about 5, about 6, about 7, about 8, about 9, about 10, about 11, about 12, about 13, about 14, about 15, about 16, about 17, about 18, about 19, about 20, about 21, about 25, about 30, about 35, about 40, about 45, about 50, about 55, about 60, about 65, about 70, about 75, about 80, about 85, about 90, about 95, about 100, about 110, about 120, about 130, about 140, about 150, about 100, about 200, about 300, or about 365 days after inducing differentiation towards the cell lineage or specific cell type.

Maintenance of expression of a transgene that is present in a safe harbor locus of the disclosure can be determined by evaluating the population of cells at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 100, at least about 200, at least about 300, or at least about 365 days after inducing differentiation towards the cell lineage or specific cell type.

In some embodiments, maintenance of expression of a transgene that is present in a safe harbor locus of the disclosure can be determined by evaluating any one of the populations of engineered cells disclosed herein about 3, about 4, about 5, about 6, about 7, about 8, about 9, about 10, about 11, about 12, about 13, about 14, about 15, about 16, about 17, about 18, about 19, about 20, about 25, about 30, about 40, about 50, about 60, about 70, about 80, about 90, or about 100 passages after the transgene is introduced into the genomic site.

In some embodiments, maintenance of expression of a transgene that is present in a safe harbor locus of the disclosure can be determined by evaluating any one of the populations of engineered cells disclosed herein at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 40, at least about 50, at least about 60, at least about 70, at least about 80, at least about 90, or at least about 100 passages after the transgene is introduced into the genomic site. A passage can be, for example, about 2-4 days, such as about 2, about 3, or about 4 days, or any other length of time as appropriate for culturing the particular engineered cell type.

In some embodiments, maintenance of expression of a transgene that is present in a safe harbor locus of the disclosure can be determined by evaluating any one of the populations of engineered cells disclosed herein at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 100, at least about 200, at least about 300, or at least about 365 days after the transgene is introduced into the genomic site.

In some embodiments, more than 20%, more than 25%, more than 30%, more than 35%, more than 40%, more than 45%, more than 50%, more than 55%, more than 60%, more than 61%, more than 62%, more than 63%, more than 64%, more than 65%, more than 66%, more than 67%, more than 68%, more than 69%, more than 70%, more than 71%, more than 72%, more than 73%, more than 74%, more than 75%, more than 76%, more than 77%, more than 78%, more than 79%, more than 80%, more than 81%, more than 82%, more than 83%, more than 84%, more than 85%, more than 86%, more than 87%, more than 88%, more than 89%, more than 90%, more than 91%, more than 92%, more than 93%, more than 94%, more than 95%, more than 95.1%, more than 95.2%, more than 95.3%, more than 95.4%, more than 95.5%, more than 95.6%, more than 95.7%, more than 95.8%, more than 95.9%, more than 96%, more than 96.1%, more than 96.2%, more than 96.3%, more than 96.4%, more than 96.5%, more than 96.6%, more than 96.7%, more than 96.8%, more than 96.9%, more than 97%, more than more than 97.1%, more than 97.2%, more than 97.3%, more than 97.4%, more than 97.5%, more than 97.6%, more than 97.7%, more than 97.8%, more than 97.9%, more than 98%, more than 98.1%, more than 98.2%, more than 98.3%, more than 98.4%, more than 98.5%, more than 98.6%, more than 98.7%, more than 98.8%, more than 98.9%, more than 99%, more than 99.1%, more than 99.2%, more than 99.3%, more than 99.4%, more than 99.5%, more than 99.6%, more than 99.7%, more than 99.8%, more than 99.85%, more than 99.9%, more than 99.95%, or more than 99.99% of the population can maintain expression of the transgene for at least a length of time disclosed herein (for example, about 15 days, about 21 days, about 2 months, about 3 months, about 6 months, or about a year).

In some embodiments, the percentage of cells that maintain expression of a transgene of the disclosure can exhibit heterogeneity between clones. For example, in some examples disclosed herein, several clones are each grown up from single cells that each comprise the same genomic modification, and some clones retain expression of the transgene in a high percentage of cells, while other clones exhibit considerably lower maintenance of expression. To account for such heterogeneity, in some embodiments multiple clones are evaluated. For example, in some embodiments, at least five, at least ten, at least 20, at least 30, at least 40, at least 50, at least 60, at least 70, at least 80, at least 90, or at least 100 clones with the same genetic modification can be evaluated. The results can be averaged, or, for example, a proportion of clones that maintain expression above a certain threshold percentage of cells can be determined. The same method can be used to compare maintenance of expression between safe harbor loci, for example, between a safe harbor locus of the disclosure and a control safe harbor locus, such as AAVS1 or H11.

In some embodiments, (i) a percentage of cells expressing the transgene from a plurality of clones comprising the transgene inserted at a genomic site of the disclosure is higher than (ii) a percentage of cells expressing the transgene from a plurality of clones comprising the transgene inserted at an AAVS1 locus. The clones can be evaluated any suitable period of time disclosed herein subsequent to introducing the transgene, for example, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 100, at least about 200, at least about 300, or at least about 365 days after the transgene is introduced into the genomic site. In some embodiments, (i) is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, or at least 80% higher than (ii).

In some embodiments, (i) a duration of expression of the transgene from a plurality of clones comprising the transgene inserted at the genomic site is greater than (ii) a duration of expression of the transgene from a plurality of clones comprising the transgene inserted at an AAVS1 locus. In some embodiments, (i) is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90-fold, or at least 100-fold greater than (ii).

In some embodiments, (i) an average expression level of the transgene from a plurality of clones comprising the transgene inserted at the genomic site is higher than (ii) an average expression level of the transgene from a plurality of clones comprising the transgene inserted at an AAVS1 locus. An average expression level can be determined by any suitable technique, for example, average (e.g., mean, geometric mean, median) fluorescence intensity, qPCT, RNAseq, ELISA, western blot, etc. In some embodiments, (i) is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90-fold, or at least 100-fold higher than (ii).

In some embodiments, testing how well a prospective safe harbor locus supports maintained expression of a transgene can comprise determining a time point when at least 5%, at least 10%, at least 15%, at least 20%, at least 25%, at least 30%, at least 35%, at least 40%, at least 45%, at least 50%, at least 55%, at least 60%, at least 65%, at least 70%, or at least 75% of clones no longer express the transgene in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the engineered cells of the clone.

In some embodiments, testing how well a prospective safe harbor locus supports maintained expression of a transgene can comprise determining a time point when at least 25% of clones no longer express the transgene in at least 98% of the engineered cells of the clone. In some embodiments, testing how well a prospective safe harbor locus supports maintained expression of a transgene can comprise determining a time point when at least 50% of clones no longer express the transgene in at least 98% of the engineered cells of the clone. In some embodiments, testing how well a prospective safe harbor locus supports maintained expression of a transgene can comprise determining a time point when at least 75% of clones no longer express the transgene in at least 98% of the engineered cells of the clone.

In some embodiments, testing how well a prospective safe harbor locus supports maintained expression of a transgene can comprise determining a time point when at least 25% of clones no longer express the transgene in at least 95% of the engineered cells of the clone. In some embodiments, testing how well a prospective safe harbor locus supports maintained expression of a transgene can comprise determining a time point when at least 50% of clones no longer express the transgene in at least 95% of the engineered cells of the clone. In some embodiments, testing how well a prospective safe harbor locus supports maintained expression of a transgene can comprise determining a time point when at least 75% of clones no longer express the transgene in at least 95% of the engineered cells of the clone.

In some embodiments, testing how well a prospective safe harbor locus supports maintained expression of a transgene can comprise determining a time point when at least 25% of clones no longer express the transgene in at least 90% of the engineered cells of the clone. In some embodiments, testing how well a prospective safe harbor locus supports maintained expression of a transgene can comprise determining a time point when at least 50% of clones no longer express the transgene in at least 90% of the engineered cells of the clone. In some embodiments, testing how well a prospective safe harbor locus supports maintained expression of a transgene can comprise determining a time point when at least 75% of clones no longer express the transgene in at least 90% of the engineered cells of the clone.

In some embodiments, testing how well a prospective safe harbor locus supports maintained expression of a transgene can comprise determining a time point when at least 25% of clones no longer express the transgene in at least 80% of the engineered cells of the clone. In some embodiments, testing how well a prospective safe harbor locus supports maintained expression of a transgene can comprise determining a time point when at least 50% of clones no longer express the transgene in at least 80% of the engineered cells of the clone. In some embodiments, testing how well a prospective safe harbor locus supports maintained expression of a transgene can comprise determining a time point when at least 75% of clones no longer express the transgene in at least 80% of the engineered cells of the clone.

In some embodiments, testing how well a prospective safe harbor locus supports maintained expression of a transgene can comprise determining a proportion of clones that no longer express the transgene in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the engineered cells after a time period disclosed herein (for example, about 15 days, about 21 days, about 2 months, about 3 months, about 6 months, or about a year).

### B. Genomic Sites

In some embodiments, the disclosure provides genomic sites that are safe harbors, and are suitable as sites for artificially-induced modification, for example, insertion of expression cassettes for expression of transgenes disclosed herein. Certain characteristics of the genetic context of the safe harbor loci are also disclosed herein, such as adjacent genes and classes thereof in the 5′ and/or 3′ direction, and distances from open reading frames, cancer-associated genes, snoRNA-encoding, miRNA-encoding, and lincRNA-encoding genes.

In some embodiments, the nearest open reading frame in a 5′ direction or the nearest open reading frame in a 3′ direction to any one of the genomic sites disclosed herein encodes a ribosomal protein, for example, a protein that forms part of or interacts with a ribosomal subunit, or contributes to ribosome biogenesis. In some embodiments, the nearest open reading frame to any one of the genomic sites disclosed herein encodes a ribosomal protein, for example, a protein that forms part of or interacts with a ribosomal subunit, or contributes to ribosome biogenesis. Non-limiting examples of ribosomal proteins include FAU, ZNHIT2, RPS7, RPLP2, RPL3, RPS4X, RPS16, and PIN4.

In some embodiments, the nearest open reading frame in a 5′ direction or the nearest open reading frame in a 3′ direction to any one of the genomic sites disclosed herein encodes a ubiquitin modulator, for example, a ubiquitin ligase, or a protein that contributes to mono or polyubiquitination (e.g., K48 or K63 ubiquitination). In some embodiments, the nearest open reading frame to any one of the genomic sites disclosed herein encodes a ubiquitin modulator. Non-limiting examples of ubiquitin modulators include FAU, PIDD1, ANAPC16.

In some embodiments, the nearest open reading frame in a 5′ direction or the nearest open reading frame in a 3′ direction to any one of the genomic sites disclosed herein encodes an apoptosis regulator, for example, a positive or negative regulator of apoptosis. In some embodiments, the nearest open reading frame to any one of the genomic sites disclosed herein encodes an apoptosis regulator. Non-limiting examples of apoptosis regulators include PIDD1, DDIT4, and TOB2.

In some embodiments, the nearest open reading frame in a 5′ direction or the nearest open reading frame in a 3′ direction to any one of the genomic sites disclosed herein encodes a cell cycle progression regulator, for example, a factor that promotes or inhibits cell cycle progression. In some embodiments, the nearest open reading frame to any one of the genomic sites disclosed herein encodes a cell cycle progression regulator. Non-limiting examples of cell cycle progression regulators include DDIT4, ANAPC16, TOB2, and PIN4.

In some embodiments, the nearest open reading frame in a 5′ direction or the nearest open reading frame in a 3′ direction to any one of the genomic sites disclosed herein encodes a transcription factor. In some embodiments, the nearest open reading frame to any one of the genomic sites disclosed herein encodes a transcription factor. In some embodiments the transcription factor is TEF.

In some embodiments, the nearest open reading frame in a 5′ direction or the nearest open reading frame in a 3′ direction to any one of the genomic sites disclosed herein encodes a zinc finger-containing protein. In some embodiments, the nearest open reading frame to any one of the genomic sites disclosed herein encodes a zinc finger-containing protein. Non-limiting examples of zinc finger-containing proteins include ZNHIT2.

In some embodiments, the nearest open reading frame in a 5′ direction or the nearest open reading frame in a 3′ direction to any one of the genomic sites disclosed herein encodes a basic region/leucine zipper (bZIP) transcription factor. In some embodiments, the nearest open reading frame to any one of the genomic sites disclosed herein encodes a basic region/leucine zipper (bZIP) transcription factor. TEF is a non-limiting example of a basic region/leucine zipper (bZIP) transcription factor.

In some embodiments, the nearest open reading frame in a 5′ direction or the nearest open reading frame in a 3′ direction to any one of the genomic sites disclosed herein encodes a DNA damage response regulator. In some embodiments, the nearest open reading frame to any one of the genomic sites disclosed herein encodes a DNA damage response regulator. Non-limiting examples of DNA damage response regulators include PIDD1, DDIT4, and MUS81.

In some embodiments, the nearest open reading frame in a 5′ direction or the nearest open reading frame in a 3′ direction to any one of the genomic sites disclosed herein encodes a ubiquitin ligase. In some embodiments, the nearest open reading frame to any one of the genomic sites disclosed herein encodes a ubiquitin ligase. Non-limiting examples of ubiquitin ligases include AFF4, AMFR, ANAPC11, ANAPC16, ANKIB1, APC/C, AREL1, ARIH1, ARIH2, BARD1, beta-TrCP1, BFAR, BIRC2, BIRC3, BIRC7, BIRC8, BMI1, BRAP, BRCA1, c-IAP1CBL, CBLB, CBLC, CBLL1, CCDC36, CCNB1IP1, Cereblon (CRBN), CGRRF1, CHFR, CHIP, CNOT4, CUL9, CYHR1, DCST1, DTX1, DTX2, DTX3, DTX3L, DTX4, DZIP3, E4F1, E6AP, FANCL, G2E3, gp78, HACE1, HECTD1, HECTD2, HECTD3, HECTD4, HECW1, HECW2, HERC1, HERC2, HERC3, HERC4, HERC5, HERC6, HLTF, HOIL-IL, HOIP, HUL5, HUWE1, IAP, IRF2BP1, IRF2BP2, IRF2BPL, Itch, KCMF1, KMT2C, KMT2D, LNX1, LNX2, LONRF1, LONRF2, LONRF3, LRSAM1, LTN1, LUBAC, MAEA, MAP3K1, MARCH1, MARCH10, MARCH11, MARCH2, MARCH3, MARCH4, MARCH5, MARCH6, MARCH7, MARCH8, MARCH9, Mdm2, MDM4, MECOM, MEX3A, MEX3B, MEX3C, MEX3D, MGRN1, MIB1, MIB2, MID1, MID2, MKRN1, MKRN2, MKRN3, MKRN4P, MNAT1, MSL2, MUL1, MYCBP2, MYLIP, NEDD4, NEDD4L, NEURL1, NEURL1B, NEURL3, NFX1, NFXL1, NHLRC1, NOSIP, NSMCE1, Parkin, PARK2, PCGF1, PCGF2, PCGF3, PCGF5, PCGF6, PDZRN3, PDZRN4, PELI1, PELI2, PELI3, PEX10, PEX12, PEX2, PHF7, PHRF1, PJA1, PJA2, PLAG1, PLAGL1, PML, PPIL2, PRPF19, pVHL, RAD18, RAG1, RAPSN, RBBP6, RBCK1, RBX1, RC3H1, RC3H2, RCHY1, RFFL, RFPL1, RFPL2, RFPL3, RFPL4A, RFPL4AL1, RFPL4B, RFWD2, RFWD3, RING1, RLF, RLIM, RMND5A, RMND5B, RNF10, RNF103, RNF11, RNF111, RNF112, RNF113A, RNF113B, RNF114, RNF115, RNF121, RNF122, RNF123, RNF125, RNF126, RNF128, RNF13, RNF130, RNF133, RNF135, RNF138, RNF139, RNF14, RNF141, RNF144A, RNF144B, RNF145, RNF146, RNF148, RNF149, RNF150, RNF151, RNF152, RNF157, RNF165, RNF166, RNF167, RNF168, RNF169, RNF17, RNF170, RNF175, RNF180, RNF181, RNF182, RNF183, RNF185, RNF186, RNF187, RNF19A, RNF19B, RNF2, RNF20, RNF207, RNF208, RNF212, RNF212B, RNF213, RNF214, RNF215, RNF216, RNF217, RNF219, RNF220, RNF222, RNF223, RNF224, RNF225, RNF24, RNF25, RNF26, RNF31, RNF32, RNF34, RNF38, RNF39, RNF4, RNF40, RNF41, RNF43, RNF44, RNF5, RNF6, RNF7, RNF8, RNFT1, RNFT2, Rsp5, RSPRY1, San1, SCAF11, SCF, SHARPIN, SH3RF1, SH3RF2, SH3RF3, SHPRH, SIAH1, SIAH2, SIAH3, SMURF1, SMURF2, STUB1, SYVN1, TMEM129, Topors, TRAF2, TRAF3, TRAF4, TRAF5, TRAF6, TRAF7, TRAIP, TRIM10, TRIM11, TRIM13, TRIM15, TRIM17, TRIM2, TRIM21, TRIM22, TRIM23, TRIM24, TRIM25, TRIM26, TRIM27, TRIM28, TRIM3, TRIM31, TRIM32, TRIM33, TRIM34, TRIM35, TRIM36, TRIM37, TRIM38, TRIM39, TRIM4, TRIM40, TRIM41, TRIM42, TRIM43, TRIM43B, TRIM45, TRIM46, TRIM47, TRIM48, TRIM49, TRIM49B, TRIM49C, TRIM49D1, TRIM5, TRIM50, TRIM51, TRIM52, TRIM54, TRIM55, TRIM56, TRIM58, TRIM59, TRIM6, TRIM60, TRIM61, TRIM62, TRIM63, TRIM64, TRIM64B, TRIM64C, TRIM65, TRIM67, TRIM68, TRIM69, TRIM7, TRIM71, TRIM72, TRIM73, TRIM74, TRIM75P, TRIM77, TRIM8, TRIM9, TRIML1, TRIML2, TRIP12, TTC3, UBE3A, UBE3B, UBE3C, UBE3D, UBE4A, UBE4B, UBOX5, UBR1, UBR2, UBR3, UBR4, UBR5, UBR7, UHRF1, UHRF2, UNK, UNKL, VHL, VPS11, VPS18, VPS41, VPS8, WDR59, WDSUB1, WWP1, WWP2, XIAP, ZBTB12, ZFP91, ZFPL1, ZNF280A, ZNF341, ZNF511, ZNF521, ZNF598, ZNF645, ZNRF1, ZNRF2, ZNRF3, ZNRF4, Zswim2, and ZXDC (which are also ubiquitin modulators). In some embodiments, the ubiquitin ligase is ANAPC16.

In some embodiments, any one of the genomic sites disclosed herein is adjacent to a promoter operatively coupled to FAU. In some embodiments, any one of the genomic sites disclosed herein is adjacent to a promoter operatively coupled to ZNHIT2. In some embodiments, any one of the genomic sites disclosed herein is adjacent to a promoter operatively coupled to RPL3. In some embodiments, any one of the genomic sites disclosed herein is adjacent to a promoter operatively coupled to RPLP2. In some embodiments, any one of the genomic sites disclosed herein is adjacent to a promoter operatively coupled to RPS7. In some embodiments, any one of the genomic sites disclosed herein is adjacent to a promoter operatively coupled to TMEM4. In some embodiments, any one of the genomic sites disclosed herein is adjacent to a promoter operatively coupled to S100A10. In some embodiments, any one of the genomic sites disclosed herein is adjacent to a promoter operatively coupled to ANAPC16. In some embodiments, any one of the genomic sites disclosed herein is adjacent to a promoter operatively coupled to DDIT4. In some embodiments, any one of the genomic sites disclosed herein is adjacent to a promoter operatively coupled to FOXB1. In some embodiments, any one of the genomic sites disclosed herein is adjacent to a promoter operatively coupled to ANXA2. In some embodiments, any one of the genomic sites disclosed herein is adjacent to a promoter operatively coupled to TEF. In some embodiments, any one of the genomic sites disclosed herein is adjacent to a promoter operatively coupled to TOB2. In some embodiments, any one of the genomic sites disclosed herein is adjacent to a promoter operatively coupled to NDUFA4. In some embodiments, any one of the genomic sites disclosed herein is adjacent to a promoter operatively coupled to DDX5. In some embodiments, any one of the genomic sites disclosed herein is adjacent to a promoter operatively coupled to CEP95. In some embodiments, any one of the genomic sites disclosed herein is adjacent to a promoter operatively coupled to PIN4. In some embodiments, any one of the genomic sites disclosed herein is adjacent to a promoter operatively coupled to RPS4X. In some embodiments, any one of the genomic sites disclosed herein is adjacent to a promoter operatively coupled to PLEKHG2. In some embodiments, any one of the genomic sites disclosed herein is adjacent to a promoter operatively coupled to RPS16. In some embodiments, any one of the genomic sites disclosed herein is adjacent to a promoter operatively coupled to TRIM41. In some embodiments, any one of the genomic sites disclosed herein is adjacent to a promoter operatively coupled to RACK1. In some embodiments, any one of the genomic sites disclosed herein is adjacent to a promoter operatively coupled to HINT1. In some embodiments, any one of the genomic sites disclosed herein is adjacent to a promoter operatively coupled to CFL1. In some embodiments, any one of the genomic sites disclosed herein is adjacent to a promoter operatively coupled to MUS81. In some embodiments, any one of the genomic sites disclosed herein is adjacent to a promoter operatively coupled to VPS13B. In some embodiments, any one of the genomic sites disclosed herein is adjacent to a promoter operatively coupled to and COX6C.

In some embodiments, the genomic site is or is within an intergenic region between FAU and ZNHIT2. In some embodiments, the genomic site is or is within an intergenic region between RPL3 and SYNGR1. In some embodiments, the genomic site is or is within an intergenic region between RPLP2 and PIDD1. In some embodiments, the genomic site is or is within an intergenic region between RPS7 and RNASEH1. In some embodiments, the genomic site is or is within an intergenic region between THEM4 and S100A10. In some embodiments, the genomic site is or is within an intergenic region between DDIT4 and ANAPC16. In some embodiments, the genomic site is or is within an intergenic region between ANXA2 and FOXB1. In some embodiments, the genomic site is or is within an intergenic region between TOB2 and TEF. In some embodiments, the genomic site is or is within an intergenic region between NDUFA4 and PHF14. In some embodiments, the genomic site is or is within an intergenic region between DDX5 and CEP95. In some embodiments, the genomic site is or is within an intergenic region between PIN4 and RPS4X. In some embodiments, the genomic site is or is within an intergenic region between PLEKHG2 and RPS16. In some embodiments, the genomic site is or is within an intergenic region between TRIM41 and RACK1. In some embodiments, the genomic site is or is within an intergenic region between HINT1 and LYRM7. In some embodiments, the genomic site is or is within an intergenic region between CFL1 and MUS81. In some embodiments, the genomic site is or is within an intergenic region between VPS13B and COX6C.

In some embodiments, of any one of genomic sites disclosed herein has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.1%, at least 95.2%, at least 95.3%, at least 95.4%, at least 95.5%, at least 95.6%, at least 95.7%, at least 95.8%, at least 95.9%, at least 96%, at least 96.1%, at least 96.2%, at least 96.3%, at least 96.4%, at least 96.5%, at least 96.6%, at least 96.7%, at least 96.8%, at least 96.9%, at least 97%, at least 97.1%, at least 97.2%, at least 97.3%, at least 97.4%, at least 97.5%, at least 97.6%, at least 97.7%, at least 97.8%, at least 97.9%, at least 98%, at least 98.1%, at least 98.2%, at least 98.3%, at least 98.4%, at least 98.5%, at least 98.6%, at least 98.7%, at least 98.8%, at least 98.9%, at least 99%, at least 99.1%, at least 99.2%, at least 99.3%, at least 99.4%, at least 99.5%, at least 99.6%, at least 99.7%, at least 99.8%, at least 99.85%, at least 99.9%, at least 99.95%, or at least 99.99% sequence identity to a sequence from the human genome selected from the group consisting of: (a) chr11:65,117,969-65,120,057; (b) chr22:39,319,072-39,321,167; (c) chr11:808,403-810,414; (d) chr2:3,574,031-3,576,263; (e) chr1:151,944,637-151,946,598; (f) chr10:72,259,705-72,261,554; (g) chr15:60,126,969-60,128,831; (h) chr22:41,413,106-41,414,808; (i) chr7:10,940,150-10,940,760; (j) chr17:64,506,290-64,506,960; (k) chrX:72,268,950-72,270,750; (l) chr19:39,430,700-39,431,400; (m) chr5:181,235,790-181,236,860; (n) chr5:131,165,330-131,165,510; (o) chr11:65,859,410-65,860,050; and (p) chr8:99,877,580-99,877,850 of, for example, Genome Reference Consortium Human Build 38 (GRCh38/hg38).

In some embodiments, of any one of genomic sites disclosed herein is within a set of coordinates from the human genome selected from the group consisting of: (a) chr11:65,117,969-65,120,057; (b) chr22:39,319,072-39,321,167; (c) chr11:808,403-810,414; (d) chr2:3,574,031-3,576,263; (e) chr1:151,944,637-151,946,598; (f) chr10:72,259,705-72,261,554; (g) chr15:60,126,969-60,128,831; (h) chr22:41,413,106-41,414,808; (i) chr7:10,940,150-10,940,760; (j) chr17:64,506,290-64,506,960; (k) chrX:72,268,950-72,270,750; (l) chr19:39,430,700-39,431,400; (m) chr5:181,235,790-181,236,860; (n) chr5:131,165,330-131,165,510; (o) chr11:65,859,410-65,860,050; and (p) chr8:99,877,580-99,877,850 of, for example, Genome Reference Consortium Human Build 38 (GRCh38/hg38). In some embodiments, the genomic site is within the set of coordinates chr11:65,117,969-65,120,057. In some embodiments, the genomic site is within the set of coordinates chr22:39,319,072-39,321,167. In some embodiments, the genomic site is within the set of coordinates chr11:808,403-810,414. In some embodiments, the genomic site is within the set of coordinates chr2:3,574,031-3,576,263. In some embodiments, the genomic site is within the set of coordinates chr1:151,944,637-151,946,598. In some embodiments, the genomic site is within the set of coordinates chr10:72,259,705-72,261,554. In some embodiments, the genomic site is within the set of coordinates chr15:60,126,969-60,128,831. In some embodiments, the genomic site is within the set of coordinates chr22:41,413,106-41,414,808. In some embodiments, the genomic site is within the set of coordinates chr7:10,940,150-10,940,760. In some embodiments, the genomic site is within the set of coordinates chr17:64,506,290-64,506,960. In some embodiments, the genomic site is within the set of coordinates chrX:72,268,950-72,270,750. In some embodiments, the genomic site is within the set of coordinates chr19:39,430,700-39,431,400. In some embodiments, the genomic site is within the set of coordinates chr5:181,235,790-181,236,860. In some embodiments, the genomic site is within the set of coordinates chr5:131,165,330-131,165,510. In some embodiments, the genomic site is within the set of coordinates chr11:65,859,410-65,860,050. In some embodiments, the genomic site is within the set of coordinates chr8:99,877,580-99,877,850.

In some embodiments a genomic site is characterized by two or more, three or more, four or more, five or more, or six members selected from the group consisting of: (a) distance from the nearest open reading frame in the genome; (b) distance from the nearest cancer-associated gene in the genome; (c) distance from the nearest snoRNA-encoding, miRNA-encoding, or lincRNA-encoding gene in the genome; (d) not within a gene transcription unit; (e) not within an ultra-conserved region; (f) not within a VISTA enhancer region; and (g) within a DNase hypersensitive site.

In some embodiments, distance from the nearest open reading frame in the genome is at least 0.5 kb, at least 1 kb, at least 2 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, or at least 15 kb from the nearest open reading frame in the genome.

In some embodiments, distance from the nearest cancer-associated gene in the genome is at least 0.5 kb, at least 1 kb, at least 2 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, at least 15 kb, at least 20 kb, at least 25 kb, at least 30 kb, at least 35 kb, at least 40 kb, at least 50 kb, at least 60 kb, at least 70 kb, at least 75 kb, at least 80 kb, at least 90 kb, at least 100 kb, at least 110 kb, at least 120 kb, at least 130 kb, at least 140 kb, at least 150 kb, at least 160 kb, at least 170 kb, at least 180 kb, at least 190 kb, at least 200 kb, at least 210 kb, at least 220 kb, at least 230 kb, at least 240 kb, at least 250 kb, at least 260 kb, at least 270 kb, at least 280 kb, at least 290 kb, or at least 300 kb from the nearest cancer-associated gene in the genome.

In some embodiments, distance from the nearest snoRNA-encoding, miRNA-encoding, or lincRNA-encoding gene in the genome is at least 0.5 kb, at least 1 kb, at least 2 kb, at least 3 kb, at least 4 kb, at least 5 kb, at least 6 kb, at least 7 kb, at least 8 kb, at least 9 kb, at least 10 kb, at least 11 kb, at least 12 kb, at least 13 kb, at least 14 kb, at least 15 kb, at least 20 kb, at least 25 kb, at least 30 kb, at least 35 kb, at least 40 kb, at least 50 kb, at least 60 kb, at least 70 kb, at least 75 kb, at least 80 kb, at least 90 kb, at least 100 kb, at least 110 kb, at least 120 kb, at least 130 kb, at least 140 kb, at least 150 kb, at least 160 kb, at least 170 kb, at least 180 kb, at least 190 kb, at least 200 kb, at least 210 kb, at least 220 kb, at least 230 kb, at least 240 kb, at least 250 kb, at least 260 kb, at least 270 kb, at least 280 kb, at least 290 kb, or at least 300 kb from the nearest snoRNA-encoding, miRNA-encoding, or lincRNA-encoding gene in the genome.

In some embodiments a genomic site is characterized by two or more, three or more, four or more, five or more, or six members selected from the group consisting of: (a) at least 6 kb away from the nearest open reading frame in the genome; (b) at least 20 kb away from the nearest cancer-associated gene in the genome; (c) at least 20 kb away from the nearest snoRNA-encoding, miRNA-encoding, or lincRNA-encoding gene in the genome; (d) not within a gene transcription unit; (e) not within an ultra-conserved region; (f) not within a VISTA enhancer region; and (g) within a DNase hypersensitive site.

### C. Off Target Effects

A genomic site (e.g., safe harbor locus) of the disclosure can be used as a site for an artificially-introduced modification in the genome, wherein the artificially-introduced modification has minimal off-target effects, for example, minimal unintended impacts on cellular functions. The artificially-induced modification can comprise, for example, integration of an expression cassette for expression of a transgene.

In some embodiments, the artificially-introduced modification has minimal impact on cellular functions as determined by functional assays. Non-limiting examples of functional assays include proliferation assays, differentiation assays, migration assays, cytotoxicity assays (e.g., ability of engineered immune cells to kill target cells), assays evaluating cytokine production in response to a stimulus (e.g., pathogen-associated molecular patterns), differentiation assays (e.g., the ability to differentiate a stem cell or a precursor cell into a particular lineage, or a committed or terminally-differentiated cell type) and assays evaluating response to pro-apoptotic stimuli.

In some embodiments, the artificially-introduced modification has minimal impact on global gene expression, for example, as determined by RNA seq or a gene array.

In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 10 endogenous genes.

In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 50 endogenous genes.

In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 100 endogenous genes.

In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 200 endogenous genes.

In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 300 endogenous genes.

In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 500 endogenous genes.

In some embodiments, the artificially-induced modification effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 1000 endogenous genes.

In some embodiments, the artificially-induced modification does not result in any endogenous genes that exhibit an at least about 2, at least about 2.5, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, or at least about 1000 fold change in expression.

In some embodiments, the artificially-introduced modification has minimal impact on local gene expression, for example, as determined by RNA seq or a gene array.

In some embodiments, the artificially-induced modification at a genomic site does not result in any endogenous genes within 300 kb of the modification exhibiting an at least about 2, at least about 2.5, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, or at least about 1000 fold change in expression.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 2 endogenous genes that are within 300 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 3 endogenous genes that are within 300 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 5 endogenous genes that are within 300 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 10 endogenous genes that are within 300 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 15 endogenous genes that are within 300 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 20 endogenous genes that are within 300 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 25 endogenous genes that are within 300 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 50 endogenous genes that are within 300 kb of the genomic site.

In some embodiments, the artificially-induced modification at a genomic site does not result in any genes within 200 kb of the modification exhibiting an at least about 2, at least about 2.5, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, or at least about 1000 fold change in expression.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 2 endogenous genes that are within 200 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 3 endogenous genes that are within 200 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 5 endogenous genes that are within 200 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 10 endogenous genes that are within 200 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 15 endogenous genes that are within 200 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 20 endogenous genes that are within 200 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 25 endogenous genes that are within 200 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 50 endogenous genes that are within 200 kb of the genomic site.

In some embodiments, the artificially-induced modification at a genomic site does not result in any genes within 100 kb of the modification exhibiting an at least about 2, at least about 2.5, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, or at least about 1000 fold change in expression.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 2 endogenous genes that are within 100 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 3 endogenous genes that are within 100 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 5 endogenous genes that are within 100 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 10 endogenous genes that are within 100 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 15 endogenous genes that are within 100 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 20 endogenous genes that are within 100 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 25 endogenous genes that are within 100 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 50 endogenous genes that are within 100 kb of the genomic site.

In some embodiments, the artificially-induced modification at a genomic site does not result in any genes within 50 kb of the modification exhibiting an at least about 2, at least about 2.5, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 350, at least about 400, at least about 450, at least about 500, or at least about 1000 fold change in expression.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 2 endogenous genes that are within 50 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 3 endogenous genes that are within 50 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 5 endogenous genes that are within 50 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 10 endogenous genes that are within 50 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 15 endogenous genes that are within 50 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 20 endogenous genes that are within 50 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 25 endogenous genes that are within 50 kb of the genomic site.

In some embodiments, the artificially-induced modification at the genomic site effects no more than about 0.25, no more than about 0.5, no more than about 1, no more than about 1.5, no more than about 2, no more than about 2.5, no more than about 3, no more than about 4, no more than about 5, no more than about 6, no more than about 7, no more than about 8, no more than about 9, no more than about 10, no more than about 11, no more than about 12, no more than about 13, no more than about 14, no more than about 15, no more than about 20, no more than about 25, no more than about 30, no more than about 35, no more than about 40, no more than about 45, no more than about 50, no more than about 60, no more than about 70, no more than about 80, no more than about 90, no more than about 100, no more than about 150, no more than about 200, no more than about 250, no more than about 300, no more than about 350, no more than about 400, no more than about 450, no more than about 500, or no more than about 1000 fold change in expression of no more than about 50 endogenous genes that are within 50 kb of the genomic site.

In some cases, fold change in expression refers to a fold increase in expression. In some cases, fold change in expression refers to a fold decrease in expression. In some cases, fold change in expression encompasses increases and decreases in expression of at least the recited magnitude.

In some embodiments, the artificially-induced modification at the genomic site does not induce or substantially does not induce expression of any genes that are not expressed in corresponding cells the absence of the artificially-induced modification (for example, are not expressed above a limit of detection). In some embodiments, the artificially-induced modification at the genomic site induces expression of no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 25, no more than 30, no more than 35, no more than 40, no more than 45, no more than 50, no more than 50, no more than 60, no more than 70, no more than 80, no more than 90, no more than 100, no more than 110, no more than 120, no more than 130, no more than 140, no more than 150, no more than 160, no more than 170, no more than 180, no more than 190, no more than 200, no more than 250, no more than 300, no more than 350, no more than 400, no more than 450, no more than 500, no more than 550, no more than 600, no more than 650, no more than 700, or no more than 750 genes that are not expressed in corresponding cells the absence of the artificially-induced modification (for example, are not expressed above a limit of detection).

In some embodiments, the artificially-induced modification at the genomic site does not induce or substantially does not induce expression of any genes within 300 kb of the genomic site that are not expressed in corresponding cells the absence of the artificially-induced modification (for example, are not expressed above a limit of detection). In some embodiments, the artificially-induced modification at the genomic site induces expression of no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 25, no more than 30, no more than 35, no more than 40, no more than 45, no more than 50, no more than 50, no more than 60, no more than 70, no more than 80, no more than 90, no more than 100, no more than 110, no more than 120, no more than 130, no more than 140, no more than 150, no more than 160, no more than 170, no more than 180, no more than 190, no more than 200, no more than 250, no more than 300, no more than 350, no more than 400, no more than 450, no more than 500, no more than 550, no more than 600, no more than 650, no more than 700, or no more than 750 genes within 300 kb of the genomic site that are not expressed in corresponding cells the absence of the artificially-induced modification (for example, are not expressed above a limit of detection).

In some embodiments, the artificially-induced modification at the genomic site does not reduce or substantially does not reduce or abolish expression of any genes that are expressed in corresponding cells the absence of the artificially-induced modification (for example, does not reduce expression from a detectable level to below a limit of detection). In some embodiments, the artificially-induced modification at the genomic site reduces or abolishes expression of no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 25, no more than 30, no more than 35, no more than 40, no more than 45, no more than 50, no more than 50, no more than 60, no more than 70, no more than 80, no more than 90, no more than 100, no more than 110, no more than 120, no more than 130, no more than 140, no more than 150, no more than 160, no more than 170, no more than 180, no more than 190, no more than 200, no more than 250, no more than 300, no more than 350, no more than 400, no more than 450, no more than 500, no more than 550, no more than 600, no more than 650, no more than 700, or no more than 750 genes that are expressed in corresponding cells the absence of the artificially-induced modification (for example, does not reduce expression from a detectable level to below a limit of detection).

In some embodiments, the artificially-induced modification at the genomic site does not reduce or substantially does not reduce or abolish expression of any genes within 300 kb of the genomic site that are expressed in corresponding cells the absence of the artificially-induced modification (for example, does not reduce expression from a detectable level to below a limit of detection). In some embodiments, the artificially-induced modification at the genomic site reduces or abolishes expression of no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 25, no more than 30, no more than 35, no more than 40, no more than 45, no more than 50, no more than 50, no more than 60, no more than 70, no more than 80, no more than 90, no more than 100, no more than 110, no more than 120, no more than 130, no more than 140, no more than 150, no more than 160, no more than 170, no more than 180, no more than 190, no more than 200, no more than 250, no more than 300, no more than 350, no more than 400, no more than 450, no more than 500, no more than 550, no more than 600, no more than 650, no more than 700, or no more than 750 genes within 300 kb of the genomic site that are expressed in corresponding cells the absence of the artificially-induced modification (for example, does not reduce expression from a detectable level to below a limit of detection).

In some embodiments, an artificially-introduced modification can have off target effects that are dependent on the artificially-introduced modification itself, rather than the location in the genome. For example, in some cases, expression of a transgene can impact signaling pathways, kinomic, and/or transcriptomic profiles of a cell expressing the transgene. In some embodiments, such effects can be determined, for example, by comparing transcriptional profiles of cells that express the transgene from multiple integration sites (e.g., other safe harbor sites), and/or cells that are transiently transfected to express the transgene. In some embodiments, such analyses can be used to differentiate between changes in gene expression that are a result of the artificially-introduced modification (e.g., effect of an expressed transgene), and changes in gene expression that are a result of the use of a genomic site (e.g., candidate safe harbor locus) as an integration site. In some embodiments, genes exhibiting changes in expression dependent on transgene expression rather than genomic site can be excluded from counts of genes that are differentially expressed due to the artificially-induced modification at the genomic site.

### D. Artificially-Induced Modifications and Transgenes

In some embodiments, the disclosure provides engineered cells (e.g., populations thereof) comprising artificially-induced modifications in genomic sites disclosed herein, such as safe harbor sites. An artificially-induced modification can comprise an insertion, a deletion, a substitution, or a combination thereof. In some embodiments, an artificially-induced modification can comprise deletion of one or more nucleotides from the genomic site. In some embodiments, an artificially-induced modification can comprise substitution of one or more nucleotides from the genomic site. The artificially-induced modification can comprise an insert sequence, for example, a nucleotide sequence that is not present at the genomic site until the modification is artificially introduced. In some embodiments, an artificially-induced modification can comprise deletion of one or more nucleotides from the genomic site, and an insert sequence. An artificially-induced modification (e.g., an insert sequence) can comprise one or more expression cassettes. An expression cassette can comprise, for example, one or more transgenes operably coupled to one or more regulatory elements, such as promoters. An expression cassette can comprise intervening, non-coding regions as well as regulatory regions and can include 5′ and 3′ ends, transcribed sequences, including 5′ and 3′ untranslated regions (5′-UTR and 3′-UTR), exons and introns, “open reading frame(s)” that encode polypeptide(s), and/or non-transcribed regions including upstream and downstream regulatory regions, enhancers and promoters.

In some embodiments, an artificially-induced modification (e.g., an insert sequence) comprises one expression cassette. In some embodiments, an artificially-induced modification (e.g., an insert sequence) comprises 2, 3, 4, 5, 6, 7, 8, 9, or 10 expression cassettes. In some embodiments, an artificially-induced modification (e.g., an insert sequence) comprises at least 2, at least 3, at least 4, at least 5, at least 6, at least 7, at least 8, at least 9, or at least 10 expression cassettes.

In some embodiments, an artificially-induced modification (e.g., an insert sequence) comprises one transgene. In some embodiments, an artificially-induced modification (e.g., an insert sequence) comprises 2, 3, 4, 5, 6, 7, 8, 9, 10, 11, 12, 13, 14, 15, 16, 17, 18, 19, or 20 transgenes. In some embodiments, an artificially-induced modification (e.g., an insert sequence) comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 transgenes.

In some embodiments, an expression cassette comprises one transgene. In some embodiments, an expression cassette comprises 2, 3, 4, 5, 6, 7, 8, 9, 10, 11, 12, 13, 14, 15, 16, 17, 18, 19, or 20 transgenes. In some embodiments, an expression cassette comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 transgenes.

In cases where an artificially-induced modification (e.g., insert sequence) comprises two or more transgenes, expression of the two or more transgenes can be driven by one promoter, multiple promoters that are the same or different promoters, or a combination thereof.

In cases where an artificially-induced modification (e.g., insert sequence) comprises two or more transgenes, the two or more transgenes can be part of separate transcriptional units, one transcriptional unit (e.g., with separate transgenes separated by cleavable linker(s) or IRES as disclosed herein), or a combination thereof.

In some embodiments, an artificially-induced modification comprises an insert sequence that is at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, at least 2500, at least 2600, at least 2700, at least 2800, at least 2900, at least 3000, at least 3100, at least 3200, at least 3300, at least 3400, at least 3500, at least 3600, at least 3700, at least 3800, at least 3900, at least 4000, at least 4100, at least 4200, at least 4300, at least 4400, at least 4500, at least 4600, at least 4700, at least 4800, at least 4900, at least 5000, at least 5100, at least 5200, at least 5300, at least 5400, at least 5500, at least 5600, at least 5700, at least 5800, at least 5900, at least 6000, at least 6100, at least 6200, at least 6300, at least 6400, at least 6500, at least 6600, at least 6700, at least 6800, at least 6900, at least 7000, at least 7100, at least 7200, at least 7300, at least 7400, at least 7500, at least 7600, at least 7700, at least 7800, at least 7900, at least 8000, at least 8100, at least 8200, at least 8300, at least 8400, at least 8500, at least 8600, at least 8700, at least 8800, at least 8900, at least 9000, at least 9100, at least 9200, at least 9300, at least 9400, at least 9500, at least 9600, at least 9700, at least 9800, at least 9900, at least 10000, at least 2×10\hat{ }4, at least 3×10\hat{ }4, at least 4×10\hat{ }4, at least 5×10\hat{ }4, at least 6×10\hat{ }4, at least 7×10\hat{ }4, at least 8×10\hat{ }4, at least 9×10\hat{ }4, or at least 1×10\hat{ }5 nucleotides in length.

In some embodiments, an artificially-induced modification comprises an insert sequence that is at most 500, at most 550, at most 600, at most 650, at most 700, at most 750, at most 800, at most 850, at most 900, at most 950, at most 1000, at most 1100, at most 1200, at most 1300, at most 1400, at most 1500, at most 1600, at most 1700, at most 1800, at most 1900, at most 2000, at most 2100, at most 2200, at most 2300, at most 2400, at most 2500, at most 2600, at most 2700, at most 2800, at most 2900, at most 3000, at most 3100, at most 3200, at most 3300, at most 3400, at most 3500, at most 3600, at most 3700, at most 3800, at most 3900, at most 4000, at most 4100, at most 4200, at most 4300, at most 4400, at most 4500, at most 4600, at most 4700, at most 4800, at most 4900, at most 5000, at most 5100, at most 5200, at most 5300, at most 5400, at most 5500, at most 5600, at most 5700, at most 5800, at most 5900, at most 6000, at most 6100, at most 6200, at most 6300, at most 6400, at most 6500, at most 6600, at most 6700, at most 6800, at most 6900, at most 7000, at most 7100, at most 7200, at most 7300, at most 7400, at most 7500, at most 7600, at most 7700, at most 7800, at most 7900, at most 8000, at most 8100, at most 8200, at most 8300, at most 8400, at most 8500, at most 8600, at most 8700, at most 8800, at most 8900, at most 9000, at most 9100, at most 9200, at most 9300, at most 9400, at most 9500, at most 9600, at most 9700, at most 9800, at most 9900, at most 10000, at most 2×10\hat{ }4, at most 3×10\hat{ }4, at most 4×10\hat{ }4, at most 5×10\hat{ }4, at most 6×10\hat{ }4, at most 7×10\hat{ }4, at most 8×10\hat{ }4, at most 9×10\hat{ }4, or at most 1×10\hat{ }5 nucleotides in length.

In some embodiments, an artificially-induced modification comprises an insert sequence that is about 50, about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5200, about 5300, about 5400, about 5500, about 5600, about 5700, about 5800, about 5900, about 6000, about 6100, about 6200, about 6300, about 6400, about 6500, about 6600, about 6700, about 6800, about 6900, about 7000, about 7100, about 7200, about 7300, about 7400, about 7500, about 7600, about 7700, about 7800, about 7900, about 8000, about 8100, about 8200, about 8300, about 8400, about 8500, about 8600, about 8700, about 8800, about 8900, about 9000, about 9100, about 9200, about 9300, about 9400, about 9500, about 9600, about 9700, about 9800, about 9900, about 10000, about 2×10\hat{ }4, about 3×10\hat{ }4, about 4×10\hat{ }4, about 5×10\hat{ }4, about 6×10\hat{ }4, about 7×10\hat{ }4, about 8×10\hat{ }4, about 9×10\hat{ }4, or about 1×10≡nucleotides in length.

In some embodiments, an artificially-induced modification at a genomic site of the disclosure includes an expression cassette comprising a transgene. A transgene can encode a cytokine. The cytokine can be secreted. In some embodiments, the cytokine is bound to a cell surface membrane of the engineered cell.

In some embodiments, a transgene encodes 4-1BBL, APRIL, CD153, CD154, CD178, CD70, G-CSF, GITRL, GM-CSF, IFN-α, IFN-β, IFN-γ, IL-1RA, IL-1α, IL-1β, IL-2, IL-3, IL-4, IL-5, IL-6, IL-7, IL-9, IL-10, IL-11, IL-12, IL-13, IL-14, IL-15, IL-16, IL-17, IL-18, IL-20, IL-23, LIF, LIGHT, LT-β, M-CSF, MSP, OSM, OX40L, SCF, TALL-1, TGF-β, TGF-β1, TGF-β2, TGF-β3, TNF-α, TNF-β, TRAIL, TRANCE, or TWEAK. In some embodiments, an engineered cell of the disclosure comprises a transgene that encodes a cytokine and a transgene that encodes a receptor for the cytokine. Such an engineered cell (e.g., engineered NK cell) can exhibit enhanced signaling of the signaling pathway induced by the cytokine and/or the receptor (e.g., induced by the cytokine and/or receptor, such as IL-15/IL-15R).

In some embodiments, a transgene encodes a chemokine. For example, a transgene can encode ACT-2, AMAC-a, ATAC, ATAC, BLC, CCL1, CCL11, CCL13, CCL14, CCL15, CCL16, CCL17, CCL18, CCL19, CCL2, CCL20, CCL21, CCL22, CCL23, CCL24, CCL25, CCL26, CCL27, CCL3, CCL4, CCL5, CCL7, CCL8, CKb-6, CKb-8, CTACK, CX3CL1, CXCL1, CXCL10, CXCL11, CXCL12, CXCL13, CXCL14, CXCL2, CXCL3, CXCL4, CXCL5, CXCL6, CXCL7, CXCL8, CXCL9, DC-CK1, ELC, ENA-78, eotaxin, eotaxin-2, eotaxin-3, Eskine, exodus-1, exodus-2, exodus-3, fractalkine, GCP-2, GROa, GROb, GROg, HCC-1, HCC-2, HCC-4, I-309, IL-8, ILC, IP-10−, I-TAC−, LAG-1, LARC, LCC-1, LD78a, LEC, Lkn-1, LMC, lymphoactin, lymphoactin b, MCAF, MCP-1, MCP-2, MCP-3, MCP-4, MDC, MDNCF, MGSA-a, MGSA-b, MGSA-g, Mig, MIP-1d, MIP-1α, MIP-1β, MIP-2a, MIP-2b, MIP-3, MIP-3α, MIP-3β, MIP-4, MIP-4a, MIP-5, MPIF-1, MPIF-2, NAF, NAP-1, NAP-2, oncostatin, PARC, PF4, PPBP, RANTES, SCM-1a, SCM-1b, SDF-1α/β−, SLC, STCP-1, TARC, TECK, XCL1, or XCL2.

In some embodiments, a transgene encodes a receptor, for example, a respective receptor of a cytokine or chemokine disclosed herein (e.g., an IL-15R). In some embodiments, a transgene encodes a common gamma chain receptor, a common beta chain receptor, an interferon receptor, a TNF family receptor, a TGF-B receptor, Apo3, BCMA, CD114, CD115, CD116, CD117, CD118, CD120, CD120a, CD120b, CD121, CD121a, CD121b, CD122, CD123, CD124, CD126, CD127, CD130, CD131, CD132, CD212, CD213, CD213a1, CD213a13, CD213a2, CD25, CD27, CD30, CD4, CD40, CD95 (Fas), CDw119, CDw121b, CDw125, CDw131, CDw136, CDw137 (41BB), CDw210, CDw217, GITR, HVEM, IL-11R, IL-11Ra, IL-14R, IL-15R, IL-15Ra, IL-18R, IL-18Rα, IL-18Rβ, IL-20R, IL-20Rα, IL-20Rβ, IL-9R, LIFR, LTβR, OPG, OSMR, OX40, RANK, TACI, TGF-βR1, TGF-βR2, TGF-βR3, TRAILR1, TRAILR2, TRAILR3, or TRAILR4. In some embodiments, a transgene encodes CCR1, CCR2, CCR3, CCR4, CCR5, CCR6, CCR7, CCR8, CCR9, CCR10, CX3CR1, CXCR1, CXCR2, CXCR3, CXCR4, CXCR5, XCR1, or XCR1.

In some embodiments, a transgene encodes an NK receptor, for example, an activating NK receptor or an inhibitory NK receptor. In some embodiments, a transgene encodes CD100 (SEMA4D), CD16 (FcgRIIIA), CD160 (BY55), CD244 (2B4, SLAMF4), CD27, CD94− NKG2C, CD94− NKG2E, CD94-NKG2H, CD96, CRTAM, DAP12, DNAM1 (CD226), KIR2DL4, KIR2DS1, KIR2DS2, KIR2DS3, KIR2DS4, KIR2DS5, KIR3DS1, Ly49, NCR, NKG2D (KLRK1, CD314), NKp30 (NCR3), NKp44 (NCR2), NKp46 (NCR1), NKp80 (KLRF1, CLEC5C), NTB-A (SLAMF6), PSGL1, or SLAMF7 (CRACC, CS1, CD319). In some embodiments, a transgene encodes CD161 (NKR-P1A, NK1.1), CD94− NKG2A, CD96, CEACAM1, KIR2DL1, KIR2DL2, KIR2DL3, KIR2DL4, KIR2DL5A, KIR2DL5B, KIR3DL1, KIR3DL2, KIR3DL3, KLRG1, LAIR1, LIR1 (ILT2, LILRB1), Ly49a, Ly49b, NKR-P1A (KLRB1), SIGLEC-10, SIGLEC-11, SIGLEC-14, SIGLEC-16, SIGLEC-3 (CD33), SIGLEC-5 (CD170), SIGLEC-6 (CD327), SIGLEC-7 (CD328), SIGLEC-8, SIGLEC-9 (CD329), SIGLEC-E, SIGLEC-F, SIGLEC-G, SIGLEC-H, or TIGIT.

In some embodiments, a transgene encodes an immune co-receptor, for example, an activating or inhibitory immune co-receptor.

In some embodiments, a transgene encodes 2B4, B7-1, BTLA, CD160, CTLA-4, DR6, Fas, LAG3, LAIR1, Ly108, PD-1, PD-L1, PD1H, TIGIT, TIM1, TIM2, or TIM3. In some embodiments, a transgene encodes 4-1BB, CD2, CD4, CD8, CD21, CD27, CD28, CD30, CD40, CD84, CD226, CD355, CRACC, DcR3, DR3, GITR, HVEM, ICOS, Ly9, Ly108, LIGHT, LTβR, OX40, SLAM, TIM1, or TIM2.

In some embodiments, a transgene encodes a transcription factor, for example, a transcription factor that is active in an immune cell subset, or a transcription factor that directs a stem cell to differentiate into a cell lineage or specific cell type, or that directs an immature immune cell to differentiate into a desired immune cell subset or mature immune cell. Non-limiting examples of transcription factors that can be encoded by a transgene of the disclosure include AP-1, Bcl6, E2A, EBF, Eomes, FoxP3, GATA3, Id2, Ikaros, IRF, IRF1, IRF2, IRF3, IRF3, IRF7, NFAT, NFkB, Pax5, PLZF, PU.1, ROR-gamma-T, STAT, STAT1, STAT2, STAT3, STAT4, STAT5, STAT5A, STAT5B, STAT6, T-bet, TCF7, and ThPOK.

In some embodiments, a transgene encodes CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD71, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), or CD326 (EpCAM).

A transgene can encode a CD16 variant, for example, for enhanced CD16 signaling as compared to a control cell. A transgene can encode CD137, CD80, CD86, or DAP10 (e.g., with or without point mutation). A transgene can encode CD3, CD4, CD80, 41BBL, or CD131.

A transgene can encode a chimeric polypeptide receptor, for example, an antigen-recognition receptor comprising an antigen binding moiety capable of binding to an antigen, as provided in the present disclosure. In some examples, an engineered cell can comprise a plurality of different chimeric polypeptide receptors to specifically bind a plurality of different antigens, one or more of which can be inserted into a genetic site (e.g., safe harbor locus) of the disclosure. In some examples, the chimeric polypeptide receptor can comprise comprises a plurality of antigen binding moieties to specifically bind a plurality of different antigens.

A chimeric polypeptide receptor can comprises a T cell receptor fusion protein (TFP). The term “T cell receptor fusion protein” or “TFP” generally refers to a recombinant polypeptide construct comprising (i) one or more antigen binding moieties (e.g., monospecific or multispecific), (ii) at least a portion of TCR extracellular domain, (iii) at least a portion of TCR transmembrane domain, and (iv) at least a portion of TCR intracellular domain.

A chimeric polypeptide receptor can comprises a chimeric antigen receptor (CAR). The term “chimeric antigen receptor” or “CAR” generally refers to a recombinant polypeptide construct comprising at least an extracellular antigen binding moiety (e.g., an antigen binding domain), a transmembrane domain, and a cytoplasmic signaling domain (also referred to herein as a “signaling domain” an “intracellular signaling domain” or an “intrinsic signaling domain”) comprising a functional signaling domain derived from a stimulatory molecule. In some cases, the stimulatory molecule may be the zeta chain associated with the T cell receptor complex. In some cases, the intracellular signaling domain further comprises one or more costimulatory domains, for example, a functional signaling domain derived from at least one costimulatory molecule or receptor. In some cases, the costimulatory molecule may comprise 4-1BB (i.e., CD137), CD27, and/or CD28. In one aspect, the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane.

A CAR may be a first-, second-, third-, or fourth-generation CAR system, a functional variant thereof, or any combination thereof. First-generation CARs (e.g., CD19R or CD19CAR) include an antigen binding domain with specificity for a particular antigen (e.g., an antibody or antigen-binding fragment thereof such as an scFv, a Fab fragment, a VHH domain, or a VH domain of a heavy-chain only antibody), a transmembrane domain derived from an adaptive immune receptor (e.g., the transmembrane domain from the CD28 receptor), and a signaling domain derived from an adaptive immune receptor (e.g., one or more (e.g., three) ITAM domains derived from the intracellular region of the CD3 (receptor or FcεRIγ). Second-generation CARs modify the first-generation CAR by addition of a costimulatory domain to the intracellular signaling domain portion of the CAR (e.g., derived from costimulatory receptors that act alongside T-cell receptors such as CD28, CD137/4-1BB, and CD134/OX40), which abrogates the need for administration of a co-factor (e.g., IL-2) alongside a first-generation CAR. Third-generation CARs add multiple costimulatory domains to the intracellular signaling domain portion of the CAR (e.g., CD3ζ-CD28-OX40, or CD3ζ-CD28-41BB). Fourth-generation CARs modify second- or third-generation CARs by the addition of an activating cytokine (e.g., IL-12, IL-23, or IL-27) to the intracellular signaling portion of the CAR (e.g., a signaling domain from a receptor of the activating cytokine between one or more of the costimulatory domains and the CD3(ITAM domain) or under the control of a CAR-induced promoter (e.g., the NFAT/IL-2 minimal promoter). In some cases, a CAR may be a new generation CAR system that is different than the first-, second-, third-, or fourth-generation CAR system as disclosed herein.

A hinge domain (e.g., the linker between the extracellular antigen binding domain and the transmembrane domain) of a CAR in the engineered immune cell (e.g., engineered NK cell) as disclosed herein can comprise a full length or at least a portion of the native or modified transmembrane region of CD3D, CD3E, CD3G, CD3c CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor polypeptide.

A transmembrane domain of a CAR in the engineered immune cell (e.g., engineered NK cell) as disclosed herein can comprise a full length or at least a portion of the native or modified transmembrane region of CD3D, CD3E, CD3G, CD3c CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor polypeptide.

The hinge domain and the transmembrane domain of a CAR as disclosed herein (e.g., for the engineered immune cell, such as the engineered NK cell) can be derived from the same protein (e.g., CD8). Alternatively, the hinge domain and the transmembrane domain of the CAR as disclosed herein can be derived from different proteins.

A signaling domain of a CAR can comprise at least or up to about 1 signaling domain, at least or up to about 2 signaling domains, at least or up to about 3 signaling domains, at least or up to about 4 signaling domains, at least or up to about 5 signaling domains, at least or up to about 6 signaling domains, at least or up to about 7 signaling domains, at least or up to about 8 signaling domains, at least or up to about 9 signaling domains, or at least or up to about 10 signaling domains.

A signaling domain (e.g., an intracellular signaling domain, a costimulatory domain, and/or a signaling peptide of the intracellular signaling domain) of a CAR in the engineered immune cell (e.g., engineered NK cell) as disclosed herein can comprise a full length or at least a portion of a polypeptide of CD3ζ, 2B4, DAP10, DAP12, DNAM1, CD137 (41BB), IL21, IL7, IL12, IL15, NKp30, NKp44, NKp46, NKG2C, NKG2D, or any combination thereof.

Alternatively or in addition, the CAR signaling domain (e.g., intracellular signaling domain or costimulatory domain) can comprise a full length or at least a portion of a polypeptide of CD27, CD28, 4-1BB, OX40, ICOS, PD-1, LAG-3, 2B4, BTLA, DAP10, DAP12, CTLA-4, or NKG2D, or any combination thereof.

In some embodiments, the CAR comprises at least a CD8 transmembrane domain and one or more of: (i) 2B4 signaling domain and (ii) DAP10 signaling domain.

In some embodiments, the chimeric polypeptide receptor (e.g., TFP or CAR) comprises at least (i) CD8 transmembrane domain, (ii) 2B4 signaling domain, and (iii) DAP10 signaling domain. The 2B4 signaling domain can be flanked by the CD8 transmembrane domain and the DAP10 signaling domain. Alternatively, the DAP10 signaling domain can be flanked by the CD8 transmembrane domain and the 2B4 signaling domain. In some cases, the chimeric polypeptide receptor as disclosed herein can further comprise yet an additional signaling domain derived from CD3ζ.

An antigen (i.e., a target antigen) of an antigen binding moiety of a chimeric polypeptide receptor (e.g., TFP or CAR) as disclosed herein can be a cell surface marker, a secreted marker, or an intracellular marker.

Non-limiting examples of an antigen (i.e., a target antigen) of an antigen binding moiety of a chimeric polypeptide receptor (e.g., TFP or CAR) as disclosed herein can include ADGRE2, carbonic anhydrase IX (CA1X), CCRI, CCR4, carcinoembryonic antigen (CEA), CD3ζ, CD5, CD8, CD10, CD19, CD20, CD22, CD30, CD33, CD34, CD38, CD41, CD44, CD44V6, CD49f, CD56, CD70, CD74, CD99, CD133, CD138, CD269 (BCMA), CD S, CLEC12A, an antigen of a cytomegalovirus (CMV) infected cell (e.g., a cell surface antigen), epithelial glycoprotein2 (EGP 2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-a, Ganglioside G2 (GD2), Ganglioside G3 (GD3), gp100, human Epidermal Growth Factor Receptor 2 (HER-2), human telomerase reverse transcriptase (hTERT), ICAM-1, Integrin B7, Interleukin-13 receptor subunit alpha-2 (IL-13Ra2), x-light chain, kinase insert domain receptor (KDR), Kappa, Lewis A (CA19.9), Lewis Y (LeY), L1 cell adhesion molecule (L1-CAM), LILRB2, MART-1, melanoma antigen family A 1 (MAGE-A1), MICA/B, Mucin 1 (Muc-1), Mucin 16 (Muc-16), Mesothelin (MSLN), NKCSI, NKG2D ligand, c-Met, cancer-testis antigen NY-ESO-1, NY-ESO-2, oncofetal antigen (h5T4), PRAIVIE, prostate stem cell antigen (PSCA), PRAME prostate-specific membrane antigen (PSMA), ROR1, tumor-associated glycoprotein 72 (TAG-72), TIM-3, TRBC1, TRBC2, vascular endothelial growth factor R2 (VEGF-R2), Wilms tumor protein (WT-1), and various pathogen antigens (e.g., pathogen antigens derived from a virus, bacteria, fungi, parasite or protozoa capable of causing disease). In some examples, a pathogen antigen is derived from HIV, HBV, EBV, HPV, Lasse Virus, Influenza Virus, or Coronavirus.

Additional examples of the antigen (i.e., a target antigen) of the antigen binding moiety of the chimeric polypeptide receptor as disclosed herein can include 1-40-β-amyloid, 4-1BB, 5AC, 5T4, activin receptor-like kinase 1, ACVR2B, adenocarcinoma antigen, AGS-22M6, alpha-fetoprotein, angiopoietin 2, angiopoietin 3, anthrax toxin, AOC3 (VAP-1), B7-H3, Bacillus anthracis anthrax, BAFF, beta-amyloid, B-lymphoma cell, C242 antigen, C5, CA-125, Canis lupus familiaris IL31, carbonic anhydrase 9 (CA-IX), cardiac myosin, CCL11 (eotaxin-1), CCR4, CCR5, CD11, CD18, CD125, CD140a, CD147 (basigin), CD15, CD152, CD154 (CD40L), CD19, CD2, CD20, CD200, CD22, CD221, CD25 (a chain of IL-2receptor), CD27, CD274, CD28, CD3, CD3 epsilon, CD30, CD33, CD37, CD38, CD4, CD40, CD40 ligand, CD41, CD44 v6, CD5, CD51, CD52, CD56, CD6, CD70, CD74, CD79B, CD80, CEA, CEA-related antigen, CFD, ch4D5, CLDN18.2, Clostridium difficile, clumping factor A, CSF1R, CSF2, CTLA-4, C—X—C chemokine receptor type 4, cytomegalovirus, cytomegalovirus glycoprotein B, dabigatran, DLL4, DPP4, DR5, E. coli shiga toxin type-1, E. coli shiga toxin type-2, EGFL7, EGFR, endotoxin, EpCAM, episialin, ERBB3, Escherichia coli, F protein of respiratory syncytial virus, FAP, fibrin II beta chain, fibronectin extra domain-B, folate hydrolase, folate receptor 1, folate receptor alpha, Frizzled receptor, ganglioside GD2, GD2, GD3 ganglioside, glypican 3, GMCSF receptor α-chain, GPNMB, growth differentiation factor 8, GUCY2C, hemagglutinin, hepatitis B surface antigen, hepatitis B virus, HER1, HER2/neu, HER3, HGF, HHGFR, histone complex, HIV-1, HLA-DR, HNGF, Hsp90, human scatter factor receptor kinase, human TNF, human beta-amyloid, ICAM-1 (CD54), IFN-α, IFN-γ, IgE, IgE Fc region, IGF-1 receptor, IGF-1, IGHE, IL17A, IL17F, IL20, IL-12, IL-13, IL-17, IL-1β, IL-22, IL-23, IL-31RA, IL-4, IL-5, IL-6, IL-6 receptor, IL-9, ILGF2, influenza A hemagglutinin, influenza A virus hemagglutinin, insulin-like growth factor I receptor, integrin α4β7, integrin α4, integrin α5β1, integrin α7 β7, integrin αIIbβ3, integrin αvβ3, interferon α/β receptor, interferon gamma-induced protein, ITGA2, ITGB2 (CD18), KIR2D, Lewis-Y antigen, LFA-1 (CD11a), LINGO-1, lipoteichoic acid, LOXL2, L-selectin (CD62L), LTA, MCP-1, mesothelin, MIF, MS4A1, MSLN, MUC1, mucin CanAg, myelin-associated glycoprotein, myostatin, NCA-90 (granulocyte antigen), neural apoptosis-regulated proteinase 1, NGF, N-glycolylneuraminic acid, NOGO-A, Notch receptor, NRP1, Oryctolagus cuniculus, OX-40, oxLDL, PCSK9, PD-1, PDCD1, PDGF-Rα, phosphate-sodium co-transporter, phosphatidylserine, platelet-derived growth factor receptor beta, prostatic carcinoma cells, Pseudomonas aeruginosa, rabies virus glycoprotein, RANKL, respiratory syncytial virus, RHD, Rhesus factor, RON, RTN4, sclerostin, SDC1, selectin P, SLAMF7, SOST, sphingosine-1-phosphate, Staphylococcus aureus, STEAP1, TAG-72, T-cell receptor, TEM1, tenascin C, TFPI, TGF-β 1, TGF-β 2, TGF-β, TNF-α, TRAIL-R1, TRAIL-R2, tumor antigen CTAA16.88, tumor specific glycosylation of MUC1, tumor-associated calcium signal transducer 2, TWEAK receptor, TYRP1(glycoprotein 75), VEGFA, VEGFR1, VEGFR2, vimentin, and VWF.

Additional examples of the antigen (i.e., a target antigen) of the antigen binding moiety of the chimeric polypeptide receptor as disclosed herein can include 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain.

An antigen binding moiety of the chimeric polypeptide receptor as disclosed herein can include an antibody, a fragment thereof, or a variant thereof. Such antibody can be a natural antibody (e.g., naturally secreted by a subject's immune cell, such as B cells), a synthetic antibody, or a modified antibody. In some cases, the antigen binding moiety of the chimeric polypeptide receptor as disclosed herein can include an antigen-binding fragment of an antibody from the group comprising 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, zlintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatumumab, concizumab, Cotara (iodine I-131 derlotuximab biotin), cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hu14.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imeiromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L191L2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDIO680, MEDI-3617, MEDI-551 (inebilizumab), MEDI-565, MEDI6469, mepolizumab, metelimumab, MGB453, MGD006/S80880, MGD007, MGD009, MGDO11, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, RO7009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIV1A, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRBS07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, and zolimomab aritox.

In some embodiments, an antigen binding moiety of the chimeric polypeptide receptor as disclosed herein binds to an antibody, a fragment thereof, or a variant thereof. Such antibody can be a natural antibody (e.g., naturally secreted by a subject's immune cell, such as B cells), a synthetic antibody, or a modified antibody. In some cases, the antigen binding moiety of the chimeric polypeptide receptor as disclosed herein can bind to an antibody (e.g., a constant domain or Fc domain thereof) from the group comprising 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, zlintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatumumab, concizumab, Cotara (iodine I-131 derlotuximab biotin), cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hul4.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imeiromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L19IL2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDIO680, MEDI-3617, MEDI-551 (inebilizumab), MEDI-565, MEDI6469, mepolizumab, metelimumab, MGB453, MGD006/S80880, MGD007, MGD009, MGDO11, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, RO7009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIV1A, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRBS07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, and zolimomab aritox.

In some embodiments, the chimeric polypeptide receptor (e.g., TFP or CAR) comprises an antigen binding domain, and the antigen binding domain is capable of binding specifically and preferentially to an antigen comprising one or more members selected from the group comprising BCMA, CD20, CD22, CD30, CD33, CD38, CD70, Kappa, Lewis Y, NKG2D ligand, ROR1, NY-ESO-1, NY-ESO-2, MART-1, and gp100. Non-limiting examples of the NKG2D ligand comprises one or more members selected from the group comprising of MICA, MICB, ULBP1, ULBP2, ULBP3, ULBP4, ULBP5, and ULBP6.

In some embodiments, the chimeric polypeptide receptor (e.g., TFP or CAR) comprises an antigen binding domain, and the antigen binding domain is capable of binding specifically and preferentially to CD38.

In some embodiments, an expression cassette, an artificially-induced modification, or an engineered cell comprises at least 1, at least 2, at least 3, at least 4, at least 5, or more different types of chimeric polypeptide receptors.

A transgene can encode a safety switch. In some embodiments, a transgene encodes a safety switch capable of effecting death of the engineered cell. In some embodiments, a safety switch can activate a prodrug to elicit killing of the engineered cell. In some cases, the safety switch can comprise one or more members selected from the group consisting of caspase (e.g., caspase 3, 7, or 9), thymidine kinase, cytosine deaminase, modified EGFR, B-cell CD20, and functional variants thereof. In some cases, the safety switch can be activated via an activator (e.g., a small molecule or a protein, such as an antibody) for post-translational, temporal, and/or site-specific regulation of death (or depletion) of the subject engineered cell. Non-limiting examples of a safety switch and its activator can include Caspase 9 (or caspase 3 or 7) and AP1903; thymidine kinase (TK) and ganciclovir (GCV); and cytosine deaminase (CD) and 5-fluorocytosine (5-FC). Alternatively or in addition, modified epidermal growth factor receptor (EGFR) containing epitope recognized by an antibody (e.g., anti-EGFR Ab, such as cetuximab) can be used to deplete the engineered cells when the subject cells are exposed to the antibody.

A transgene can encode an immune regulator polypeptide, for example, one or more members selected from the group consisting of HLA-E, CD47, CD113, PDL1, PDL2, A2AR, HLA-G, TGF-beta, CCL21, IL10, CD46, CD55, and CD59.

A transgene can encode an antibody, a fragment thereof (e.g., an antigen-binding fragment thereof), or a variant thereof. In some cases, a transgene encodes an antibody or antigen-binding fragment of 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, zlintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatumumab, concizumab, Cotara (iodine I-131 derlotuximab biotin), cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hu14.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imeiromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L191L2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDIO680, MEDI-3617, MEDI-551 (inebilizumab), MEDI-565, MEDI6469, mepolizumab, metelimumab, MGB453, MGD006/S80880, MGD007, MGD009, MGDO11, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, RO7009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIV1A, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRBS07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, zolimomab aritox, a derivative thereof, or a combination thereof (for example, a multi-specific antibody, such as a bispecific antibody).

In some embodiments, a transgene encodes an antibody that specifically binds to a cell surface protein is an antigen expressed by a cancerous cell. In some embodiments, a transgene encodes an antibody that specifically binds to a neoepitope. In some embodiments, a transgene encodes an antibody that specifically binds to a tumor associated antigen. In some embodiments, a transgene encodes an antibody that specifically binds to alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAP1B, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Ra, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, anti-transferrin receptor, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, or transient receptor potential cation channel, subfamily M, member 4).

In some embodiments, a transgene encodes an immune checkpoint modulator, for example, an immune checkpoint inhibitor. An immune checkpoint inhibitor can be an antibody or antigen-binding fragment thereof that binds to and inhibits the activity of an immune checkpoint molecule, for example, to reduce the inhibitory effect of the immune checkpoint molecule on the immune response, thereby promoting an immune response, such as an anti-cancer immune response.

In some embodiments, a transgene encodes a fusion protein. In some embodiments, a transgene encodes an Fc fusion protein. In some embodiments, a transgene encodes a receptor-based biologic, for example, a protein that comprises domains from one or more VEGF receptors or one or more TNF receptors, e.g., in an Fc fusion.

In some embodiments, a transgene encodes a bone morphogenetic protein, an enzyme, a growth factor, a hormone, a kinase, a phosphatase, or a thrombolytic. In some embodiments, a transgene encodes insulin.

In some embodiments, a transgene encodes a reporter gene, for example, a fluorescent or luminescent protein.

In some embodiments, a transgene encodes an RNA that is not translated into a protein. In some embodiments, a transgene encodes an antisense oligoribonucleotide, a siRNA, a tRNA, an rRNA, a snRNA, a shRNA, microRNA, or a non-coding RNA.

In some embodiments, a transgene encodes a gene editing system component, for example, a nuclease disclosed herein. Integration of a transgene encoding a gene editing system component disclosed herein can facilitate subsequent gene editing of a cell, for example, by requiring less components to be delivered to the cell to effect gene editing, for example, a gRNA and repair template, but not the nuclease.

An expression cassette or transgene of the disclosure can encode a linker that joins to domains of a polypeptide. In some instances, the linker is a rigid linker. In other instances, the linker is a flexible linker. In some cases, the linker is a non-cleavable linker. In other cases, the linker is a cleavable linker. In additional cases, the linker comprises a linear structure, or a non-linear structure (e.g., a cyclic structure).

An expression cassette or transgene of the disclosure can encode a cleavable linker. A cleavable linker as disclosed herein can comprise a self-cleaving peptide, such as a self-cleaving 2A peptide. Self-cleaving peptides can be found in members of the Picornaviridae virus family, including aphthoviruses such as foot-and-mouth disease virus (FMDV), equine rhinitis A virus (ERAV), Thosea asigna virus (TaV) and porcine tescho virus-1 (PTV-I), and cardioviruses such as Theilovirus (e.g., Theiler's murine encephalomyelitis) and encephalomyocarditis viruses. Non-limiting examples of the self-cleaving 2A peptide can include “F2A”, “E2A”, “P2A”, “T2A”, and functional variants thereof. In some embodiments, the linker is a pH-sensitive linker. In one instance, the linker is cleaved under basic pH conditions. In other instance, the linker is cleaved under acidic pH conditions. In some embodiments, the linker is cleaved in vivo by endogenous enzymes (e.g., proteases) such as serine proteases including but not limited to thrombin, metalloproteases, furin, cathepsin B, necrotic enzymes (e.g., calpains), and the like.

An expression cassette or a transgene can comprise one or more internal ribosome entry site(s) (IRES).

In some embodiments, an engineered cell of the disclosure further comprises one or more artificially-induced modifications outside of certain genomic sites (e.g., safe harbor loci) of the disclosure.

In some embodiments, an engineered cell of the disclosure comprises an artificially-induced modification that reduces expression or activity of PD1, CTLA-4, TIM-3, KIR2D, CD94, NKG2A, NKG2D, TIGIT, CD96, LAG3, TIGIT, TGF beta receptor, 2B4, SHIP2, or a combination thereof.

In some embodiments, an engineered cell of the disclosure comprises an artificially-induced modification that reduces expression or activity of B2M, CIITA, TAP1, TAP2, tapasin, NLRC5, RFXANK, RFX5, RFXAP, CD80, CD86, ICOSL, CD40L, ICAM1, MICA, MICB, ULBP1, HLA-E, CD47, CD113, PDL1, PDL2, A2AR, HLA-G, TGF-beta, CCL21, IL10, CD46, CD55, CD59, or a combination thereof.

In some embodiments, an engineered cell of the disclosure comprises an artificially-induced modification that reduces expression or activity of CD38.

In some cases, an endogenous T cell receptor (TCR) of the engineered cell of the present disclosure can be inactivated. In some examples, a function of the endogenous TCR of the engineered cell can be inhibited by an inhibitor. In some examples, a gene encoding a subunit of the endogenous TCR can be inactivated (e.g., edited via action of the gene editing moiety as disclosed herein) such that the endogenous TCR is inactivated. The gene encoding the subunit of endogenous TCR can be one or more of: TCRα, TCRβ, CD3R, CD36, CD37, and CD3ζ.

A transgene can be operably coupled to one or more regulatory elements, such as promoters. A promoter can be, for example, a constitutive, inducible, temporal, tissue-specific, and/or cell type-specific promoter. A promoter can be a promoter that is active in the engineered cell, for example, active in and/or specific to any cell type disclosed herein. A promoter can be an endogenous human promoter. A promoter can be a modified human promoter. A promoter can be an artificial promoter. In some embodiments, a promoter can be an endogenous promoter, for example, the same promoter that drives expression of a transgene in an organism. In some embodiments, a promoter can be a heterologous promoter, for example, a promoter that is different than a promoter that is operatively coupled to the transgene or a wild type version of the transgene in an organism. A promoter can be a viral promoter.

Non-limiting examples of promoters that can be used include hEF-1a, CMV, EF1a, PGK, CAG, and UBC. Non-limiting examples of constitutive promoters include human β-actin (ACTB), cytomegalovirus (CMV), elongation factor-1α, (EF1α), phosphoglycerate kinase (PGK) ubiquitinC (UbC), SV40, and CAGC promoters. Non-limiting examples of inducible promoters include chemically-inducible promoters (e.g., TET-ON and TET-OFF) and temperature-inducible promoters.

In some embodiments, a promoter that can be used is responsive to an immune system transcription factor, such as an AP-1, Bcl6, E2A, EBF, Eomes, FoxP3, GATA3, Id2, Ikaros, IRF, IRF1, IRF2, IRF3, IRF3, IRF7, NFAT, NFkB, Pax5, PLZF, PU.1, ROR-gamma-T, STAT, STAT1, STAT2, STAT3, STAT4, STAT5, STAT5A, STAT5B, STAT6, T-bet, TCF7, or ThPOK transcription factor.

In some embodiments, a promoter that can be used is responsive to an NK cell transcription factor, for example, Aiolos, E4bp4, Eomes, Ets1, FoxO1, Gata2, Gata3, Helios, id2, Ikaros, IRF2, Nfil3, Notch, PU.1, Runx3, T-bet, Tox1/2, or Tox2.

In some embodiments, a promoter that can be used is responsive to an embryonic stem cell transcription factor, such as Brachyury, EOMES, FoxC2, FoxD3, FoxF1, FoxH1, FoxO1/FKHR, GATA-2, GATA-3, GBX2, Goosecoid, HES-1, HNF-3 alpha/FoxA1, c-Jun, KLF2, KLF4, KLF5, c-Maf, Max, MEF2C, MIXL1, MTF2, c-Myc, Nanog, NFkB/IkB Activators, NFkB/IkB Inhibitors, NFkB1, NFkB2, Oct-3/4, Otx2, p53, Pax2, Pax6, PRDM14, Rex-1/ZFP42, SALL1, SALL4, Smad1, Smad2, Smad2/3, Smad3, Smad4, Smad5, Smad8, Snail, SOX2, SOX7, SOX15, SOX17, STAT Activators, STAT Inhibitors, STAT3, SUZ12, TBX6, TCF-3/E2A, THAP11, UTF1, WDR5, WT1, ZNF206, or ZNF281.

In some embodiments, a promoter that can be used is responsive to an iPSC transcription factor, such as KLF2, KLF4, c-Maf, c-Myc, Nanog, Oct-3/4, p53, SOX1, SOX2, SOX3, SOX15, SOX18, or TBX18.

In some embodiments, a promoter that can be used is responsive to a hematopoietic stem cell transcription factor, such as AHR, Aiolos/IKZF3, CDX4, CREB, DNMT3A, DNMT3B, EGR1, Fox03, GATA-1, GATA-2, GATA-3, Helios, HES-1, HHEX, HIF-1 alpha/HIF1A, HMGB1/HMG-1, HMGB3, Ikaros, c-Jun, LMO2, LMO4, c-Maf, MafB, MEF2C, MYB, c-Myc, NFATC2, NFIL3/E4BP4, Nrf2, p53, PITX2, PRDM16/MEL1, Prox1, PU.1/Spi-1, RUNX1/CBFA2, SALL4, SCL/Tal1, Smad2, Smad2/3, Smad4, Smad7, Spi-B, STAT Activators, STAT Inhibitors, STAT3, STAT4, STAT5a, STAT6, or TSC22.

In some embodiments, a promoter that can be used is responsive to an epithelial stem cell transcription factor, such as ASCL2/Mash2, CDX2, DNMT1, ELF3, Ets-1, FoxM1, FoxN1, GATA-6, Hairless, HNF-4 alpha/NR2A1, IRF6, c-Maf, MITF, Miz-1/ZBTB17, MSX1, MSX2, MYB, c-Myc, Neurogenin-3, NFATC1, NKX3.1, Nrf2, p53, p63/TP73L, Pax2, Pax3, RUNX1/CBFA2, RUNX2/CBFA1, RUNX3/CBFA3, Smad1, Smad2, Smad2/3, Smad4, Smad5, Smad7, Smad8, Snail, SOX2, SOX9, STAT Activators, STAT Inhibitors, STAT3, SUZ12, TCF-3/E2A, or TCF7/TCF1.

In some embodiments, a promoter that can be used is responsive to a mesenchymal stem cell transcription factor, such as DUX4, DUX4/DUX4c, DUX4c, EBF-1, EBF-2, EBF-3, ETV5, FoxC2, FoxF1, GATA-4, GATA-6, HMGA2, c-Jun, MYF-5, Myocardin, MyoD, Myogenin, NFATC2, p53, Pax3, PDX-1/IPF1, PLZF, PRDM16/MEL1, RUNX2/CBFA1, Smad1, Smad3, Smad4, Smad5, Smad8, Smad9, Snail, SOX2, SOX9, SOX11, STAT Activators, STAT Inhibitors, STAT1, STAT3, TBX18, Twist-1, or Twist-2.

In some embodiments, a promoter that can be used is responsive to cancer stem cell transcription factor, such as Androgen R/NR3C4, AP-2 gamma, beta-Catenin, beta-Catenin Inhibitors, Brachyury, CREB, ER alpha/NR3A1, ER beta/NR3A2, FoxM1, Fox03, FRA-1, GLI-1, GLI-2, GLI-3, HIF-1 alpha/HIF1A, HIF-2 alpha/EPAS1, HMGA1B, c-Jun, JunB, KLF4, c-Maf, MCM2, MCM7, MITF, c-Myc, Nanog, NFkB/IkB Activators, NFkB/IkB Inhibitors, NFkB1, NKX3.1, Oct-3/4, p53, PRDM14, Snail, SOX2, SOX9, STAT Activators, STAT Inhibitors, STAT3, TAZ/WWTR1, TBX3, Twist-1, Twist-2, WT1, or ZEB1.

In some embodiments, a promoter that can be used is responsive to a cancer-related transcription factor, such as ASCL1/Mash1, ASCL2/Mash2, ATF1, ATF2, ATF4, BLIMP1/PRDM1, CDX2, CDX4, DLX5, DNMT1, E2F-1, EGR1, ELF3, Ets-1, FosB/G0S3, FoxC1, FoxC2, FoxF1, GADD153, GATA-2, HMGA2, HMGB1/HMG-1, HNF-3 alpha/FoxA1, HNF-6/ONECUT1, HSF1, ID1, ID2, JunD, KLF10, KLF12, KLF17, LMO2, MEF2C, MYCL1/L-Myc, NFkB2, Oct-1, p63/TP73L, Pax3, PITX2, Prox1, RAP80, Rex-1/ZFP42, RUNX1/CBFA2, RUNX3/CBFA3, SALL4, SCL/Tal1, Sirtuin 2/SIRT2, Smad3, Smad4, Smad5, SOX11, STAT5a/b, STAT5a, STAT5b, TCF7/TCF1, TORC1, TORC2, TRIM32, TRPS1, or TSC22.

### E. Cell Types

Any one of the populations of engineered cells disclosed herein can comprise cells of any suitable cell type or lineage disclosed herein. The engineered cells disclosed herein can be engineered ex vivo, in vitro, and in some cases, in vivo.

Non limiting examples of cell types that can be engineered cells of the disclosure include a lymphoid cell, such as a B cell, a T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell, Reticulocyte, Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver cells (e.g., Hepatocyte, or Kupffer cell), Cartilage/bone/muscle; bone cells, including Osteoblast, Osteocyte, Osteoclast, teeth cells, (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte, Epidermal basal cell, Keratinocyte of fingernails and toenails, Nail bed basal cell, Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell, Wet stratified barrier epithelial cells, Surface epithelial cell of stratified squamous epithelium of cornea, tongue, oral cavity, esophagus, anal canal, distal urethra and vagina, basal cell of epithelia of cornea, tongue, oral cavity, esophagus, anal canal, distal urethra and vagina, Urinary epithelium cell, Exocrine secretory epithelial cells, Salivary gland mucous cell, Salivary gland serous cell, Von Ebner's gland cell in tongue, Mammary gland cell, Lacrimal gland cell, Ceruminous gland cell in ear, Eccrine sweat gland dark cell, Eccrine sweat gland clear cell. Apocrine sweat gland cell, Gland of Moll cell in eyelid, Sebaceous gland cell, Bowman's gland cell in nose, Brunner's gland cell in duodenum, Seminal vesicle cell, Prostate gland cell, Bulbourethral gland cell, Bartholin's gland cell, Gland of Littre cell, Uterus endometrium cell, Isolated goblet cell of respiratory and digestive tracts, Stomach lining mucous cell, Gastric gland zymogenic cell, Gastric gland oxyntic cell, Pancreatic acinar cell, Paneth cell of small intestine, Type II pneumocyte of lung, Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell, Macula densa cell of kidney, Metabolism and storage cells, Barrier function cells (e.g., Lung, Gut, Exocrine Glands and Urogenital Tract), Kidney, Type I pneumocyte, Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte, Megakaryocyte, Monocyte, Connective tissue macrophage (various types), Epidermal Langerhans cell, Osteoclast, Dendritic cell, Microglial cell, Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, neurons, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell, Spermatozoon, Nurse cells, Ovarian follicle cell, Sertoli cell, Thymus epithelial cell, Interstitial cells, Interstitial kidney cells, common myeloid progenitors, common lymphoid progenitors, and stem cells that are differentiated into or are to be differentiated into any cell type disclosed herein.

Any one of the populations of engineered cells disclosed herein can be a population of engineered immune cells.

An population of engineered cells can comprise, for example, lymphocytes, T cells, CD4+ T cells, CD8+ T cells, alpha-beta T cells, gamma-delta T cells, T regulatory cells (Tregs), cytotoxic T lymphocytes, Th1 cells, Th2 cells, Th17 cells, Th9 cells, naïve T cells, memory T cells, effector T cells, effector-memory T cells (TEM), central memory T cells (TCM), resident memory T cells (TRM), follicular helper T cells (TFH), Natural killer T cells (NKTs), tumor-infiltrating lymphocytes (TILs), Natural killer cells (NKs), Innate Lymphoid Cells (ILCs), ILC1 cells, ILC2 cells, ILC3 cells, lymphoid tissue inducer (LTi) cells, B cells, B1 cells, B1a cells, B1b cells, B2 cells, plasma cells, B regulatory cells, memory B cells, marginal zone B cells, follicular B cells, germinal center B cells, antigen presenting cells (APCs), monocytes, macrophages, M1 macrophages, M2 macrophages, tissue-associated macrophages, dendritic cells, plasmacytoid dendritic cells, neutrophils, mast cells, basophils, eosinophils, common myeloid progenitors, common lymphoid progenitors, or any combination thereof. In some embodiments, a population of engineered cells comprises NK cells. In some embodiments, a population of engineered cells is a population of NK cells. In some embodiments, a population of engineered cells comprises T cells. In some embodiments, a population of engineered cells is a population of T cells.

In some embodiments, an engineered immune cell can induce an immune response towards a target cell. The target cell can be, for example, a diseased cell, a cancer cell, a tumor cell, etc.

Immune cells can be engineered to exhibit enhanced half-life as compared to control cells (e.g., non-engineered immune cells). Immune cells can be engineered to exhibit enhanced proliferation as compared to control cells. Immune cells can be engineered to effectively and specifically target diseased cells (e.g., cancer cells) that a control cell otherwise is insufficient or unable to target.

Conditions appropriate for T cell culture can include an appropriate media (e.g., Minimal Essential Media or RPMI Media 1640, TexMACS (Miltenyi) or, X-vivo 5, (Lonza)) that may contain factors necessary for proliferation and viability, including serum. In some cases, serum-free medium is used. In an aspect, cells can be maintained under conditions necessary to support growth; for example, an appropriate temperature (e.g., 37° C.) and atmosphere (e.g., air plus 5% C02). In some embodiments, methods of making engineered cells can comprise stimulation, such as by contact with an anti-CD3 antibody or antigen-binding fragment thereof, or an anti-CD2 antibody immobilized on a surface, or by contact with a protein kinase C activator (e.g., bryostatin), optionally in conjunction with a calcium ionophore. For co-stimulation of an accessory molecule on the surface of the T cells, a ligand that binds the accessory molecule can be used. In some cases a population of T cells can be CD3-CD28 co-stimulated, for example, contacted with an anti-CD3 antibody and an anti-CD28 antibody, under conditions that can stimulate proliferation of the T cells.

In some examples, the engineered immune cell is an engineered NK cell that is derived from an isolated ESC or an induced stem cell (e.g., iPSC).

In some cases, engineered immune cells (e.g., engineered NK cells) disclosed herein can be derived from one or more isolated stem cells (e.g., isolated ESCs). In some cases, engineered immune cells disclosed herein can be derived from one or more induced stem cells (e.g., iPSCs).

Any one of the engineered cells disclosed herein can be or can be derived from an isolated stem cell (e.g., an ESC) or an induced stem cell (e.g., an iPSC). The isolated stem cell or the induced stem cell can be modified (e.g., genetically modified) at a genetic site disclosed herein to generate the engineered stem cell.

In some cases, pluripotency of stem cells (e.g., ESCs or iPSCs) can be determined, in part, by assessing pluripotency characteristics of the cells. Pluripotency characteristics can include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) ability to form teratomas comprising the three somatic lineages; and (vi) formation of embryoid bodies comprising cells from the three somatic lineages.

Any one of the engineered cells disclosed herein can be or can be derived from a hematopoietic stem cell. In some embodiments, the hematopoietic stem cell can be from a subject, for example, from bone marrow, or peripheral blood (e.g., a mobilized peripheral blood apheresis product, for example, mobilized by administration of GCSF, GM-CSF, mozobil, or a combination thereof).

In some cases, stem cells (e.g., ESCs or iPSCs) can be genetically modified to generate (e.g., induce differentiation into) CD34+ hematopoietic stem cells. The stem cells can be genetically modified to express any one of the transgenes (e.g., cytokines, receptors, etc.) as disclosed herein prior to, subsequent to, or during the induced hematopoietic stem cell differentiation. The stem cells can be genetically modified to reduce expression or activity of any one of the endogenous genes or polypeptides (e.g., cytokines, receptors, etc.) as disclosed herein prior to, subsequent to, or during the induced hematopoietic stem cell differentiation. In some cases, such a genetically modified CD34+ hematopoietic stem cell is or is a source of any one of the engineered cells of the present disclosure. One or more of the genetic modifications can be at a safe harbor genomic site disclosed herein.

In some examples, stem cells as disclosed herein can be cultured in APEL media with ROCKi (Y-27632) (e.g., at about 10 micromolar (μM)), SCF (e.g., at about 40 nanograms per milliner (ng/mL) of media), VEGF (e.g., at about 20 ng/mL of media), and BMP-4 (e.g., at about 20 ng/mL of media) to differentiate the stem cells into CD34+ hematopoietic stem cells.

In some cases, the CD34+ hematopoietic stem cells (e.g., genetically modified with one or more artificially-induced modifications of the present disclosure) can be induced to differentiate in to a committed immune cell, such as T cells or NK cells. As such, in some cases, the induced differentiation process generates any one of the engineered immune cells of the present disclosure.

In some examples, genetically modified CD34+ hematopoietic stem cells are cultured in the presence of IL-3 (e.g., about 5 ng/mL), IL-7 (e.g., about 20 ng/mL), IL-15 (e.g., about 10 ng/mL), SCF (e.g., about 20 ng/mL), and Flt3L (e.g., about 10 ng/mL) to differentiate into CD45+NK cells.

In some cases, the CD45+NK cells can be expanded in culture, e.g., in a media comprising IL-2, mbIL-21 aAPC using Gas Permeable Rapid Expansion (G-Rex) platform.

In some cases, iPSC-derived NK cells as disclosed herein can be cultured with one or more cytokines comprising IL-2, IL-15, or IL-21. In some cases, iPSC-derived NK cells as disclosed herein can be cultured with (e.g., for cell expansion) one or more cytokines selected from the group consisting of IL-2, IL-15, and IL-21. In some cases, iPSC-derived NK cells as disclosed herein can be cultured with two or more cytokines selected from the group consisting of IL-2, IL-15, and IL-21 (e.g., IL-2 and IL-15, IL-2 and IL-21, or IL-15 and IL-21), either simultaneously or sequentially in any order. In some cases, iPSC-derived NK cells as disclosed herein can be cultured with all of IL-2, IL-15, and IL-21, either simultaneous or sequentially in any order.

In some embodiments, engineered cells can be cultured in serum-free media.

Cells can be obtained from any suitable source for the generation of engineered cells. Cells can be primary cells. Cells can be recombinant cells. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. Cells can be derived from a healthy donor, from a patient diagnosed with cancer, or from a patient diagnosed with an infection. Cells can also be obtained from a cell therapy bank. Cells can also be obtained from whole blood, apheresis, or a tumor sample of a subject. A cell can be a tumor infiltrating lymphocytes (TIL). In some cases an apheresis can be a leukapheresis.

If the cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring or synthetic factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

A desirable cell population can also be selected prior to or after modification. A selection can include at least one of: magnetic separation, flow cytometric selection, and antibiotic selection.

In some embodiments, an engineered cell is used to manufacture a biologic, for example, an antibody or other protein-based therapeutic. In some embodiments, an engineered cell is a cell line, for example, a HEK cell.

### F. Vectors, Gene Editing Moieties, and Methods of Making Engineered Cells

A gene editing moiety can be used to introduce an artificially-induced modification in a genomic site of the disclosure.

The gene editing moiety as disclosed herein can comprise a CRISPR-associated polypeptide (Cas), zinc finger nuclease (ZFN), zinc finger associate gene regulation polypeptide, transcription activator-like effector nuclease (TALEN), transcription activator-like effector associated gene regulation polypeptides, meganuclease, natural master transcription factors, epigenetic modifying enzymes, recombinase, flippase, transposase, RNA-binding proteins (RBP), an Argonaute protein, any derivative thereof, any variant thereof, or any fragment thereof. In some embodiments, the gene editing moiety comprises a Cas protein, and the system further comprises a guide RNA (gRNA) which complexes with the Cas protein. In some embodiments, the gene editing moiety comprises an RBP complexed with a gRNA which is able to form a complex with a Cas protein. In some embodiments, the gRNA comprises a targeting segment which exhibits at least 80%, at least 85%, at least 90%, at least 91%, at least 92%, at least 93%, at least 94%, at least 95%, at least 96%, at least 97%, at least 98%, or at least 99%, or 100% sequence identity to a target polynucleotide. In some embodiments, the Cas protein substantially lacks DNA cleavage activity.

In some cases, a suitable gene editing moiety comprises CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof, any variant thereof; and any fragment thereof.

Non-limiting examples of Cas proteins include c2c1, C2c2, c2c3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Cas9 (Csn1 or Csx12), Cas1O, Cas1Od, Cas1O, Cas1Od, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cul966, and homologs or modified versions thereof.

In some cases, a dual nickase approach may be used to introduce a double stranded break. Cas proteins can be mutated at certain amino acids within either nuclease domains, thereby deleting activity of one nuclease domain and generating a nickase Cas protein capable of generating a single strand break. A nickase along with two distinct guide RNAs targeting opposite strands may be utilized to generate a DSB within a target site (often referred to as a “double nick” or “dual nickase” CRISPR system).

An insert sequence comprising a nucleotide sequence to be introduced to a genomic site (e.g., safe harbor locus of the disclosure) can also be introduced to the cell, together or separately from the gene editing moiety. The insert sequence can be flanked by one or more homology arms as disclosed herein to target integration into the genome, for example, by homology directed repair, homologous recombination, or any other suitable process for integration into the genome.

In some cases, the gene editing moiety as disclosed herein can be fused with an additional functional moiety (e.g., to form a fusion moiety), and non-limiting examples of a function of the additional functional moiety can include methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, and demyristoylation activity. For example, a fusion protein can be a fusion in a Cas protein and an effector or repressor functional moiety.

Alternatively or additionally, gene editing (e.g., knock in) or delivery of heterologous genetic material can utilize other viral and/or non-viral based gene transfer methods to introduce nucleic acids in host cells (e.g., stem cells, hematopoietic stem cells, immune cells, etc. as disclosed herein). In some embodiments, viral vectors can be used to introduce a gene editing moiety into a cell. Such methods can be used to administer an insert sequence of the present disclosure to cells in culture, or in a host organism. Viral vector delivery systems can include DNA and RNA viruses. Non-viral vector delivery systems can include DNA plasmids, RNA (e.g. a transcript of a vector described herein), naked nucleic acid, and nucleic acid complexed with a delivery vehicle, such as a liposome.

RNA or DNA viral based systems can be used to target specific cells and traffick the viral payload to the nucleus of the cell. Viral vectors can be used to treat cells in vitro or ex vivo, and the engineered cells can optionally be administered to a subject. Alternatively or additionally, viral vectors can be administered directly (in vivo) to the subject. Viral based systems can include retroviral, lentivirus, adenoviral, adeno-associated virus, and herpes simplex virus vectors for gene transfer. In some embodiments, integration in the host genome can occur with the retrovirus, lentivirus, and adeno-associated virus gene transfer methods, which can result in long term expression of the inserted transgene.

Methods of non-viral delivery of nucleic acids can include lipofection, nucleofection, microinjection, biolistics, virosomes, liposomes, immunoliposomes, polycation or lipid:nucleic acid conjugates, naked DNA, artificial virions, and agent-enhanced uptake of DNA. Cationic and neutral lipids that are suitable for efficient receptor-recognition lipofection of polynucleotides can be used.

Alternatively or additionally, antisense oligonucleotides can be utilized to suppress or silence expression of a target gene. Non-limiting examples of antisense oligonucleotides can include short hairpin RNA (shRNA), microRNA (miRNA), and small interfering RNA (siRNA).

Any suitable methods can be used to make engineered cells of the disclosure.

Methods of making engineered cells can comprise the use of a vector, for example, to introduce a nucleic acid sequence that comprises a transgene of the disclosure. A vector can be any genetic element, e.g., a plasmid, chromosome, virus, or transposon. Suitable vectors include, but are not limited to, plasmids, transposons, bacteriophages and cosmids. Vectors can contain polynucleotide sequences which are necessary to effect ligation or insertion of the insert sequence into a genomic site disclosed herein of a desired host cell and/or to effect the expression of the transgene. Such sequences can include promoter sequences to effect transcription, enhancer sequences to increase transcription, ribosomal binding site sequences and transcription and translation termination sequences. A vector can comprise a selectable marker gene.

A vector useful for the methods and compositions described herein can be a good manufacturing practices (GMP) compatible vector. For example, a GMP vector can be purer than a non-GMP vector.

### G. Methods of Use

An engineered cell of the disclosure can be used (e.g., administered) to treat a subject in need thereof. The subject can have or can be suspected of having a condition, such as a disease (e.g., cancer, tumor, tissue degeneration, fibrosis, etc.). A cell (e.g., a stem cell or a committed adult cell) can be obtained from the subject, and such cell can be cultured ex vivo and genetically modified to generate any subject engineered cell (e.g., any engineered NK cell) as disclosed herein. Subsequently, the engineered cell can be administered to the subject, for example, for adaptive immunotherapy.

The subject can be treated (e.g., administered with) a population of engineered cells (e.g., engineered NK cells) of the present disclosure for at least or up to about 1 dose, at least or up to about 2 doses, at least or up to about 3 doses, at least or up to about 4 doses, at least or up to about 5 doses, at least or up to about 6 doses, at least or up to about 7 doses, at least or up to about 8 doses, at least or up to about 9 doses, or at least or up to about 10 doses.

Engineered cells administered to a subject in need thereof can be autologous to the subject. Engineered cells administered to a subject in need thereof can be allogeneic to the subject, for example, fully HLA-matched, HLA matched at 1, 2, 3, 4, 5, 6, 7, or 8 HLA alleles, or at least 1, at least 2, at least 3, at least 4, at least 5, at least 6, at least 7, or at least 8 HLA alleles. Engineered cells administered to a subject in need thereof can be haploidentical to the subject. Engineered cells administered to a subject in need thereof can be from a donor that is related to the subject. Engineered cells administered to a subject in need thereof can be from a donor that is not related to the subject.

In certain embodiments, cryopreserved cells (e.g., engineered cells) are thawed and washed as described herein and allowed to rest for one hour at room temperature prior to activation using the methods of the present disclosure. In an aspect, a composition comprising an engineered cell can include a dosage form of a cell, e.g., a unit dosage form.

In one aspect, the present disclosure provides a method comprising (a) obtaining a cell from a subject; and (b) generating, from the cell, any one of the engineered cells (e.g., the engineered NK cell) disclosed herein. In some cases, the cell obtained from the subject is ESC.

In some cases, the cell (e.g., a fibroblast, such as an adult skin fibroblast) obtained from the subject is modified and transformed into an iPSC.

In one aspect, the present disclosure provides a method comprising administering to a subject in need thereof a population of NK cells comprising any one of the engineered cells (e.g., the engineered NK cell) disclosed herein. In some cases, the method can further comprise administering to the subject a co-therapeutic agent (e.g., a chemotherapeutic agent, anti-CD20 antibody, etc.).

In one aspect, the present disclosure provides a method comprising administering to a subject in need thereof any one of the compositions disclosed herein. In some cases, the composition can comprise (i) any one of the engineered cells (e.g., the engineered NK cell) disclosed herein and (ii) a co-therapeutic agent (e.g., a chemotherapeutic agent, anti-CD20 antibody, etc.).

Any one of the methods disclosed herein can be utilized to treat a target cell, a target tissue, a target condition, or a target disease of a subject.

In some embodiments, an engineered cell comprises a transgene encoding a chimeric polypeptide receptor at a genomic site of the disclosure, the chimeric polypeptide receptor recognizes an antigen expressed and/or presented by a target cell, triggering a desired response by the engineered cell upon recognition of the antigen.

A target disease can be a viral, bacterial, and/or parasitic infection; inflammatory and/or autoimmune disease; or neoplasm such as a cancer and/or tumor.

A target cell can be a diseased cell. A diseased cell can have altered metabolic, gene expression, and/or morphologic features. A diseased cell can be a cancer cell, a diabetic cell, or an apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

A variety of target cells can be killed using any one of the engineered cells (e.g., the engineered NK cell) disclosed herein. In some embodiments, an engineered cell comprises a transgene encoding a chimeric polypeptide receptor at a genomic site of the disclosure, and the chimeric polypeptide receptor recognizes an antigen expressed and/or presented by the target cell. A target cell can include a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem cells (iPSCs)) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can comprise a target nucleic acid. A target cell can be in a living organism. A target cell can be a genetically modified cell. A target cell can be a host cell.

A target cell can be a totipotent stem cell, however, in some embodiments of this disclosure, the term “cell” may be used but may not refer to a totipotent stem cell. A target cell can be a plant cell, but in some embodiments of this disclosure, the term “cell” may be used but may not refer to a plant cell. A target cell can be a pluripotent cell. For example, a target cell can be a hematopoietic cell that can differentiate into other cells in the hematopoietic cell lineage but may not be able to differentiate into any other non-hematopoietic cell. A target cell may be able to develop into a whole organism. A target cell may or may not be able to develop into a whole organism. A target cell may be a whole organism.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, or an apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring or synthetic factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

Non-limiting examples of cells which can be target cells include, but are not limited to, a lymphoid cell, such as a B cell, a T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell, Reticulocyte, Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver cells (e.g., Hepatocyte, or Kupffer cell), Cartilage/bone/muscle; bone cells, including Osteoblast, Osteocyte, Osteoclast, teeth cells, (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte, Epidermal basal cell, Keratinocyte of fingernails and toenails, Nail bed basal cell, Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell, Wet stratified barrier epithelial cells, Surface epithelial cell of stratified squamous epithelium of cornea, tongue, oral cavity, esophagus, anal canal, distal urethra and vagina, basal cell of epithelia of cornea, tongue, oral cavity, esophagus, anal canal, distal urethra and vagina, Urinary epithelium cell, Exocrine secretory epithelial cells, Salivary gland mucous cell, Salivary gland serous cell, Von Ebner's gland cell in tongue, Mammary gland cell, Lacrimal gland cell, Ceruminous gland cell in ear, Eccrine sweat gland dark cell, Eccrine sweat gland clear cell. Apocrine sweat gland cell, Gland of Moll cell in eyelid, Sebaceous gland cell, Bowman's gland cell in nose, Brunner's gland cell in duodenum, Seminal vesicle cell, Prostate gland cell, Bulbourethral gland cell, Bartholin's gland cell, Gland of Littre cell, Uterus endometrium cell, Isolated goblet cell of respiratory and digestive tracts, Stomach lining mucous cell, Gastric gland zymogenic cell, Gastric gland oxyntic cell, Pancreatic acinar cell, Paneth cell of small intestine, Type II pneumocyte of lung, Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell, Macula densa cell of kidney, Metabolism and storage cells, Barrier function cells (e.g., Lung, Gut, Exocrine Glands and Urogenital Tract), Kidney, Type I pneumocyte, Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte, Megakaryocyte, Monocyte, Connective tissue macrophage (various types), Epidermal Langerhans cell, Osteoclast, Dendritic cell, Microglial cell, Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, neurons, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell, Spermatozoon, Nurse cells, Ovarian follicle cell, Sertoli cell, Thymus epithelial cell, Interstitial cells, Interstitial kidney cells, and stem cells that are differentiated into or are to be differentiated into any cell type disclosed herein.

Of particular interest are cancer cells. In some embodiments, the target cell is a cancer cell. Non-limiting examples of cancer cells include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

In some cases, the target cell (e.g., B cells) as disclosed herein is associated or is suspected of being associated with an autoimmune disease. The subject being treated with any one of the engineered cell (e.g., engineered NK cell) of the present disclosure can have or can be suspected of having an autoimmune disease.

Non-limiting examples of an autoimmune disease can include acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, antibody-mediated transplantation rejection, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, hypergammaglobulinemia, idiopathic thrombocytopenic purpura (ITP), IgA nephropathy, IgG4-related sclerosing disease, immunoregulatory lipoproteins, inclusion body myositis, inflammatory bowel disease, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), lupus (SLE), lyme disease, Meniere's disease, microscopic polyangiitis, mixed connective tissue disease (MCTD), monoclonal gammopathy of undetermined significance (MGUS), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis (Granulomatosis with Polyangiitis (GPA)).

In some cases, the autoimmune disease comprises one or more members selected from the group comprising rheumatoid arthritis, type 1 diabetes, systemic lupus erythematosus (lupus or SLE), myasthenia gravis, multiple sclerosis, scleroderma, Addison's Disease, bullous pemphigoid, pemphigus vulgaris, Guillain-Barré syndrome, Sjogren syndrome, dermatomyositis, thrombotic thrombocytopenic purpura, hypergammaglobulinemia, monoclonal gammopathy of undetermined significance (MGUS), Waldenstrom's macroglobulinemia (WM), chronic inflammatory demyelinating polyradiculoneuropathy (CIDP), Hashimoto's Encephalopathy (HE), Hashimoto's Thyroiditis, Graves' Disease, Wegener's Granulomatosis, and antibody-mediated transplantation rejection (e.g., for tissue transplants such as renal transplant). In examples, the autoimmune disease can be type 1 diabetes, lupus, or rheumatoid arthritis.

In some cases the target disease is acute myeloid leukemia (AML). For example, any one of the engineered cells (e.g., the engineered NK cell) disclosed herein that comprises an artificially-induced modification at a genomic site can be administered to a subject in need thereof to treat AML. In some embodiments, the engineered cell is an engineered NK cell that comprises one or more of: (i) a chimeric polypeptide receptor comprising an antigen binding domain capable of binding to an antigen (e.g., CD33) as disclosed herein, (ii) a cytokine (e.g., IL-15) as disclosed herein, and (iii) a CD16 variant for enhanced CD16 signaling as disclosed herein. The engineered NK cell can be administered to a subject in need thereof to treat AML.

In some cases, the target disease is non-Hodgkin's lymphoma (NHL).

In some cases, the target disease is chronic lymphocytic leukemia (CLL).

In some cases, the target disease is B-cell leukemia (BCL). For example, any one of the engineered cells (e.g., the engineered NK cell) disclosed herein that comprises an artificially-induced modification at a genomic site can be administered to a subject in need thereof to treat BCL. In some embodiments, the engineered cell is an engineered NK cell that comprises one or more of: (i) a chimeric polypeptide receptor comprising an antigen binding domain capable of binding to CD19 as disclosed herein, (ii) a cytokine (e.g., IL-15) as disclosed herein, and (iii) a CD16 variant for enhanced CD16 signaling as disclosed herein. The engineered NK cell can be administered to a subject in need thereof to treat BCL.

In some cases, the target disease is non-small-cell lung carcinoma (NSCLC).

In some cases, the target cells form a tumor (e.g., a solid tumor). A tumor treated with the methods herein can result in stabilized tumor growth (e.g., one or more tumors do not increase more than 1%, more than 5%, more than 10%, more than 15%, or more than 20% in size, and/or do not metastasize). In some cases, a tumor is stabilized for at least about 1, at least about 2, at least about 3, at least about 4, at least about 5, at least about 6, at least about 7, at least about 8, at least about 9, at least about 10, at least about 11, or at least about 12, or more weeks. In some cases, a tumor is stabilized for at least about 1, at least about 2, at least about 3, at least about 4, at least about 5, at least about 6, at least about 7, at least about 8, at least about 9, at least about 10, at least about 11, or at least about 12, or more months. In some cases, a tumor is stabilized for at least about 1, at least about 2, at least about 3, at least about 4, at least about 5, at least about 6, at least about 7, at least about 8, at least about 9, or at least about 10, or more years. In some cases, the size of a tumor or the number of tumor cells is reduced by at least about 5%, at least about 10%, at least about 15%, at least about 20%, at least about 25, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more. In some cases, the tumor is completely eliminated, or reduced below a level of detection. In some cases, a subject remains tumor free (e.g. in remission) for at least about 1, at least about 2, at least about 3, at least about 4, at least about 5, at least about 6, at least about 7, at least about 8, at least about 9, at least about 10, at least about 11, at least about 12, or more weeks following treatment. In some cases, a subject remains tumor free for at least about 1, at least about 2, at least about 3, at least about 4, at least about 5, at least about 6, at least about 7, at least about 8, at least about 9, at least about 10, at least about 11, at least about 12, or more months following treatment. In some cases, a subject remains tumor free for at least about 1, at least about 2, at least about 3, at least about 4, at least about 5, at least about 6, at least about 7, at least about 8, at least about 9, at least about 10, or more years after treatment.

In some instances, engineered cells are formulated in a pharmaceutical composition comprising the engineered cells and a pharmaceutically-acceptable excipient, vehicle, carrier, or diluent. Pharmaceutical compositions can be formulated in a conventional manner using one or more physiologically acceptable carriers including excipients and auxiliaries which facilitate processing of the active compounds or cells into preparations which can be used pharmaceutically. Proper formulation can be dependent upon the route of administration chosen.

A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In certain embodiments, compositions can also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers can be included in an amount effective to maintain pH of the composition in an acceptable range.

In some embodiments, compositions can also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include but are not limited to sodium chloride, potassium chloride, sodium thiosulfate, sodium bisulfite and ammonium sulfate.

The pharmaceutical compositions described herein can be administered by any suitable administration route, including but not limited to, parenteral (e.g., intravenous, intratumoral, subcutaneous, intramuscular, intracerebral, intracerebroventricular, intra-articular, intraperitoneal, or intracranial), intranasal, buccal, sublingual, oral, or rectal administration routes. In some instances, the pharmaceutical composition is formulated for parenteral (e.g., intravenous, intratumoral, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous dispersions, liquids, gels, syrups, elixirs, slurries, suspensions and the like, for administration to a subject to be treated. In some embodiments, the pharmaceutical composition is formulated into solutions (for example, for IV administration). In some cases, the pharmaceutical composition is formulated as an infusion. In some cases, the pharmaceutical composition is formulated as an injection.

Parenteral administration can be, for example, by bolus injection or by gradual infusion or perfusion over time. Administration can also be by surgical deposition of a bolus or pellet of cells, or positioning of a medical device.

The pharmaceutical solid dosage forms described herein optionally include a compound or cell described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

### IV. EXAMPLES

### H. Example 1: Identification of Candidate Safe Harbor Loci

In a first approach, data were sourced from ENCODE, including data relating to DNaseI sensitivity, H3K4me3 histone methylation, H3K27ac histone acetylation, whole genome bisulfite sequencing, RNAseq, LaminB1, super enhancers, snoRNAs, lincRNAs, miRNAs, tRNAs, and ultraconserved elements. Data were for tissues and cell types including NK, CMP, T-cell, B-cell, CD14+ monocyte, liver, lung, muscle, and stomach. Files were processed to generate score matrices for sliding windows, which were quality checked, merged, and used to compute average signal, presence rate, and coefficient of variance. Data were processed to identify open chromatin regions based on DNase hypersensitivity and histone chip-seq. Open regions were then graded based on distance from the nearest coding gene, distance from the nearest cancer-associated gene, distance from snoRNAs, lincRNAs, and miRNAs, presence within or outside a gene transcription unit, presence within or outside an ultra-conserved region, and presence within or outside a vista enhancer region.

In a second approach, 300 candidate regions identified by single cell RNA-seq data were evaluated for similar criteria.

Candidates identified by both approaches were ranked and manually inspected for safe harbor suitability. Examples of identified candidate safe harbor sites identified in Genome Reference Consortium Human Build 38 (GRCh38/hg38) are shown in TABLE 1.

TABLE 1

| Safe | Safe | | Down- | |
|---|---|---|---|---|
| Harbor | Harbor | Upstream | stream | |
| Name | Type | Gene | Gene | Location |
| hSH1 | Sequential | FAU | ZNHIT2 | chr11: 65,117,969- |
| | promoter | | | 65,120,057 |
| hSH2 | Single | SYNGR1 | RPL3 | chr22: 39,319,072- |
| | promoter | | | 39,321,167 |
| hSH3 | Single | PIDD1 | RPLP2 | chr11: 808,403- |
| | promoter | | | 810,414 |
| hSH4 | Single | RNASEH1 | RPS7 | chr2: 3,574,031- |
| | promoter | | | 3,576,263 |
| hSH5 | Sequential | TMEM4 | S100A10 | chr1: 151,944,637- |
| | promoters | | | 151,946,598 |
| hSH6 | Sequential | ANAPC16 | DDIT4 | chr10: 72,259,705- |
| | promoters | | | 72,261,554 |
| hSH7 | Convergent | FOXB1 | ANXA2 | chr15: 60,126,969- |
| | promoters | | | 60,128,831 |
| hSH8 | Convergent | TEF | TOB2 | chr22: 41,413,106- |
| | promoters | | | 41,414,808 |
| hSH9 | Single | PHF14 | NDUFA4 | chr7: 10,940,150- |
| | promoter | | | 10,940,760 |
| hSH10 | Divergent | CEP95 | DDX5 | chr17: 64,506,290- |
| | promoter | | | 64,506,960 |
| hSH11 | Convergent | RPS4X | PIN4 | chrX: 72,268,950- |
| | promoter | | | 72,270,750 |
| hSH12 | Convergent | RPS16 | PLEKHG2 | chr19: 39,430,700- |
| | promoter | | | 39,431,400 |
| hSH13 | Convergent | RACK1 | TRIM41 | chr5: 181,235,790- |
| | promoter | | | 181,236,860 |
| hSH14 | Single | LYRM7 | HINT1 | chr5: 131,165,330- |
| | promoter | | | 131,165,510 |
| hSH15 | Divergent | MUS81 | CFL1 | chr11: 65,859,410- |
| | promoter | | | 65,860,050 |
| hSH16 | Convergent | COX6C | VPS13B | chr8: 99,877,580- |
| | promoter | | | 99,877,850 |

### I. Example 2: Reporter Gene Integration in Candidate Safe Harbor Loci

CRISPR/Cas9-mediated genome editing was utilized to integrate a reporter gene expression cassette at the candidate loci by homology directed repair. Donor plasmids were designed including homology arms to target integration at the candidate safe harbor locus, and GFP or RFP driven by a constitutive promoter (e.g., hEF-1a). The cassette also included a Woodchuck Hepatitis Virus (WHP) Posttranscriptional Regulatory Element (WPRE) to enhance reporter expression.

The donor plasmid and Cas9-gRNA ribonucleoproteins were co-transfected into cells, for example, H9 human embryonic stem cells (hESC) and ALD induced pluripotent stem cells (iPSC). Known safe harbor sites AAVS1 and H11 were used as controls.

Examples of gRNAs utilized are provided in TABLE 2.

TABLE 2

| Safe Harbor Name | gRNA |
|---|---|
| hSH1 | GATGAACCAGTCAAGTGATC |
| hSH2 | CGCGCTTGATTCTGAGGGTC |
| hSH3 | TTAGCGACTGCGGCCCTATC |
| hSH4 | GGCACCAAAGTACGAATCCT |
| hSH5 | TCAGGTAGTTCAGTGTAATC |
| hSH6 | CCCGAGAACTCACGTCAGAG |
| hSH7 | AGAGCGGGTTGGTCCTGTTT |
| hSH8 | GGGTTACGTAACGGGCTGCG |
| hAAVS1 | GATTCCCTTCTCAGGTTACG |
| hH11 | TGCACCTTCGCCCAAGTTAT |

Single cell clones were isolated by fluorescent activated cell sorting (FACS), and expanded. After the clones grew up, junction PCR was performed to confirm the integration of the reporter at the candidate safe harbor locus, and ddPCR was performed to screen for clones that only had one single copy of the reporter integrated.

### J. Example 3: Stability of Transgene Expression from Candidate Safe Harbor Loci in Human Embryonic Stem Cells (hESC)

Select clones generated as in Example 2 were evaluated for stability of transgene expression in human embryonic stem cells (hESC). hESC clones were maintained in culture for up to 8 passages, and the percentage of progeny that expressed the reporter was evaluated by flow cytometry.

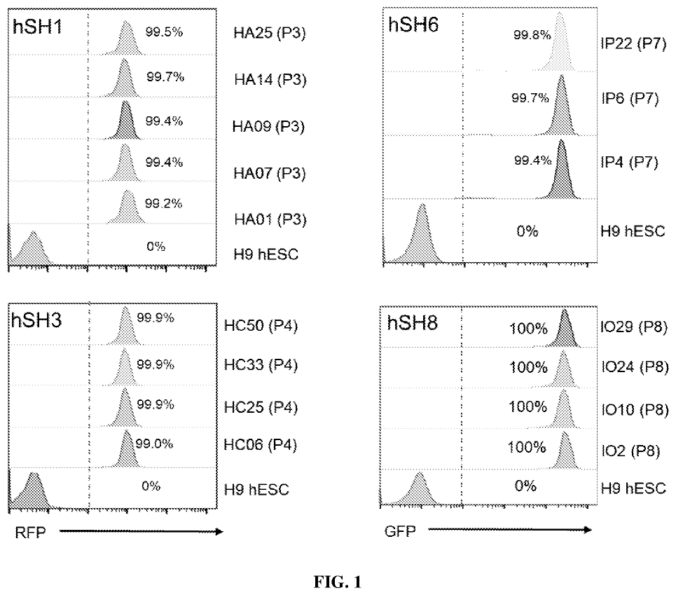

Clones with the expression cassette integrated at the hSH1 locus exhibited transgene expression in 99.2-99.7% of cells after three passages (FIG. 1, upper left panel).

Clones with the expression cassette integrated at the hSH3 locus exhibited transgene expression in 99-99.9% of cells after four passages (FIG. 1, lower left panel).

Clones with the expression cassette integrated at the hSH6 locus exhibited transgene expression in 99.4-99.8% of cells after seven passages (FIG. 1, upper right panel).

Clones with the expression cassette integrated at the hSH8 locus exhibited transgene expression in 100% of cells after eight passages (FIG. 1, lower right panel).

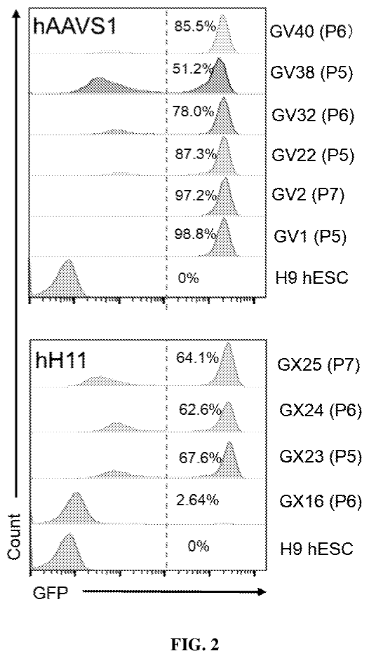

Clones with the expression cassette integrated at the hAAVS1 (control) locus exhibited transgene expression in 51.2-98.8% of cells after 5-7 passages (FIG. 2, upper panel). Notably, four of six clones exhibited considerable silencing of transgene expression by passage 5 or 6, with loss of expression in 12-48.8% of cells. Single cell PCR indicated that the GFP negative population originated from silencing rather than wild type cell contamination, as the junction PCR positivity rate was comparable between GFP negative and GFP positive populations.

Clones with the expression cassette integrated at the hH11 (control) locus exhibited transgene expression in 2.64-67.6% of cells after 5-7 passages (FIG. 2, lower panel). Notably, all clones exhibited considerable silencing of transgene expression, with loss of expression in 32.4-97.36% of cells. ddPCR on sorted cells indicated that the GFP negative population originated from silencing rather than wild type cell contamination, as the non-sorted, sorted GFP negative, and sorted GFP positive populations each has 1 GFP copy according to ddPCR.

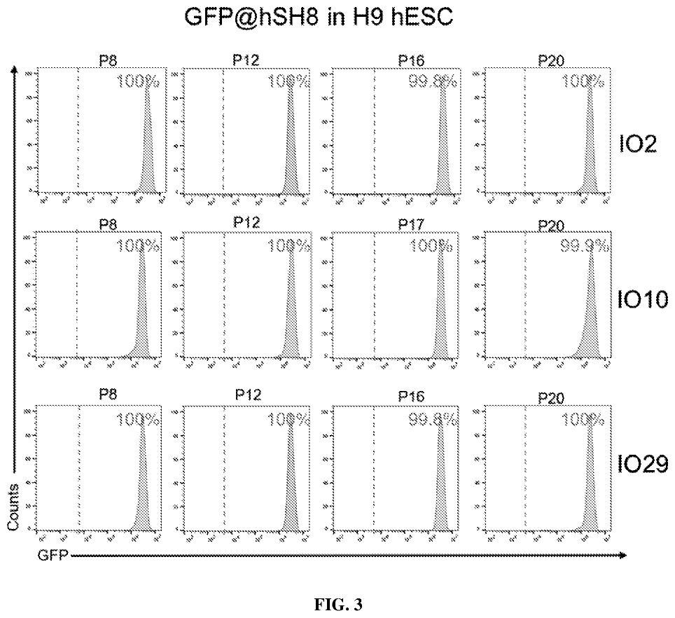

Clones with the expression cassette integrated at the hSH8 locus were further evaluated up to passage 20, and exhibited transgene expression in 99.9-100% of cells after 20 passages (FIG. 3, each row represents a different clone).

These data demonstrate that safe harbor loci of the present disclosure exhibit superior stability in transgene expression compared to existing safe harbor loci.

### K. Example 4: Stability of Transgene Expression from Candidate Safe Harbor Loci in ALD Induced Pluripotent Stem Cells (iPSC)

Select clones generated as in Example 2 were evaluated for stability of transgene expression in iPSC. iPSC clones were maintained in culture for up to 5 passages, and the percentage of progeny that expressed the reporter was evaluated by flow cytometry.

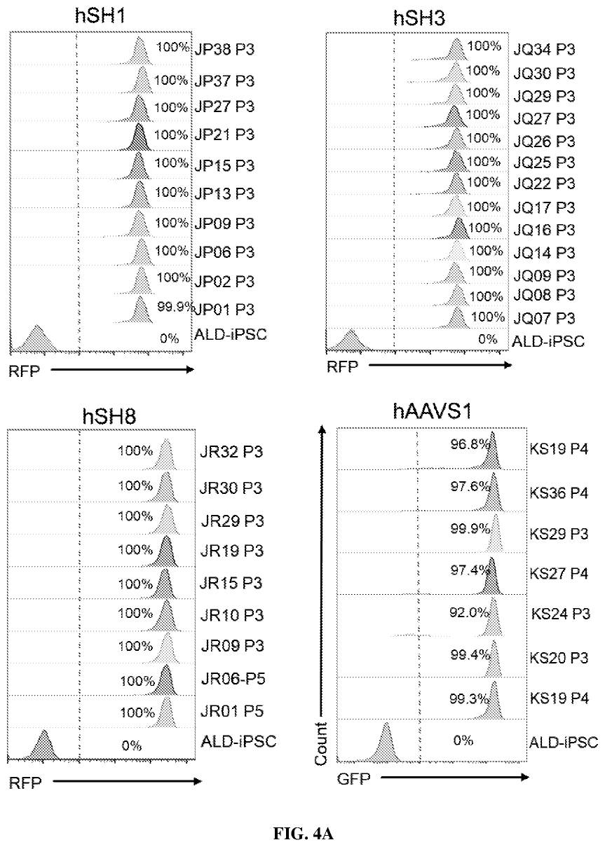

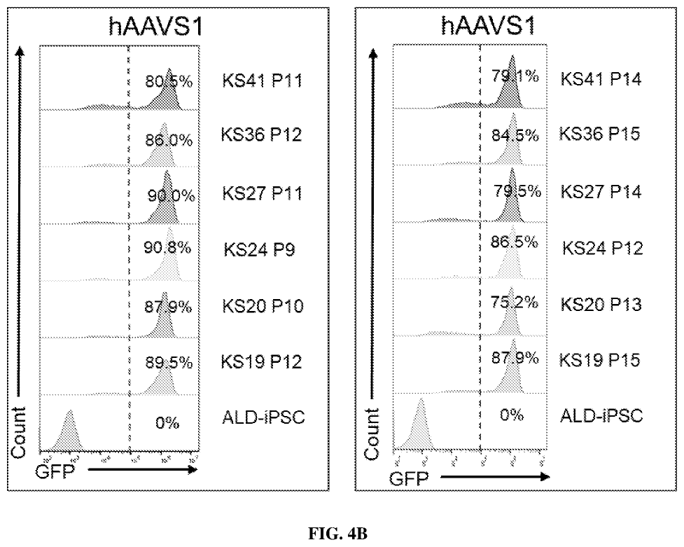

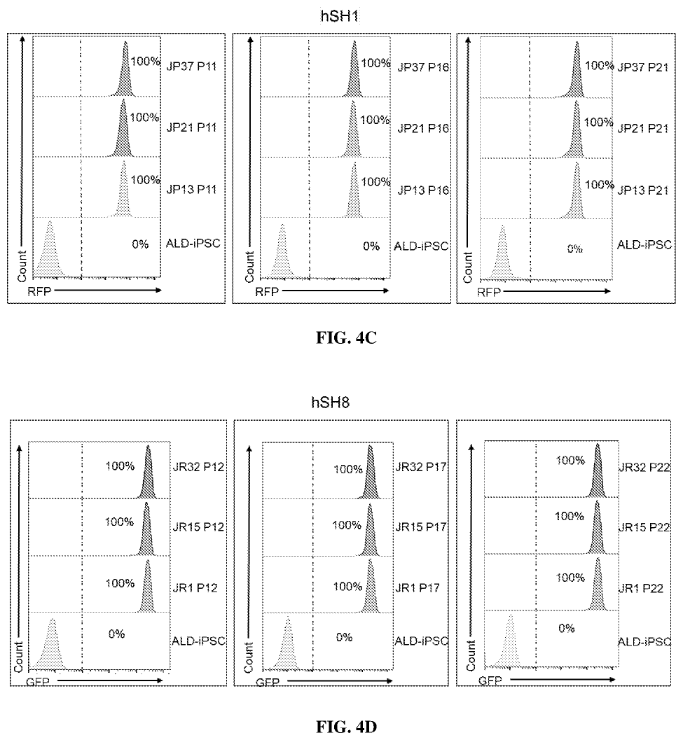

Clones with the expression cassette integrated at the hSH1 locus exhibited transgene expression in 99.9-100% of cells after three passages (FIG. 4A, upper left panel).

Clones with the expression cassette integrated at the hSH3 locus exhibited transgene expression in 100% of cells after three passages (FIG. 4A, upper right panel).

Clones with the expression cassette integrated at the hSH8 locus exhibited transgene expression in 100% of cells after three to five passages (FIG. 4A, lower left panel).

Clones with the expression cassette integrated at the hAAVS1 (control) locus exhibited transgene expression in 92-99.9% of cells after 3-4 passages (FIG. 4A, lower right panel). Notably, four of seven clones exhibited loss of expression in at least 2% of cells by passage 3-4.

At later passages, gradual loss of expression was observed in the clones with the expression cassette integrated at the hAAVS1 (control) locus (FIG. 4B). In contrast, for clones with integration of the expression cassette at hSH1 or hSH8, 100% of cells maintained high expression of the transgene to at least passage 21 or 22 (FIG. 4C and FIG. 4D, respectively).

These data demonstrate that safe harbor loci of the present disclosure exhibit superior stability in transgene expression compared to existing safe harbor loci.

### L. Example 5: Stability of Transgene Expression from Candidate Safe Harbor Loci in Stem Cells Following Differentiation into Embryoid Bodies (EB)

Select clones generated as in Example 2 were evaluated for stability of transgene expression following differentiation into embryoid bodies (EB). The percentage of progeny that expressed the reporter was evaluated by flow cytometry, and CD34 was used as a marker indicating differentiation.

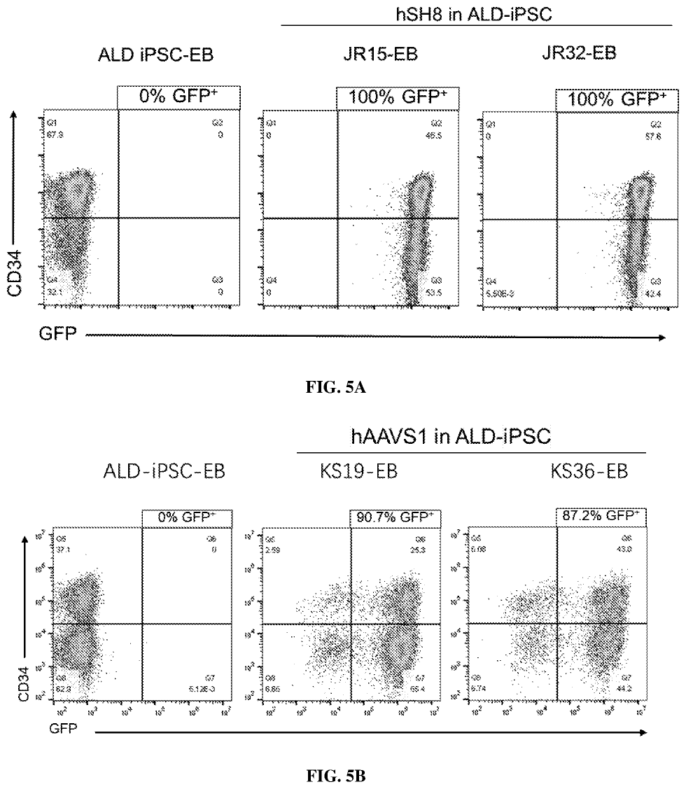

In a first experiment, hSH8 was evaluated in iPSC. CD34+ cells appeared on day 9 of the differentiation protocol, at which time 100% of live cells maintained transgene expression (FIG. 5A). In contrast, approximately 10-13% of cells with the expression cassette integrated at AAVS1 exhibited loss of transgene expression (FIG. 5B).

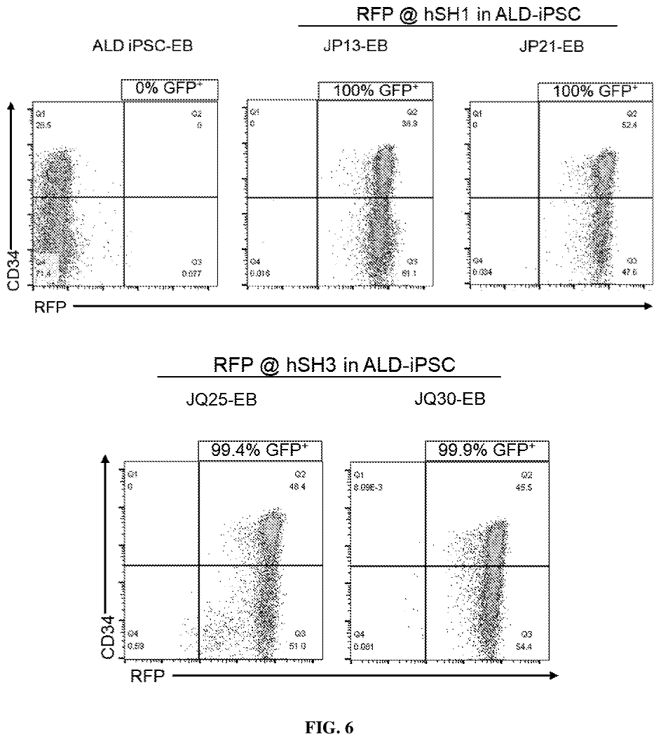

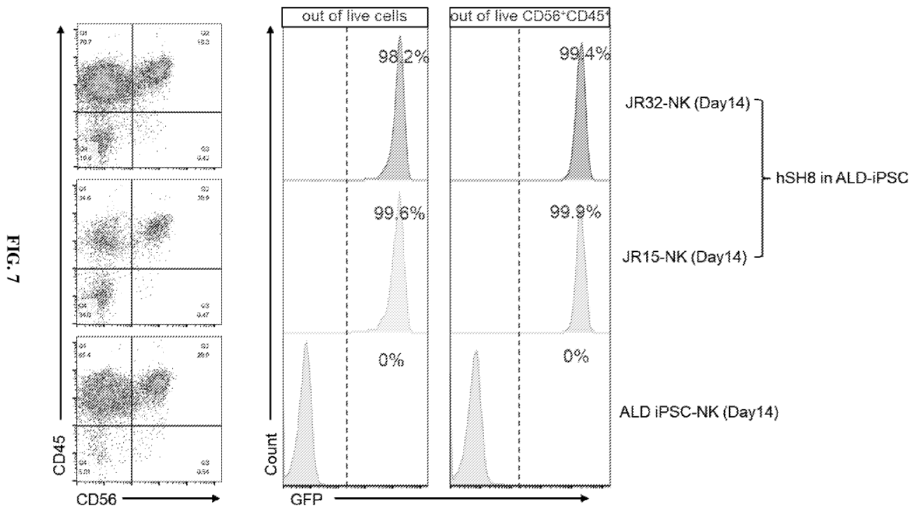

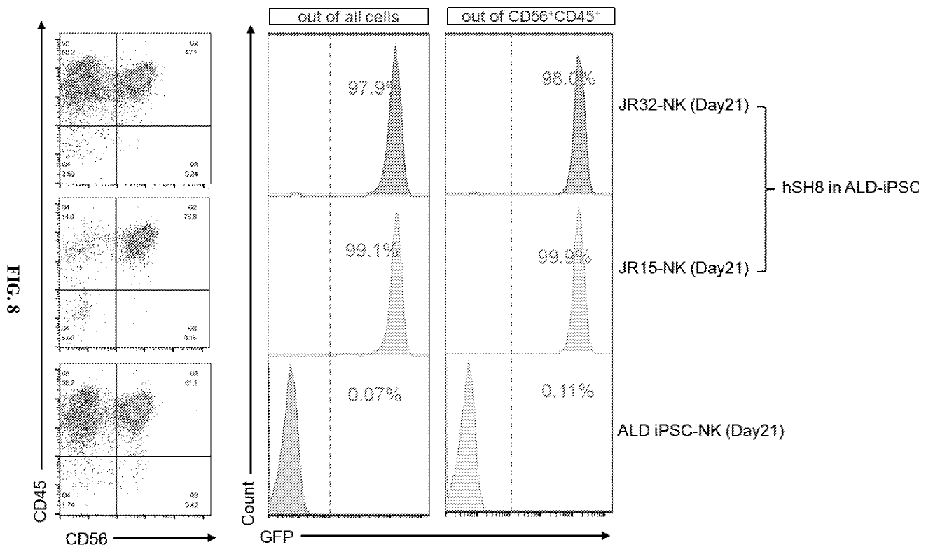

In a second experiment, hSH1 and hSH3 were evaluated in iPSC. CD34+ cells appeared on day 9 of the differentiation protocol, at which time 100% of live cells with the expression cassette integrated at hSH1 and 99.4-99.9% of live cells with the expression cassette integrated at hSH3 maintained transgene expression (FIG. 6).

These data demonstrate that that safe harbor loci of the present disclosure can facilitate stable transgene expression through the process of cell differentiation, including differentiation of stem cells into embryoid bodies.

### M. Example 6: Stability of Transgene Expression from Candidate Safe Harbor Loci in Stem Cells Following Differentiation into Natural Killer (NK) Cells

Select clones generated as in Example 2 were evaluated for stability of transgene expression following differentiation into NK cells. The percentage of progeny that expressed the reporter was evaluated by flow cytometry, and CD45 and CD56 were used as markers indicating NK cells.

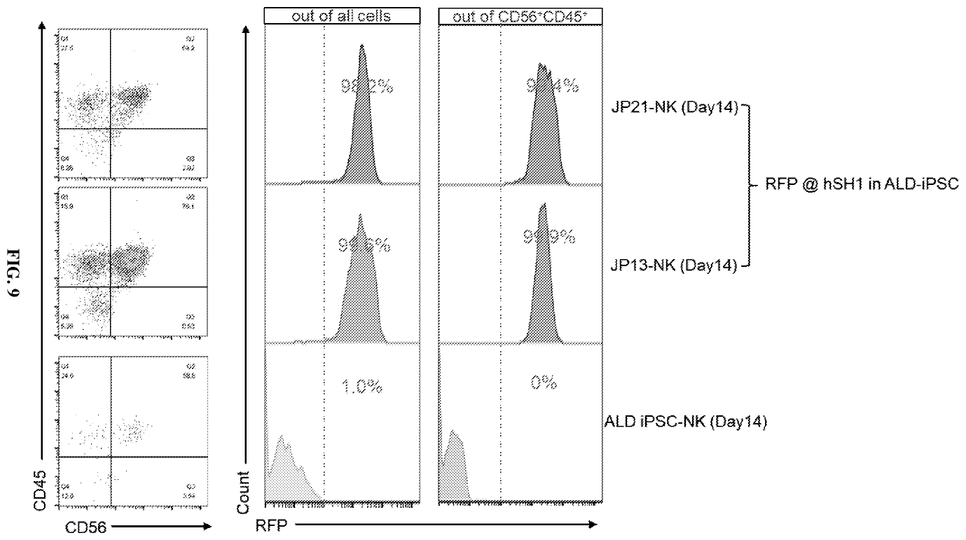

In a first experiment, hSH8 was evaluated in iPSC. CD45+CD56+NK cells appeared on day 14 of the differentiation protocol, at which time 98.2-99.6% of all live cells and 99.4-99.9% of live NK cells maintained transgene expression (FIG. 7). NK cells represented approximately 18-31% of live cells at this time (FIG. 7). By day 21, NK cells represented approximately 47-80% of all cells, and 97.9-99.1% of all cells maintained transgene expression, and 98-99.9% of NK cells maintained transgene expression (FIG. 8). Dead cells were not excluded by staining at this time point.

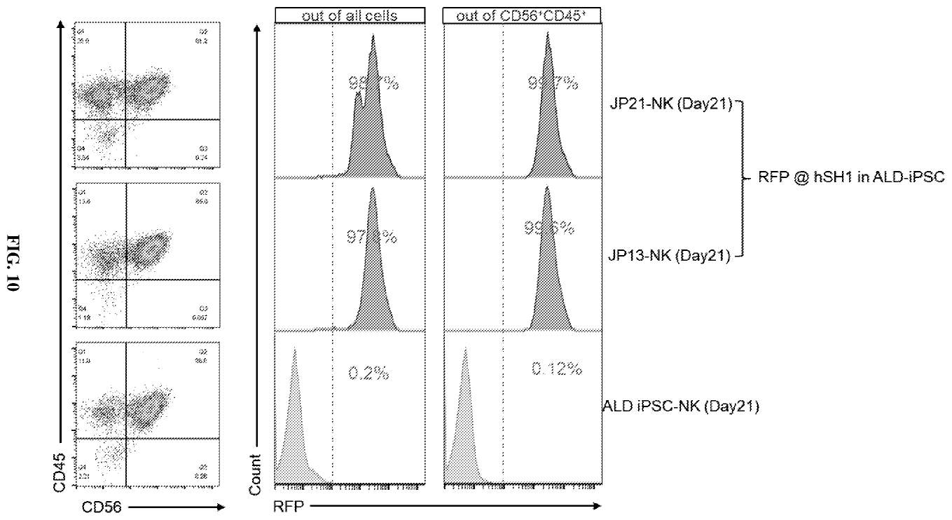

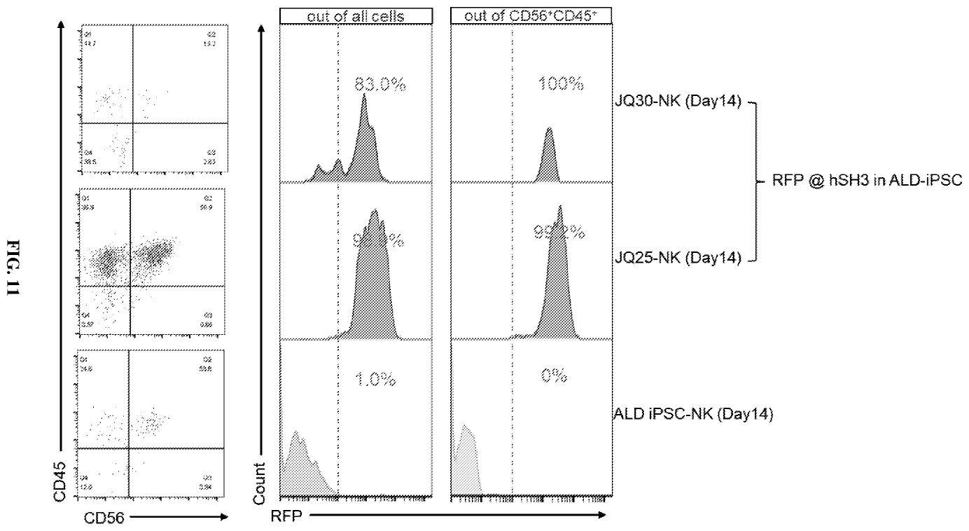

In a second experiment, hSH1 was evaluated in iPSC. CD45+CD56+NK cells appeared on day 14 of the differentiation protocol, at which time 98.2-99.6% of all cells and 99.4-99.9% of NK cells maintained transgene expression (FIG. 9). NK cells represented approximately 59-78% of cells at this time (FIG. 9). By day 21, NK cells represented approximately 61-87% of all cells, 97.8-98.7% of all cells maintained transgene expression, and 99.6-99.7% of NK cells maintained transgene expression (FIG. 10). Dead cells were not excluded by staining in this experiment.

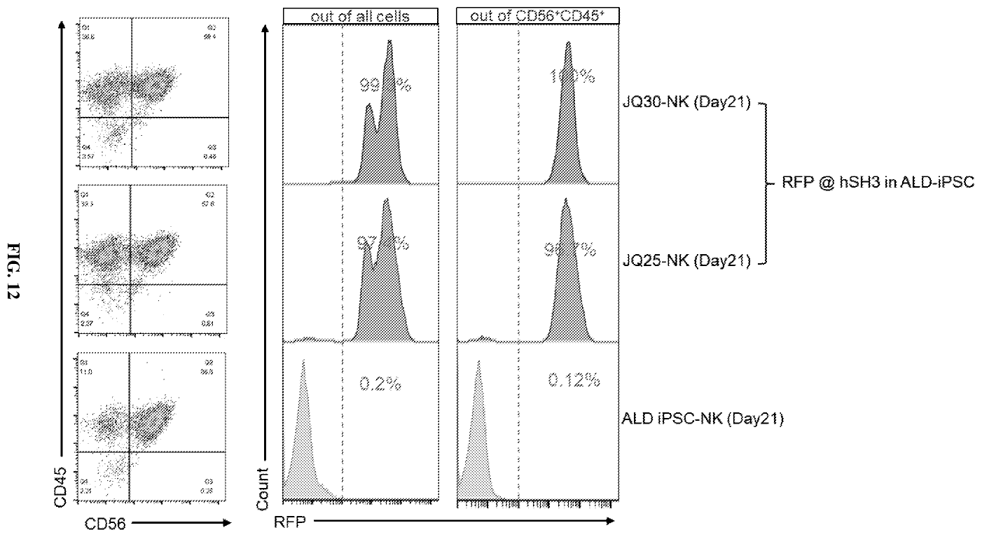

In a third experiment, hSH3 was evaluated in iPSC. CD45+CD56+NK cells appeared on day 14 of the differentiation protocol, at which time 83-98.9% of all cells and 99.2-100% of NK cells maintained transgene expression (FIG. 11). NK cells represented approximately 13-59% of live cells at this time (FIG. 11), however relatively few cells were available for evaluation for the clone that had 83% transgene expression. By day 21, NK cells represented approximately 59-87% of all cells, 97.4-99.3% of all cells maintained transgene expression, and 96.7-100% of NK cells maintained transgene expression (FIG. 12). Dead cells were not excluded by staining in this experiment.

These data demonstrate that that safe harbor loci of the present disclosure can facilitate stable transgene expression through the process of cell differentiation, including differentiation of stem cells into NK cells.

### N. Example 7: Stability of Transgene Expression from Candidate Safe Harbor Loci in Stem Cells Following Implant and Differentiation In Vivo

This example demonstrates stable transgene expression from candidate safe harbor loci of the disclosure in vivo.

hESC clones harboring a GFP expression cassettes at the hSH6 locus or hSH8 locus were generated as in Example 2. 5 million cells were injected into nude mice and after two months, spleen and teratoma tissues were harvested and processed for evaluation by flow cytometry and histopathology. An anti-human HLA antibody was used to identify cells originating from the injected hESC. Single cells were gated based on forward scatter area VS forward scatter height, and dead cells were gated out based on propidium iodide staining.

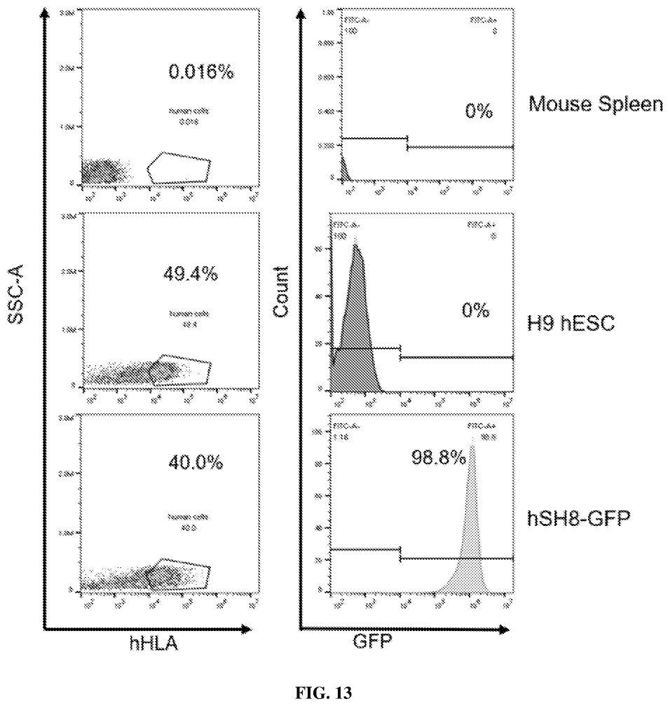

As shown in FIG. 13, approximately 40-50% of live single cells from the collected teratoma tissue originated from the implanted hESC clone based on hHLA staining. Of those cells, 98.8% of cells from an animal injected with a clone harboring the GFP expression cassette at hSH8 maintained GFP expression after implant and two months of differentiation into teratomas (FIG. 13).

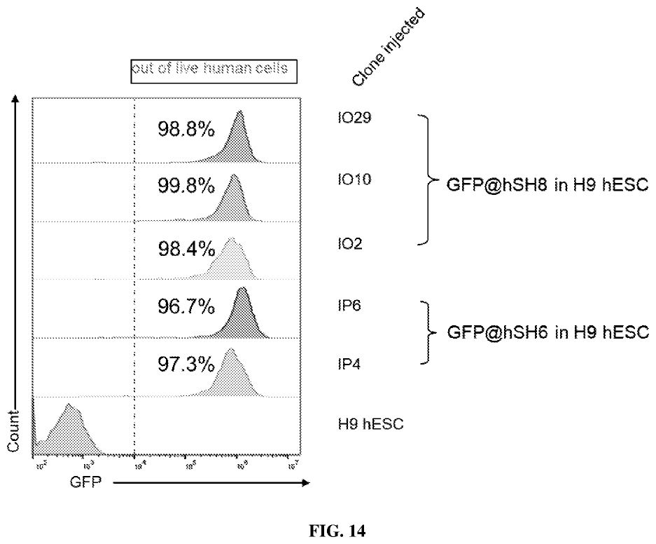

Additionally, 96.7-97.3% of human cells from animals injected with clones harboring the expression cassette at hSH6 maintained transgene expression, and 98.4-99.8% of human cells from animals injected with clones harboring the expression cassette at hSH8 maintained transgene expression (FIG. 14).

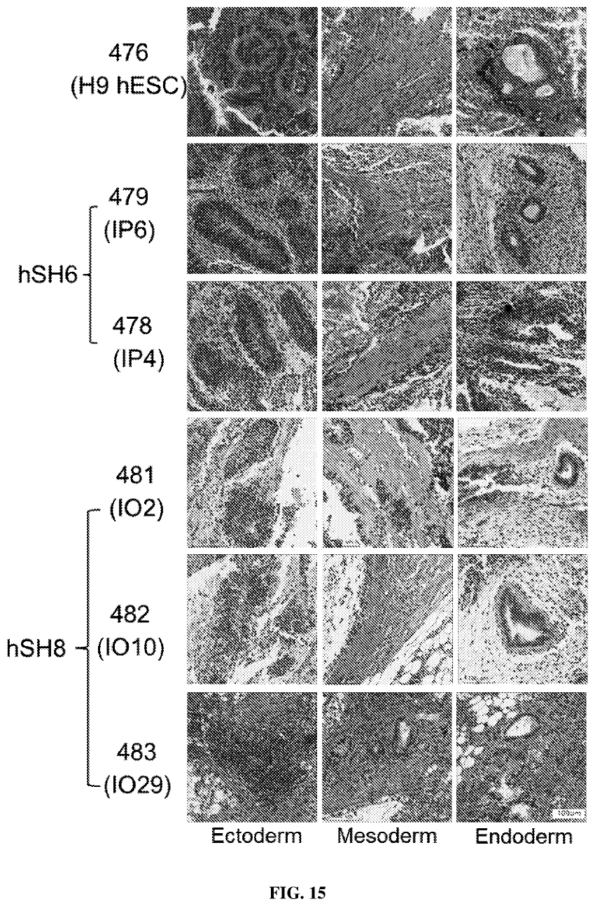

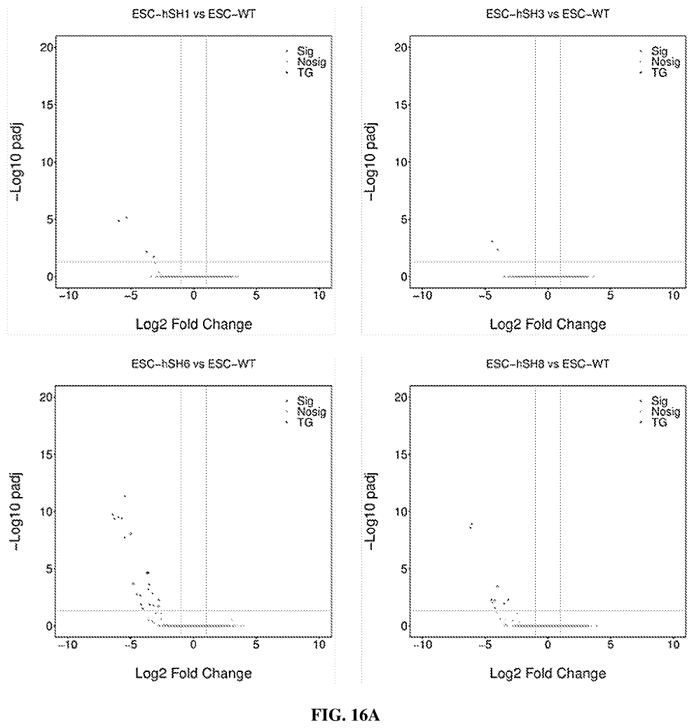

Sections of tissue processed for H&E staining demonstrated that clones with the expression cassette at hSH6 and hSH8 fully differentiated into ectoderm, mesoderm, and endoderm lineages (FIG. 15).

These data show that transgene expression from candidate safe harbor loci of the present disclosure is stable and sustained in vivo, including following a two month differentiation from hESC to teratomas.

### O. Example 8: Insertions in Safe Harbor Loci of the Disclosure have Minimal Impact on Local and Global Gene Expression

This example demonstrates that transgene insertion in safe harbor loci of the disclosure does not significantly disrupt expression of endogenous genes.

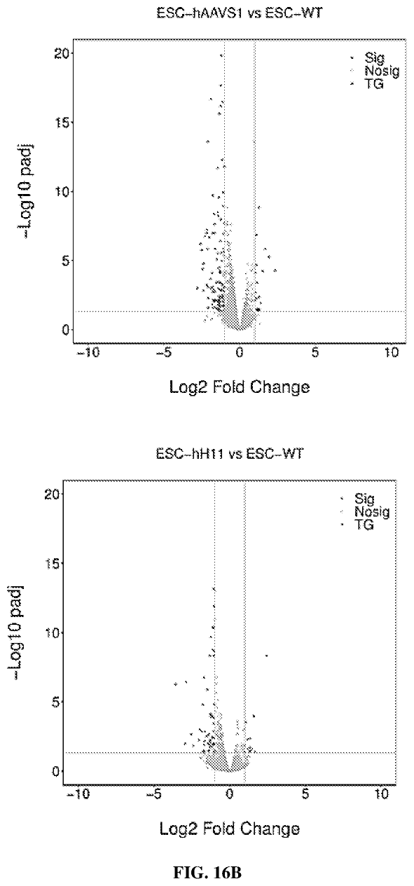

Select H9 hESC clones were generated as in Example 2 and maintained in culture for approximately 6-9 passages. RNA was extracted from the clones and processed for evaluation of gene expression by RNA seq. Clones with transgenes inserted in safe harbor loci of the disclosure (hSH1, hSH3, hSH6, and hSH8; FIG. 16A) exhibited very few differentially-expressed genes relative to control H9 hESC cultures maintained with transgene insertions in the AAVS1 or H11 loci (FIG. 16B).

These data demonstrate that transgene insertions in safe harbor loci of the present disclosure do not significantly disrupt expression of endogenous genes locally or globally.

### P. Example 9: Generation of Engineered Immune Cells

Safe harbor loci of the disclosure can be used as a site for insertion of an expression cassette for generation of engineered immune cells. For example, an expression cassette encoding a chimeric polypeptide receptor can be inserted in a safe harbor locus of the disclosure.

In an illustrative example, an expression cassette encoding a chimeric antigen receptor (CAR) is inserted into a safe harbor locus of the disclosure to generate stem cell clones, for example, any one of hSH1, hSH2, hSH3, hSH4, hSH5, hSH6, hSH7, or hSH8 in embryonic stem cells or induced pluripotent stem cells, utilizing genome editing technique of the disclosure, such as CRISPR/Cas9 genome editing as described in example 2.

The stem cells are differentiated into immune cells, for example, NK cells.

The engineered immune cells that express the chimeric polypeptide receptor are administered or are suitable for administration to a subject in need thereof to treat a disease, such as acute myeloid leukemia (AML), multiple myeloma (MM), Myelodysplastic syndrome (MDS), B cell leukemia, T cell leukemia, a solid tumor, or a blood cancer.

What is claimed:

1. A population of engineered cells, each engineered cell of the population comprising a transgene inserted in a genomic site, wherein upon insertion of the transgene into the genomic site, (i) more than 98.8% of the population maintains expression of the transgene for at least about 15 days, or (ii) more than 97.2% of the population maintains expression of the transgene for at least about 21 days; optionally, wherein the transgene is inserted in a genomic site that is not AAVS1, wherein upon insertion of the transgene into the genomic site that is not AAVS1, (iii) more than 68% of the population maintains expression of the transgene for at least about 15 days, or (iv) more than 65% of the population maintains expression of the transgene for at least about 21 days.

2. (canceled)

3. The population of engineered cells of claim 1, wherein the engineered cells are (i) pluripotent stem cells, or (ii) the lineage of said pluripotent stem cells:
e.g., wherein upon subjecting said pluripotent stem cells to differentiation towards a cell lineage, at least about 92% of the differentiating population maintains expression of the transgene, optionally wherein the population is subjected to the differentiation for at least about 14 to 21 days, optionally wherein the lineage cells comprise hematopoietic stem cells, NK cells, or T cells, or wherein the lineage cells are selected from the group consisting of embryoid bodies, mesoderm cells, endoderm cells, and ectoderm cells.

4. (canceled)

5. (canceled)

6. (canceled)

7. (canceled)

8. (canceled)

9. A population of engineered cells, each engineered cell of the population comprising an artificially-induced modification in a genomic site, wherein the artificially-induced modification effects no more than about 10-fold change in expression level of no more than about 100 endogenous genes.

10. (canceled)

11. The population of engineered cells of claim 9, wherein the nearest open reading frame to the genomic site in a 5′ or 3′ direction encodes a ribosomal protein, a ubiquitin family member, a ubiquitin modulator, a ubiquitin ligase, an apoptosis regulator, a cell cycle progression regulator, a transcription factor, a DNA damage response regulator, or a zinc finger-containing protein.

12. The population of engineered cells of claim 9, wherein the genomic site is an intergenic region between: (a) TOB2 and TEF; (b) FAU and ZNHIT2; (c) RPL3 and SYNGR1; (d) RPLP2 and PIDD1; (e) RPS7 and RNASEH1; (f) THEM4 and S100A10; (g) DDIT4 and ANAPC16; (h) ANXA2 and FOXB1; (i) NDUFA4 and PHF14; (j) DDX5 and CEP95; (k) PIN4 and RPS4X; (l) PLEKHG2 and RPS16; (m) TRIM41 and RACK1; (n) HINT1 and LYRM7; (o) CFL1 and MUS81; or (p) VPS13B and COX6C.

13. The population of engineered cells of claim 9, wherein the genomic site is adjacent to a promoter operatively coupled to one or more endogenous genes selected from the group consisting of FAU, ZNHIT2, RPL3, RPLP2, RPS7, TMEM4, S100A10, ANAPC16, DDIT4, FOXB1, ANXA2, TEF, TOB2, NDUFA4, DDX5, CEP95, PIN4, RPS4X, PLEKHG2, RPS16, TRIM41, RACK1, HINT1, CFL1, MUS81, VPS13B, and COX6C.

14. The population of engineered cells of claim 9, wherein the genomic site has at least 80% sequence identity to one or more sequences from the Genome Reference Consortium Human Build 38 (GRCh38/hg38) human genome selected from the group consisting of: (a) chr22: 41, 413, 106-41, 414, 808; (b) chr11: 65, 117, 969-65, 120, 057; (c) chr22: 39, 319, 072-39, 321, 167; (d) chr11: 808, 403-810, 414; (e) chr2: 3, 574, 031-3, 576, 263; (f) chr1: 151, 944, 637-151, 946, 598; (g) chr10: 72, 259, 705-72, 261, 554; (h) chr15: 60, 126, 969-60, 128, 831; (i) chr7: 10, 940, 150-10, 940, 760; (j) chr17: 64, 506, 290-64, 506, 960; (k) chrX: 72, 268, 950-72, 270, 750; (l) chr19: 39, 430, 700-39, 431, 400; (m) chr5: 181, 235, 790-181, 236, 860; (n) chr5: 131, 165, 330-131, 165, 510; (o) chr11: 65, 859, 410-65, 860, 050; and (p) chr8: 99, 877, 580-99, 877, 850.

15. (canceled)

16. The population of engineered cells of claim 9, wherein the genomic site is at least 1 kb from the nearest open reading frame, cancer-associated gene, or snoRNA-encoding, miRNA-encoding, or lincRNA-encoding gene in the genome.

17. (canceled)

18. (canceled)

19. (canceled)

20. (canceled)

21. (canceled)

22. The population of engineered cells of claim 1, wherein the transgene encodes an immune receptor, an antigen-recognition receptor, an NK receptor, a chimeric antigen receptor (CAR), a cytokine, a cytokine receptor, or a combination thereof, optionally wherein the chimeric antigen receptor further comprises a costimulatory domain.

23. (canceled)

24. (canceled)

25. (canceled)

26. (canceled)

27. (canceled)

28. (canceled)

29. (canceled)

30. The population of engineered cells of claim 9, wherein the engineered cells are stem cells, embryonic stem cells, induced pluripotent stem cells, immune cells, NK cells, T cells, or B cells.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The population of engineered cells of claim 9, wherein the genomic site is not AAVS1 or H11.

49. The population of engineered cells of claim 1, wherein the transgene is operably coupled to a constitutive promoter, an inducible promoter, or a tissue-specific promoter.

50. (canceled)

51. The population of engineered cells of claim 1, wherein the transgene is not operably coupled to an inducible promoter.

52. (canceled)

53. The population of engineered cells of claim 1, wherein (i) more than 98.8% of the population maintains constitutive expression of the transgene for at least about 15 days, or (ii) more than 97.2% of the population maintains constitutive expression of the transgene for at least about 21 days.

54. (canceled)

55. (canceled)

56. (canceled)

57. (canceled)

58. (canceled)

59. (canceled)

60. (canceled)

61. (canceled)

62. A vector configured for generation of the engineered cell of claim 1, the vector comprising a transgene and at least one homology arm, wherein the homology arm is at least 20 nucleotides in length and comprises a nucleotide sequence with at least 90% sequence identity to a corresponding sequence in an intergenic region between: (a) TOB2 and TEF; (b) FAU and ZNHIT2; (c) RPL3 and SYNGR1; (d) RPLP2 and PIDD1; (e) RPS7 and RNASEH1; (f) THEM4 and S100A10; (g) DDIT4 and ANAPC16; (h) ANXA2 and FOXB1; (i) NDUFA4 and PHF14; (j) DDX5 and CEP95; (k) PIN4 and RPS4X; (l) PLEKHG2 and RPS16; (m) TRIM41 and RACK1; (n) HINT1 and LYRM7; (o) CFL1 and MUS81; or (p) VPS13B and COX6C.

63. (canceled)

64. A method of making the engineered cell of claim 1, the method comprising introducing the transgene into the genomic site of a cell, optionally wherein the introduction of the transgene comprises providing a polynucleotide to be integrated into the genomic site by homology-directed repair, or wherein the introduction of the transgene comprises introducing a double-stranded break in the genomic site, optionally wherein the double-stranded break is introduced by a nuclease, optionally wherein the nuclease is a CRISPR-associated (Cas) nuclease, a transcription activator-like effector nuclease (TALEN), or a zinc finger nuclease.

65. (canceled)

66. (canceled)

67. (canceled)

68. (canceled)

69. The method of claim 64, wherein, 20 days subsequent to the introducing, (i) a percentage of cells expressing the transgene from a plurality of clones comprising the transgene inserted at the genomic site is higher than (ii) a percentage of cells expressing the transgene from a plurality of clones comprising the transgene inserted at an AAVS1 locus, or wherein (iii) an average expression level or duration of expression of the transgene from a plurality of clones comprising the transgene inserted at the genomic site is higher than (iv) an average expression level or duration of expression of the transgene from a plurality of clones comprising the transgene inserted at an AAVS1 locus.

70. (canceled)

71. (canceled)

72. The method of claim 69, wherein expression of the transgene inserted at the genomic site and expression of the transgene inserted at the AAVS1 locus are driven by the same or substantially the same promoter.

73. A pharmaceutical composition comprising the engineered cell of claim 1 and a pharmaceutically-acceptable excipient, carrier, vehicle, or diluent.

74. A method of treating a condition in a subject in need thereof, comprising administering to the subject the engineered cell of claim 1.
